(19) United States
(12) Patent Application Publication
(10) Pub. No.: US 2026/0015433 A1
(43) Pub. Date: Jan. 15, 2026

(54) METHOD OF PREPARING AN ANTIBODY WITH SITE-SPECIFIC MODIFICATIONS

(71) Applicant: SUZHOU BIOREINNO BIOTECHNOLOGY LIMITED COMPANY, Suzhou, Jiangsu (CN)

(72) Inventors: Lili WU, Suzhou, Jiangsu (CN); Ao JI, Suzhou, Jiangsu (CN); Wenxu HE, Suzhou, Jiangsu (CN)

(21) Appl. No.: 18/881,717

(22) PCT Filed: Aug. 22, 2023
(86) PCT No.: PCT/CN2023/114318

§ 371 Date: Jan. 6, 2025

(30) Foreign Application Priority Data
Aug. 22, 2022 (WO) PCT/CN2022/113992
Sep. 20, 2022 (WO) PCT/CN2022/119955
Nov. 11, 2022 (WO) PCT/CN2022/131519
Jan. 19, 2023 (WO) PCT/CN2023/073070

(51) Int. Cl. C07K 16/32; A61K 47/68; C07K 16/28; C07K 16/30

(52) U.S. Cl. CPC C07K 16/32; A61K 47/68031; A61K 47/6849; A61K 47/6851; C07K 16/2878; C07K 16/30; C07K 2317/24

(57) ABSTRACT

Provided is a method of preparing an antibody with site-specific modification. Three of four interchain disulfide bonds within the antibody are selectively reduced firstly. By the site-specific modification of the antibody, many kinds of ADCs are provided, such as ADC with D2, ADC with D1, ADC with D4, ADC with D6, ADC with D3, ADC with D0+D2, ADC with D0+D1, the bi-payload ADC with D1+D6, the bi-payload ADC with D1+D2, the bi-payload ADC with D1+D4, the bi-payload ADC with D2+D4, the bi-payload ADC with D6+D2, the bi-payload ADC with D6+D1, the bi-payload ADC with D3+D1, the bi-payload ADC with D3+D2, the bi-payload ADC with D1+D2, the bi-payload ADC with D1+D4 or the bi-payload ADC with D2+D4. As compared with conventional conjugation method, the homogeneity of antibody with site-specific modification from the method can be dramatically improved, which more than 55%, 65%, 70%, 80%, 85%, even to 90% or 95%. Meanwhile the method has simple manipulation and reduced cost without antibody engineering and enzymes engineering.

### CROSS-REFERENCE TO RELATED APPLICATIONS

This application claims the priority to PCT Application No. PCT/CN2022/113992, filed on Aug. 22, 2022, PCT Application No. PCT/CN2022/119955, filed on Sep. 20, 2022, PCT Application No. PCT/CN2022/131519, filed on Nov. 11, 2022, and PCT Application No. PCT/CN2023/073070, filed on Jan. 19, 2023. The contents of the prior PCT applications are considered as a part of the present disclosure and is incorporated herein in its entirety.

### TECHNICAL FIELD

The present application relates to a method of preparing an antibody with site-specific modifications. Specifically, the present application relates to a bio-conjugation process for preparing ADCs with improved homogeneity.

### BACKGROUND

The statements in this section merely provide background information related to the present disclosure and do not necessarily constitute prior art.

Antibody drug conjugates (ADCs) are antibody with modification in which a monoclonal antibody is linked to a small molecule drug with a stable linker. ADCs ideally combine the specificity of antibodies and high potency of cytotoxic drugs by delivering potent cytotoxic drugs to antigen-expressing cells, thereby enhancing their targeted cytotoxic activity. In contrast to traditional chemotherapeutic drugs, antibody drug conjugates target only antigen-expressing cancer cells so that healthy cells are less severely affected (Pettinato, Mark C. (2021) “Introduction to Antibody-Drug Conjugates.” Antibodies (Basel, Switzerland) 10(42): 42-52, Joubert N, Beck A, Dumontet C, Denevault-Sabourin C. (2020) “Antibody-Drug Conjugates: The Last Decade.” Pharmaceuticals (Basel). 13(9): 245-275.). ADCs have extensive potential therapeutic applications in several disease areas, especially in cancer, and become a novel targeted drug for disease treatment. Since the approvals of Mylotarg in 2000, so far fourteen ADC drugs have been approved by US Food and Drug Administration.

For drug attachment of ADCs, functional groups with high reactivity on both antibody and linker-payload (i.e., linker-drug) were used for the conjugation, to form stable covalent bonds. Conventional means of conjugation, i.e., covalent bonding of a drug moiety to an antibody via a linker, generally leads to a heterogeneous mixture of molecules where the drug moieties are attached at several sites on the antibody. For example, ADCs are usually produced by two conventional chemical strategies, lysine-based conjugation and cysteine from the reduction of interchain disulfide bond based conjugation. For cysteine from the reduction of interchain disulfide bond based conjugation, it comprises a step of reducing interchain disulfide bonds in the presence of various reductants, followed by nucleophilic reaction of thiol groups. In this conjugation process, ADCs are typically formed by conjugating one or more antibody cysteine thiol groups to one or more linker-payload moieties thereby generating a heterogeneous antibody drug conjugate mixture (for example, Adcetris) where the drug moieties are attached at several sites on the antibody. For an ADC with a Drug-Antibody-Ratio (DAR) around 4, the heterogeneous mixture typically contains a distribution of antibodies attached with drug moieties from 0 to about 8, or more. In addition, within each subgroup of conjugates with a particular integer ratio of drug moieties to a single antibody, there is a potentially heterogeneous mixture where the drug moiety is attached at various sites on the antibody. The heterogeneous mixture is so complex that each conjugation product potentially has different pharmacokinetic, toxicity and efficacy profiles. Meanwhile it is difficult and expensive to characterize and purify them. And the conventional non-specific conjugation and conjugate distribution are largely influenced by factors such as pH, temperature, concentration, salt concentration, and co-solvents, so establishing a robust conjugation process always is challenging.

A number of methods have been developed to improve the homogeneity of ADCs. For example, Genentech's THIOMAB technology is developed based on improve the homogeneity of ADCs through antibody engineering, by introducing cysteine in the primary sequence of the antibody and realizing site-directed coupling to improve the uniformity of the product (“Cysteine-Based Coupling: Challenges and Solutions”. Bioconjug Chem. 2021 Aug. 18; 32(8):1525-1534.).

US20210040145 discloses a 14-amino acid peptide Tub-tagf used to the C-terminus of any POI and catalyzes the addition of a variety of different tyrosine derivatives. Taking advantage of this enzyme, Tub-tag technology repurposed tubulin-tyrosine ligase for the attachment of functional moieties at the C-terminus of antibody to homogeneously generate antibody conjugates with DAR 2.

However, those technologies involve protein engineering and/or enzyme catalysis, so that those technologies suffer from several drawbacks, such as lower level of antibody expression, immunogenicity risk, complicated purification, and/or high cost. Meanwhile there is not effective chemical method to generate ADCs with high D6 selectivity.

Therefore, there is a need for developing a novel bio-conjugation process which can generate ADCs with high improved homogeneity.

### SUMMARY

The present application develops a method of preparing an antibody with site-specific modification. With the site-specific modification of an antibody, three of four interchain disulfide bonds with the antibody are selectively reduced firstly. By this way, the present application provides many kinds of ADCs with high homogeneity, such as the ADC with D2, the ADC with D1, the ADC with D4, the ADC with D6, the ADC with D3, The ADC with D1+D6, The ADC with D2+D4, The ADC with D1+D2, The ADC with D1+D4, the ADC with D0+D2, the ADC with D0+D1, the bi-payload ADC with D6+D2, the bi-payload ADC with D6+D1, the bi-payload ADC with D3+D2, the bi-payload ADC with D3+D1, the bi-payload ADC with D1+D2, the bi-payload ADC with D1+D4 or the bi-payload ADC with D2+D4. As compared with conventional conjugation process, the homogeneity of ADCs is up to 55%, 65%, 70%, 80%, 85%, even to 90% or 95%. Further, when increasing the molar ratio of TCEP and the antibody, the method is with less reduction time cost. Meanwhile the method has simple manipulation and reduced cost without antibody engineering and enzymes engineering. The ADCs with improved homogeneity generated by the method of the present application further have optimized safety and efficacy.

On the one aspect, the present application provides a method of preparing antibody with site-specific modification, which characterized in that, the site-specific modification is that three interchain disulfide bonds within the antibody are reduced selectively, the method comprises that using tris (2-carboxyethyl) phosphine (TCEP) or salt thereof and transition metal ions together.

On the second aspect, the present application provides a method of preparing an antibody with site-specific modification, which characterized in that, the site-specific modification is that three interchain disulfide bonds within the antibody are reduced selectively, two of the three interchain disulfide bonds are in the Fab region and one is in the hinge region of the antibody, the method comprises that using TCEP or salt thereof and transition metal ions together.

On the third aspect, the present application provides a method of preparing an antibody with site-specific modification, which characterized in that, the site-specific modification is that three interchain disulfide bonds within the antibody are reduced selectively, two of the three interchain disulfide bonds are in the Fab region and one is in the hinge region of the antibody, the method comprises the following steps:
(a) incubating TCEP or salt thereof and transition metal ions in the presence of an antibody in a buffer system to selectively reduce interchain disulfide bonds within the antibody to afford the antibody bearing reduced thiol groups, the molar ratio of TCEP and the antibody is 3:1-15:1, optionally, the molar ratio of TCEP and the antibody is 3:1-6:1.

On the fourth aspect, the present application provides a method of preparing an antibody with site-specific modifications, which characterized in that, the method comprises the method of the present application, and also comprises the following steps:
(B1) introducing oxidant to selectively re-oxidize the reduced thiol groups resulted from step (a), optionally, re-oxidize the reduced thiol groups in Fab region, preferably, removing the excessive oxidant to purify the oxidized products;
(C1) introducing the metal chelators and modification reagent 1 to react with the remained thiol groups resulted from step (B1), wherein, the modification reagent 1 is an end capping reagent, a first linker-payload or a first thiobridge reagent, optionally, the first thiobridge reagent bears the first linker-payload or reactive groups.

On the fifth aspect, the present application provides a method of preparing an antibody with site-specific modification, which characterized in that, the method comprises the method of the present application, and also comprises the following steps:
(b) introducing the metal chelators and the modification reagent 1 to react with the reduced thiol groups resulted from step (a).

On the sixth aspect, the present application provides an antibody with site-specific modification prepared by the method of the present application.

On the seventh aspect, the present application provides a pharmaceutical composition comprising the antibody with site-specific modification according to the present application and one or more of pharmaceutically acceptable carrier.

On the eighth aspect, the present application provides use of TCEP or salt thereof in the preparation of the antibody with site-specific modification according to the present application.

On the ninth aspect, the present application provides use of the antibody with site-specific modification according to the present application in the manufacture of a therapeutic agent for diagnosing, preventing or treating a disease.

On the tenth aspect, the present application provides a method of preventing or treating a disease in a subject in need thereof, comprising administrating to the subject a therapeutically effective amount of the antibody with site-specific modification according to the present application.

### BRIEF DESCRIPTION OF THE DRAWINGS

FIG. 1 shows HIC-HPLC (Hydrophobic interaction chromatography-High performance liquid chromatography) of Trastuzumab-[MC-VC-PAB-MMAE]₆ conjugate of example 1.

FIG. 2 shows HIC-HPLC of Sacituzumab-[MC-VC-PAB-MMAE]₆ conjugate of example 2.

FIG. 3 shows HIC-HPLC of Belantamab-[MC-VC-PAB-MMAE]₆ conjugate of example 3.

FIG. 4 A-D show HIC-HPLC of Trastuzumab-[MC-VC-PAB-MMAE]₆ conjugate of example 4-7 (the molar ratio of TCEP and the antibody is 3:1, 3.2:1, 5:1, 6:1).

FIG. 5 A-F show HIC-HPLC of Trastuzumab-[MC-VC-PAB-MMAE]₆ conjugate of example 8-13 (the molar ratio of TCEP and the antibody is 8:1, 9:1, 10:1, 11:1, 12:1, 13:1).

FIG. 6 shows HIC-HPLC of Trastuzumab-[MC-VC-PAB-MMAE]₆ conjugate of example 14 (the molar ratio of Zn²⁺ and TCEP is 0.25:1).

FIG. 7 shows HIC-HPLC of Trastuzumab-[MC-VC-PAB-MMAE]₆ conjugate of example 15 (the molar ratio of Zn²⁺ and TCEP is 0.5:1).

FIG. 8 shows HIC-HPLC of Trastuzumab-[MC-VC-PAB-MMAE]6 conjugate of example 16 (the molar ratio of Zn²⁺ and TCEP is 1:1).

FIG. 9 shows HIC-HPLC of Trastuzumab-[MC-VC-PAB-MMAE]6 conjugate of example 17 (the molar ratio of Zn²⁺ and TCEP is 2:1).

FIG. 10 shows HIC-HPLC of Trastuzumab-[MC-VC-PAB-MMAE]6 conjugate of example 18 (the molar ratio of Zn²⁺ and TCEP is 3:1).

FIG. 11 shows HIC-HPLC of Trastuzumab-[MC-VC-PAB-MMAE]₆ conjugate of example 19 (the molar ratio of Zn²⁺ and TCEP is 4:1).

FIG. 12 shows HIC-HPLC of Trastuzumab-[MC-VC-PAB-MMAE]₆ conjugate of example 20 (the molar ratio of Zn²⁺ and TCEP is 7.5:1).

FIG. 13 A-D show HIC-HPLC of Trastuzumab-[MC-VC-PAB-MMAE]₆ conjugate of examples 21-24 (the molar ratio of Zn²⁺ and TCEP is 12:1, 27.27:1, 0.11:1, 0.22:1).

FIG. 14 A-D show HIC-HPLC of Trastuzumab-[MC-VC-PAB-MMAE]₆ conjugate of examples 25-28 (the molar ratio of Zn² and TCEP is 0.44:1, 0.66:1, 0.88:1, 1.67:1).

FIG. 15 A-D show HIC-HPLC of Trastuzumab-[MC-VC-PAB-MMAE]₆ conjugate of comparative examples 1-4 (the molar ratio of Zn²⁺ and TCEP is 0).

FIG. 16 A-D show HIC-HPLC of Trastuzumab-[MC-VC-PAB-MMAE]₆ conjugate of examples 29-32 (the incubation time in step (1) is different).

FIG. 17 A-D show HIC-HPLC of Trastuzumab-[MC-VC-PAB-MMAE]₆ conjugate of examples 33-36 (the incubation temperature in step (1) is different).

FIG. 18 shows HIC-HPLC of Trastuzumab-[MC-VC-PAB-MMAE]₆ conjugate prepared by using the Bis-Tris buffer (the pH value is 6.7) of example 37.

FIG. 19 shows HIC-HPLC of Trastuzumab-[MC-VC-PAB-MMAE]₆ conjugate prepared by using the PIPES buffer (the pH value is 6.7) of example 38.

FIG. 20 shows HIC-HPLC of Trastuzumab-[MC-VC-PAB-MMAE]₆ conjugate prepared by using the MOPS buffer (the pH value is 6.7) of example 39.

FIG. 21 shows HIC-HPLC of Trastuzumab-[MC-VC-PAB-MMAE]₆ conjugate prepared by using the BES buffer (the pH value is 6.7) of example 40.

FIG. 22 shows HIC-HPLC of Trastuzumab-[MC-VC-PAB-MMAE]6 conjugate prepared by using the HEPES buffer (the pH value is 6.7) of example 41.

FIG. 23 shows HIC-HPLC of Trastuzumab-[MC-VC-PAB-MMAE]6 conjugate prepared by using the DIPSO buffer (the pH value is 7.4) of example 42.

FIG. 24 shows MOBS of Trastuzumab-[MC-VC-PAB-MMAE]6 conjugate prepared by using the MOBS buffer (the pH value is 7.4) of example 43.

FIG. 25 shows HIC-HPLC of Trastuzumab-[MC-VC-PAB-MMAE]₆ conjugate prepared by using the MOPSO buffer (the pH value is 7.4) of example 44.

FIG. 26 shows HIC-HPLC of Trastuzumab-[MC-VC-PAB-MMAE]₆ conjugate prepared by using the TES buffer (the pH value is 7.4) of example 45.

FIG. 27 shows HIC-HPLC of Trastuzumab-[MC-VC-PAB-MMAE]₆ conjugate prepared by using the ACES buffer (the pH value is 7.4) of example 46.

FIG. 28 shows HIC-HPLC of Trastuzumab-[MC-VC-PAB-MMAE]₆ conjugate prepared by using the TAPSO buffer (the pH value is 7.4) of example 47.

FIG. 29 shows HIC-HPLC of Trastuzumab-[MC-VC-PAB-MMAE]₆ conjugate prepared by using the PB (the pH value is 6.7) of comparative example 5.

FIG. 30 shows HIC-HPLC of Trastuzumab-[MC-VC-PAB-MMAE]₆ conjugate prepared by using the ADA buffer (the pH value is 6.7) of comparative example 6.

FIG. 31 shows HIC-HPLC of Trastuzumab-[MC-VC-PAB-MMAE]₆ conjugate prepared by using the BES buffer (the pH value is 6.4) of example 48.

FIG. 32 shows HIC-HPLC of Trastuzumab-[MC-VC-PAB-MMAE]₆ conjugate prepared by using the BES buffer (the pH value is 6.7) of example 49.

FIG. 33 shows HIC-HPLC of Trastuzumab-[MC-VC-PAB-MMAE]₆ conjugate prepared by using the BES buffer (the pH value is 7.0) of example 50.

FIG. 34 shows HIC-HPLC of Trastuzumab-[MC-VC-PAB-MMAE]₆ conjugate prepared by using the BES buffer (the pH value is 6.4) of example 51.

FIG. 35 shows HIC-HPLC of Trastuzumab-[MC-VC-PAB-MMAE]₆ conjugate prepared by using 20 mM BES buffer of example 52.

FIG. 36 shows HIC-HPLC of Trastuzumab-[MC-VC-PAB-MMAE]6 conjugate prepared by using 40 mM BES buffer of example 53.

FIG. 37 shows HIC-HPLC of Trastuzumab-[MC-VC-PAB-MMAE]6 conjugate prepared by using 60 mM BES buffer of example 54.

FIG. 38 shows HIC-HPLC of Trastuzumab-[MC-VC-PAB-MMAE]6 conjugate prepared by using 80 mM BES buffer of example 55.

FIG. 39 shows HIC-HPLC of Trastuzumab-[Bismaleimide-DBCO]₃ conjugate of example 56.

FIG. 40 A shows HIC-HPLC of Trastuzumab-[MC-GGFG-DXd]₆ conjugate of example 57; B shows HIC-HPLC of Trastuzumab-[MC-GGFG-DXd]₆[MC-VC-PAB-MMAE]₂ conjugate of example 57.

FIG. 41 A shows HIC-HPLC of Trastuzumab-[MC-VC-PAB-MMAE]₆ conjugate of example 58; B shows HIC-HPLC of Trastuzumab-[MC-VC-PAB-MMAE]₆[Maleimide-PEG4-N3-DBCO-Cy3]₁ of example 58.

FIG. 42 A shows HIC-HPLC of Trastuzumab-[Maleimide]₆ conjugate of example 59; B shows HIC-HPLC of Trastuzumab-[Maleimide]₆[MC-VC-PAB-MMAE]₂ conjugate of example 59.

FIG. 43 shows HIC-HPLC of Trastuzumab-[Maleimide]₆[Maleimide-PEG4-N3-DBCO-Cy3]₁ conjugate of example 60.

FIG. 44 shows HIC-HPLC of Trastuzumab-[MC-VC-PAB-MMAE]₂ conjugate of example 61.

FIG. 45 shows HIC-HPLC of Trastuzumab-[MC-VC-PAB-MMAE]₂ conjugate of example 62.

FIG. 46 shows HIC-HPLC of Trastuzumab-[MC-VC-PAB-MMAE]₂ conjugate of example 63.

FIG. 47 shows HIC-HPLC of Trastuzumab-[MC-VC-PAB-MMAE]₂ conjugate of example 64.

FIG. 48 shows HIC-HPLC of Trastuzumab-[MC-VC-PAB-MMAE]₂ conjugate of example 65.

FIG. 49 shows HIC-HPLC of Trastuzumab-[MC-VC-PAB-MMAE]₂ conjugate of example 66.

FIG. 50 shows HIC-HPLC of Trastuzumab-[MC-VC-PAB-MMAE]₂ conjugate of example 67.

FIG. 51 shows HIC-HPLC of Trastuzumab-[MC-VC-PAB-MMAE]₂ conjugate of example 68.

FIG. 52 shows HIC-HPLC of Trastuzumab-[MC-VC-PAB-MMAE]₂ conjugate of example 69.

FIG. 53 shows HIC-HPLC of Trastuzumab-[MC-VC-PAB-MMAE]₂ conjugate of example 70.

FIG. 54 shows HIC-HPLC of Trastuzumab-[MC-VC-PAB-MMAE]₂ conjugate of example 71.

FIG. 55 shows HIC-HPLC of Trastuzumab-[MC-VC-PAB-MMAE]₂ conjugate of example 72.

FIG. 56 shows HIC-HPLC of Trastuzumab-[MC-VC-PAB-MMAE]₂ conjugate of example 73.

FIG. 57 shows HIC-HPLC of Trastuzumab-[MC-VC-PAB-MMAE]₂ conjugate of example 74.

FIG. 58 shows HIC-HPLC of Trastuzumab-[MC-VC-PAB-MMAE]₂ conjugate of example 75.

FIG. 59 A-H show HIC-HPLC of Trastuzumab-[MC-VC-PAB-MMAE]₂ conjugate of examples 76-83 (The parameters of step (1) and/step (2) is (are) different).

FIG. 60 A-G show HIC-HPLC of Trastuzumab-[MC-VC-PAB-MMAE]₂ conjugate of examples 84-90 (The parameters of step (1) and/step (2) is (are) different).

FIG. 61 A-C show HIC-HPLC of Trastuzumab-[MC-VC-PAB-MMAE]₂ conjugate of comparative examples 7-9 (The concentration of the transition metal ions is 0).

FIG. 62 A-D show HIC-HPLC of Trastuzumab-[MC-VC-PAB-MMAE]₂ conjugate of examples 91-94 (The oxidation time and temperature in step (2) are different).

FIG. 63 A-H show HIC-HPLC of Trastuzumab-[MC-VC-PAB-MMAE]₂ conjugate of examples 95-102 (The buffer system is different).

FIG. 64 A-H show HIC-HPLC of Trastuzumab-[MC-VC-PAB-MMAE]₂ conjugate of examples 103-110 (The buffer system is different).

FIG. 65 shows HIC-HPLC of Trastuzumab-[MC-VC-PAB-MMAE]₂ conjugate of comparative example 10.

FIG. 66 shows HIC-HPLC of Trastuzumab-[Maleimide-PEG4-N3-DBCO-MMAE]ᵢ conjugate of example 111.

FIG. 67 shows HIC-HPLC of Trastuzumab-[Maleimide-PEG4-N3-DBCO-MMAE]₁[MC-GGFG-DXd]₆ conjugate of example 112.

FIG. 68 shows HIC-HPLC of Trastuzumab-[Maleimide-PEG4-N3-DBCO-Cy3]₁[MC-VC-PAB-MMAE]₂ conjugate of example 113.

FIG. 69 A shows HIC-HPLC of Trastuzumab-[Maleimide-PEG4-N3-DBCO-Cy3]₁ conjugate of example 113; B-C show HIC-HPLC of Trastuzumab-[Maleimide-PEG4-N3-DBCO-Cy3]₁[MC-VC-PAB-MMAE]₄ conjugate of examples 114-115.

FIG. 70 A shows HIC-HPLC of Trastuzumab-[MC-VC-PAB-MMAE]₂ conjugate of example 116; B shows HIC-HPLC of Trastuzumab-[MC-VC-PAB-MMAE]₂[MC-GGFG-DXd]₄ conjugate of example 116.

FIG. 71 shows HIC-HPLC of Trastuzumab-[MC-GGFG-DXd]₂ conjugate of comparative example 11.

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “an antibody” includes a plurality of antibodies; reference to “a transition metal ion” includes mixtures of transition metal ions, and the like. In this application, the use of “or” means “and/or” unless stated otherwise.

Throughout this disclosure, unless the context requires otherwise, the words “comprise”, “comprises”, “comprising”, “contain”, “contains” and “containing” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

A mixture of antibody-drug conjugates will be generated by the conventional conjugation processes or the bio-conjugation process of the present disclosure. In general, one antibody molecule belonging to IgG1 or IgG4 subclass has 4 interchain disulfide bonds, each of which is formed with two —SH groups. The antibody molecule can be subjected to partial or complete reduction of one or more interchain disulfide bonds to form 2n (n is an integer selected from 1, 2, 3 or 4) reactive —SH groups. In accordance with the number of drugs coupling to a single antibody molecule, the different conjugates containing different number of drug molecules are denominated as D0, D1, D2, D3, D4, D6 and D8. And thus, the “homogeneity” of antibody-drug conjugates is used to describe the property of dominance of one specific type of antibody-drug conjugate (i.e., one type selected from D0, D1, D2, D3, D4, D6 and D8 conjugates) in one given mixture of antibody-drug conjugates.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, the average drug loading for an antibody-drug conjugate ranges from 1 to about 8; from about 2 to about 6; or from about 3 to about 5.

As used herein, the term “D0” refers to the ADC in which the number of drugs coupling to a single antibody molecule is zero.

As used herein, the term “D1” or “the ADC with D1” refers to the ADC in which one of the thiobridge group bearing the linker-payload re-bridges two thiol groups of one single antibody molecule.

As used herein, the term “D2” or “the ADC with D2” refers to the ADC in which two drug molecules are coupled to one single antibody molecule, where two drug molecules may be coupled to —SH groups generated by reduction of S—S bonds between heavy and light chains via linkers, or may be coupled to —SH groups generated by reduction of S—S bonds between heavy and heavy chains via linkers.

As used herein, the term “D3” or “the ADC with D3” refers to the ADC in which three of the thiobridge group bearing the linker-payload re-bridge six thiol groups of one single antibody molecule.

As used herein, the term “D4” or “the ADC with D4” refers to the ADC in which four drug molecules are coupled to one single antibody molecule, where four drug molecules may be coupled to four —SH groups generated by reduction of two S—S bonds between heavy and light chains via linkers, or four drug molecules may be coupled to four —SH groups generated by reduction of two S—S bonds between heavy and heavy chains via linkers, or two drug molecules may be coupled to two —SH groups generated by reduction of one S—S bond between heavy and light chains via linkers and the other two drug molecules may be coupled to two —SH groups generated by reduction of one S—S bond between heavy and heavy chains vis linkers.

As used herein, the term “D6” or “the ADC with D6” refers to the ADC in which six drug molecules are coupled to one single antibody molecule, where six drug molecules may be coupled to six-SH groups generated by reduction of three S—S bonds.

As used herein, the term “D8” or “the ADC with D8” refers to the ADC in which eight drug molecules are coupled to one single antibody molecule, where eight drug molecules may be coupled to eight-SH groups generated by reduction of four S—S bonds.

As used herein, the term “D6+D1” or “the bi-payload ADC with D6+D1” refers to the ADC in which six of the first linker-payloads and one of the second thiobridge groups bearing the second linker-payload are coupled to one single antibody molecule.

As used herein, the term “D6+D2’ or “the bi-payload ADC with D6+D2” refers to the ADC in which six of the first linker-payloads and two of the second linker-payloads are coupled to one single antibody molecule.

As used herein, the term “D3+D1” or “the bi-payload ADC with D3+D1” refers to the ADC in which three of the first thiobridge group bearing the first linker-payload and one of the second thiobridge groups bearing the second linker-payload re-bridge eight thiol groups of one single antibody molecule.

As used herein, the term “D3+D2” or “the bi-payload ADC with D3+D2” refers to the ADC in which three of the first thiobridge group bearing the first linker-payload re-bridge six thiol groups and two of the second linker-payloads are coupled to one single antibody molecule.

As used herein, the term “D0+D2” or “the ADC with D0+D2” refers to the ADC in which one, two or three of the first thiobridge group re-bridge six thiol groups and two of the second linker-payloads are coupled to one single antibody molecule, or refers to the ADC in which two, four or six of the end capping reagents and two of the second linker-payloads are coupled to one single antibody molecule.

As used herein, the term “D0+D1” or “the ADC with D0+D1” refers to the ADC in which three of the first thiobridge group re-bridges six thiol groups and one of the second thiobridge group bearing the linker-payload re-bridge two thiol groups of one single antibody molecule, or refers to the ADC in which six of the end capping reagents react with six thiol groups and one of the second thiobridge group bearing the linker-payload re-bridge two thiol groups of one single antibody molecule.

As used herein, the term “D1+D6” or “the bi-payload ADC with D1+D6” refers to the ADC in which one of the first thiobridge group bearing the first linker-payload re-bridging two thiol groups and six of the second linker-payloads are coupled to one single antibody molecule, wherein, the first linker-payload and the second linker-payload may be same or different.

As used herein, the term “D1+D2” or “the bi-payload ADC with D1+D2” refers to the ADC in which one of the first thiobridge group bearing the first linker-payload re-bridging two thiol groups and two of the second linker-payloads are coupled to one single antibody molecule, wherein, the first linker-payload and the second linker-payload may be same or different.

As used herein, the term “D1+D4” or “the bi-payload ADC with D1+D4” refers to the ADC in which one of the first thiobridge group bearing the first linker-payload re-bridging two thiol groups and four of the second linker-payloads are coupled to one single antibody molecule, wherein, the first linker-payload and the second linker-payload may be same or different.

As used herein, the term “D2+D4” or “the bi-payload ADC with D2+D4” refers to the ADC in which two of the first linker-payloads and four of the second linker-payloads are coupled to one single antibody molecule.

Method of preparing an antibody with site-specific modification On the one aspect, the present application provides a method of preparing antibody with site-specific modification, which characterized in that, the site-specific modification is that three interchain disulfide bonds within the antibody are reduced selectively, the method comprises that using tris (2-carboxyethyl) phosphine (TCEP) or salt thereof and transition metal ions together.

On the second aspect, the present application provides a method of preparing an antibody with site-specific modification, which characterized in that, the site-specific modification is that three interchain disulfide bonds within the antibody are reduced selectively, two of the three interchain disulfide bonds are in the Fab region and one is in the hinge region of the antibody, the method comprises that using TCEP or salt thereof and transition metal ions together.

As used herein, the term “disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain bridge or interchain bond.

As used herein, the term “hinge region” refers to an antibody includes the portion of a heavy chains molecule that joins the CH1 domain to the CH2 domain. This hinge region comprises approximately 25 amino acid residues and is flexible, thus allowing the two N-terminus antigen binding regions to move independently.

As used herein, the term “Fab fragments” refers to the region of the antibody structure that can bind to antigen. It consists of a complete light chain (variable and constant regions) and part of the heavy chain structure (variable and a constant region fragment), the light and heavy chains are connected by a disulfide bond. Fab fragments can be obtained by protease digestion of full-length antibodies. Under the action of papain, human immunoglobulin G can be degraded into two Fab fragments and one Fc fragment; under the action of pepsin, IgG can be degraded into an F(ab′)2 fragment and a pFc′ fragment. The F(ab′)2 fragment can be further reduced to form two Fab′ fragments. In some embodiments, the interchain disulfide bonds connect two of the upper heavy chains in the hinge region or the interchain disulfide bonds connect the heavy chain to the light chain in Fab region.

On the third aspect, the present application provides a method of preparing an antibody with site-specific modification, which characterized in that, the site-specific modification is that three interchain disulfide bonds within the antibody are reduced selectively, two of the three interchain disulfide bonds are in the Fab region and one is in the hinge region of the antibody, the method comprises the following steps:
(a) incubating TCEP or salt thereof and transition metal ions in the presence of an antibody in a buffer system to selectively reduce interchain disulfide bonds within the antibody to afford the antibody bearing reduced thiol groups, the molar ratio of TCEP and the antibody is 3:1-15:1, optionally, the molar ratio of TCEP and the antibody is 3:1-6:1;

In some embodiments, in step (a), three interchain disulfide bonds within the antibody are reduced selectively. The molar ratio of TCEP and the antibody is very important to selectively reduce three interchain disulfide bond.

In some embodiments, the salt thereof refers to acid addition salt or base addition salt.

In some embodiments, acid addition salts can be formed with inorganic acids and organic acids. The inorganic acids from which salts can be derived include, for example, hydrochloric acid, hydrobromic acid, sulfuric acid, phosphoric acid, and the like. The organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

In some embodiment, base addition salts can be formed with inorganic bases and organic bases. The inorganic bases from which salts can be derived include groups 1 to 2 of the periodic table. In certain embodiments, the salts are derived from lithium, sodium, potassium, calcium, magnesium and the like. The organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In some embodiments, in step (a), the molar ratio of TCEP and the antibody is 3.2:1 to 5:1 or 3.5:1 to 4.4:1.

The molar ratio of TCEP and the antibody is important to selectively reduce three interchain disulfide bonds within the antibody. In some embodiments, in step (a), the molar ratio of TCEP and the antibody is 3.1:1 to 5.5:1, 3.1:1 to 5.0:1, 3:1 to 4.8:1, 3.2:1 to 4.8, 3.4:1 to 4.8, 3.6:1 to 4.8 or 3.8:1 to 4.8. In some embodiments, in step (a), the molar ratio of TCEP and the antibody is 3:1 to 4.5:1 or 3:1 to 4:1. In some embodiments, in step (a), the molar ratio of TCEP and the antibody is 3.2:1 to 4.4:1.

In some embodiments, in step (a), the molar ratio of TCEP and the antibody is 3:1, 4:1, 5:1, 6:1, 7:1, 8:1, 9:1, 10:1, 11:1, 12:1, 13:1, 14:1 or 15:1.

In some embodiments, the incubation temperature is 0° C. to 37° C. in step (a), optionally, the incubation temperature is 0° C. to 25° C. in step (a), more optionally, the incubation temperature is 0° C. to 15° C. in step (a). In some embodiments, the incubation temperature is 0° C. to 10° C., 0° C. to 8° C., 0° C. to 6° C. in step (a). In some embodiments, the incubation temperature is 4° C. in step (a).

In some embodiments, the incubation temperature is 37° C., 35° C., 33° C., 30° C., 28° C., 24° C., 20° C., 18° C., 15° C., 13° C., 10° C., 8° C. or 4° C. in step (a).

The incubation time in step (a) is important to selectively reduce three interchain disulfide bonds within the antibody. In some embodiments, the incubation time is 3 h-24 h in step (a). In some embodiments, the incubation time is 12 h-24 h in step (a), optionally, the incubation time is 16 h to 20 h in step (a), more optionally, the incubation time is 16 h to 18 h in step (a).

In some embodiments, the incubation time is 4-24 h, 14 h-24 h or 16 h-24 h in step (a). In some embodiments, the incubation time is 17 h-18 h in step (a). In some embodiments, the incubation time is 4 h, 6 h, 8 h, 10 h, 12 h, 14 h, 16 h, 18 h, 20 h, 22 h or 24 h in step (a).

In some embodiments, the incubation temperature is 4° C. and the incubation time is 18 h in step (a).

In some embodiments, in step (a), the molar ratio of TCEP and the antibody is 3:1 to 6:1, the incubation time is 10 h to 24 h. In some embodiments, in step (a), the molar ratio of TCEP and the antibody is 3:1, 3.2:1, 3.5:1, 3.8:1, 4:1, 4.5:1, 5:1, 5.5:1 or 5:8:1, the incubation time is 10 h, 11 h, 12 h, 13 h, 14 h, 15 h, 16 h, 17 h, 18 h, 19 h, 20 h, 21 h, 22 h, 23 h or 24 h.

In some embodiments, the incubation time in step (a) is shortened with increasing the molar ratio of TCEP and the antibody. In some embodiments, in step (a), the molar ratio of TCEP and the antibody is 4:1 to 15:1, the incubation time is 4 h-12 h. In some embodiments, in step (a), the molar ratio of TCEP and the antibody is 7:1 to 15:1, the incubation time is 4 h-12 h. In some embodiments, in step (a), the molar ratio of TCEP and the antibody is 8:1 to 13:1, the incubation time is 4 h-10 h. In some embodiments, the molar ratio of TCEP and the antibody is 6.2:1, 6.5:1, 6.8:1, 7:1, 8:1, 9:1, 10:1, 11:1, 12:1, 13:1, 14:1 or 15:1, the incubation time is 4 h, 5 h, 6 h, 7 h, 8 h, 9 h, 10 h, 11 h or 12 h.

In some embodiments, in step (a), the molar ratio of the transition metal ions and TCEP is 0.1:1 to 30:1, optionally, the molar ratio of the transition metal ions and TCEP is 0.1:1 to 20:1, more optionally, the molar ratio of the transition metal ions and TCEP is 0.5:1 to 8:1. In some embodiments, the molar ratio of the transition metal ions and TCEP is 0.1:1 to 15:1, 0.1:1 to 10:1, 0.1:1 to 8:1, 0.25:1 to 15:1, 0.25:1 to 12:1, 0.25:1 to 10:1, 0.25:1 to 8:1, 0.25:1 to 7.5:1, 0.25:1 to 7:1, 0.25:1 to 5:1, 0.25:1 to 4:1 or 0.5:1 to 4:1.

In some embodiments, in step (a), the molar ratio of the transition metal ions and TCEP is 0.1:1, 0.2:1, 0.5:1, 0.8:1, 1:1, 2:1, 4:1, 8:1, 10:1, 12:1, 14:1, 16:1, 18:1 or 20:1.

In some embodiments, there is no specific limitation to the concentration of the TCEP, as long as scaling up or down the concentration of the transition metal ions and the antibody in equal proportions. In some embodiments of the present applications, the concentration of the first reductant is 0.01 mM to 0.2 mM. In some embodiments of the present applications, the concentration of the first reductant is 0.02 mM to 0.15 mM. In some embodiments of the present applications, the concentration of the first reductant is 0.05 mM to 0.1 mM. In some embodiments of the present applications, the concentration of the first reductant is 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM or 0.20 mM.

In some embodiments, there is no specific limitation to the concentration of the transition metal ions in step (a), as long as scaling up or down the concentration of TCEP and the antibody in equal proportions.

In some embodiments of the present application, there is no specific limitation to the concentration of the antibody in step (a), as long as scaling up or down the concentration of TCEP and the transition metal ions in equal proportions.

In some embodiments, the buffer system is selected from a group consisting of MES buffer, Bis-Tris buffer, PIPES buffer, MOPS buffer, BES buffer, HEPES buffer, DIPSO buffer, MOBS buffer, MOPSO buffer, TES buffer, ACES buffer, TAPSO buffer, PBS, PB, Acetate buffer, BTP buffer, HEPPSO buffer, POPSO buffer, EPPS buffer or Tris buffer.

As used herein, the term “MES buffer” refers to 2-(N-morpholino) ethanesulfonic acid buffer.

As used herein, the term “Bis-Tris buffer” refers to Bis(2-hydroxyethyl)amino-tris(hydroxymethyl)methane buffer.

As used herein, the term “PIPES buffer” refers to piperazine-1,4-bisethanesulfonic acid buffer.

As used herein, the term “MOPS buffer” refers to 3-morpholinopropanesulfonic Acid buffer.

As used herein, the term “BES buffer” refers to N, N-Bis (2-hydroxyethyl)-2-aminoethanesulphonic acid buffer.

As used herein, the term “HEPES buffer” refers to 4-hydroxyethyl piperazine ethanesulfonic acid buffer.

As used herein, the term “DIPSO buffer” refers to 3-[bis(2-hydroxyethyl)amino]-2-hydroxypropanesulphonic acid buffer.

As used herein, the term “MOBS buffer” refers to 3-morpholinopropanesulfonic Acid buffer.

As used herein, the term “MOPSO buffer” refers to 3-(N-morpholino)-2-hydroxy-1-propanesulfonic acid buffer.

As used herein, the term “TES buffer” refers to 2-[tris(hydroxymethyl)methylamino]-1-ethanesulfonic acid buffer.

As used herein, the term “ACES buffer” refers to N-(carbamoylmethyl)taurine buffer.

As used herein, the term “TAPSO buffer” refers to 3-[N-tris-(hydroxymethyl)methylamino]-2-hydroxypropanesulphonic acid buffer.

As used herein, the term “PBS” refers to phosphate buffer saline.

As used herein, the term “ADA buffer” refers to N-(Carbamoylmethyl) iminodiacetic acid buffer.

As used herein, the term “PB buffer” refers to refers to phosphate buffer.

As used herein, the term “BTP buffer” refers to Bis-tris propane buffer.

As used herein, the term “Heppso buffer” refers to N-(Hydroxyethyl) piperazine-N′-2-hydroxypropanesulfonicacid buffer.

As used herein, the term “POPSO buffer” refers to piperazine-N, N′-bis(2-hydroxy-propane sulfonic) acid buffer.

As used herein, the term “EPPS buffer” refers to 4-(2-Hydroxyethyl)-1-piperazinepropanesulfonic acid buffer.

As used herein, the term “Tris buffer” refers to tris(hydroxymethyl)aminomethane buffer.

In some embodiments, the buffer system is selected from a group consisting of MES buffer, Bis-Tris buffer, MOPS buffer, BES buffer, HEPES buffer, DIPSO buffer, MOBS buffer, MOPSO buffer, TES buffer, ACES buffer or TAPSO buffer.

In some embodiments, the buffer system is BES buffer.

In some embodiments, the pH value of the buffer system is 5.5 to 8.

In some embodiments, the pH value of the buffer system is 5.8 to 7.4, preferably, the pH value of the buffer system is 6.7 to 7.4. In some embodiments, the pH value of the system buffer is 6.0 to 7.4 or 6.4 to 7.4. In some embodiments, the pH value of the buffer system is 6.4, 6.7, 7.0 or 7.4.

In some embodiments, the buffer system is BES buffer and the pH value of BES buffer is 7.0. In some embodiments, the buffer system is BES buffer and the pH value of BES buffer is 6.4. In some embodiments, the buffer system is BES buffer and the pH value of BES buffer is 6.7. In some embodiments, the buffer system is BES buffer and the pH value of BES buffer is 7.4. In some embodiments, the buffer system is MES buffer and the pH value of BES buffer is 7.0.

In some embodiments, the concertation of the buffer system is 10 mM to 100 mM.

In some embodiments, the concertation of the buffer system is 20 mM to 80 mM, preferably, the concertation of the buffer system is 20 mM to 40 mM. In some embodiments, the concertation of the buffer system is 20 mM to 60 mM. In some embodiments, the concertation of the buffer system is 10 mM, 20 mM, 30 mM, 40 mM, 50 mM, 60 mM, 70 mM, 80 mM, 90 mM or 100 mM.

In some embodiments, the transition metal ions are selected from a group consisting of Zn²⁺, Cd²⁺, Ni²⁺, Hg²⁺, Mn²⁺, Co²⁺ and the combination thereof.

The term “transition metal ions” refers to the elements of groups 4-12, justified by their typical chemistry, i.e., a large range of complex ions in various oxidation states, colored complexes, and catalytic properties either as the element or as ions (or both). Sc and Y in Group 3 are also generally recognized as transition metals.

In some embodiments, the transition metal ions selected from a group consisting of Zn²⁺, Cd²⁺, Hg²⁺, Ni²⁺, Co²⁺ or the combination thereof.

In some embodiments, the transition metal ions are Zn²⁺.

In some embodiments, there is no specific limitation to the salts of the transition metal ions, as long as the transition metal ions are soluble in the reaction solution so that free transition metal ions can be released in the reaction solution. In some embodiments of the present application, the salts of the transition metal ions are chloride, nitrate, sulfate, acetate, iodide, bromine, formate or tetrafluorborate.

In some embodiments, the salts of Zn²⁺ are ZnCl₂, Zn(NO₃)₂, ZnSO₄, Zn(CH₃COO)₂, ZnI₂, ZnBr₂, Zinc formate, or zinc tetrafluoroborate. In some embodiments, the salts of Zn²⁺ are ZnCl₂.

In some embodiments, the site-specific modification dose not refer to antibody engineering, enzyme technologies and glycan modification.

In some embodiments, the method comprises introducing metal chelators after step (a).

In some embodiments, the metal chelators can trap excessive the transition metal ions, which is helpful to selectively reduce three interchain disulfide bonds within the antibody. In some embodiments, there is no specific limitation to the metal chelators, as long as the metal chelators can trap the excessive transition metal ions and do not affect the reduction of the disulfide bonds within the antibody.

In some embodiments, the metal chelators are selected from a group consisting of ethylene diamine tetraacetic acid (EDTA), nitrilotriacetic acid (NTA), diethylenetriaminepentaacetic acid (DTPA), citric Acid (CA), tartaric acid (TA), gluconic acid (GA) or N-(2-hydroxyethyl) ethylenediamine-N, N′, N′-triacetic acid (HEDTA).

In some embodiments, the metal chelators are selected from a group consisting of EDTA, NTA or DTPA. In some embodiments, the metal chelators are EDTA.

On the fourth aspect, the present application provides a method of preparing ADC with D2 or the ADC with D1, which characterized in that, the method comprises the method of the present application, and also comprises the following steps:
(B1) introducing oxidant to selectively re-oxidize the reduced thiol groups resulted from step (a), optionally, re-oxidize the reduced thiol groups in Fab region, preferably, removing the excessive oxidant to purify the oxidized products;
(C1) introducing the metal chelators and modification reagent 1 to react with the remained thiol groups resulted from step (B1), wherein, the modification reagent 1 is an end capping reagent, a first linker-payload or a first thiobridge reagent, optionally, the first thiobridge reagent bears the first linker-payload or reactive groups.

As used herein, the term “bears” or “bearing” refers to have or having.

In some embodiments, when the first thiobridge bears the reactive groups, the step (C1) comprises the following steps:
introducing the metal chelators and the first thiobridge reagent bearing the reactive groups to re-bridge the reduced thiol groups resulted from step (B1), then, incubating the first linker-payload in the buffer system to react with the reactive groups of the first thiobridge group.

In some embodiments, in step (a), the molar ratio of TCEP and the antibody is 4:1 to 15:1, the incubation time is 1 h-24 h. In some embodiments, in step (a), the molar ratio of TCEP and the antibody is 4:1 to 15:1, the incubation time is 1 h-16 h. In some embodiments, in step (a), the molar ratio of TCEP and the antibody is 4:1 to 10:1, the incubation is 4 h-8 h.

In some embodiments, in step (a), the concentration of the transition metal ions is also important to improve the reduction selectivity. In some embodiments, the method of preparing ADC with D2 comprising the following steps: in step (a), the molar ratio of the transition metal ions and the antibody is 1:1 to 10:1 or 1:1 to 2:1. In some embodiments, in step (a), the molar ratio of the transition metal ions and the antibody is 1:1 to 9:1, 1:1 to 8:1, 1:1 to 7:1, 1:1 to 6:1, 1:1 to 5:1:1, 1:1 to 4.5:1, 1:1 to 4:1, 1:1 to 3.5:1, 1:1 to 3:1 or 1:1 to 2.5:1.

In some embodiments, there is no specific limitation to the oxidant, as long as the oxidant can re-oxidize the reduced thiol groups. In some embodiments, the oxidant is Dehydroascorbic acid (DHAA).

In some embodiments, in step (B1), the oxidant selectively re-oxidizes the reduced thiol groups resulted from step (a), providing the antibody with two reduced thiol groups.

In some embodiments, in step (B1), the concentration of the oxidant is important to improve the oxidation selectivity. In some embodiments, in step (B1), the molar ratio of oxidant and the antibody is 2:1 to 25:1, optionally, in step (B1), the molar ratio of oxidant and the antibody is 2:1 to 20:1, more optionally, the molar ratio of oxidant and the antibody is 8:1 to 15:1. In some embodiments, in step (B1), the molar ratio of oxidant and the antibody is 2.5:1 to 15:1, 3:1 to 15:1, 3.5:1 to 15:1, 4:1 to 15:1, 4.5:1 to 15:1, 5:1 to 15:1, 5.5:1 to 15:1, 6:1 to 15:1 or 7:1 to 15:1.

In some embodiments, in step (B1), the molar ratio of the oxidant and the antibody is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1 or 25:1.

In some embodiments, in step (B1), the oxidation temperature is 0° C. to 37° C., the oxidation time is 1 h to 48 h. In some embodiments, in step (B1), the oxidation temperature is 0° C. to 30° C., the oxidation time is 1 h to 8 h.

In some embodiments, in step (B1) the oxidation temperature is 0° C. to 37° C., 0° C. to 25° C., 0° C. to 20° C., 0° C. to 10° C., 0° C. to 4° C. or 4° C. to 10° C. In some embodiments, the oxidation temperature is 0° C., 3° C., 6° C., 8° C., 10° C., 12° C., 15° C., 18° C., 20° C., 22° C., 25° C., 28° C., 30° C., 32° C., 35° C. or 37° C.

In some embodiments, in step (B1), the oxidation time is 1 h to 7 h, 1 h to 6 h, 1 h to 5 h, 1 h to 3 h, 2 h to 5 h, 2 h to 4 h or 2 h to 3 h. In some embodiments, the oxidation time is 1 h, 3 h, 5 h, 7 h, 9 h, 11 h, 13 h, 15 h, 18 h, 20 h, 23 h, 25 h, 27 h, 30 h, 33 h, 35 h, 37 h, 40 h, 43 h, 45 h or 48 h.

In some embodiments, in step (B1), the oxidation temperature is 25° C., the oxidation time is 1 h to 3 h. In some embodiments, the oxidation temperature is 0° C.-10° C., the oxidation time is 5 h to 8 h.

In some embodiments, in step (B1), the oxidation reaction is in darkness.

In some embodiments, in step (B1), it is significant to improve the content of the ADC with D2, the ADC with D4 and the ADC with D1 that removing the excessive oxidant to purify the oxidized products.

In some embodiments, in step (C1), the reaction temperature with the reduced thiol groups is 0° C. to 37° C., 0° C. to 30° C., 5° C. to 25° C., 10° C. to 25° C. or 14° C. to 25° C. In some embodiments, in step (C1), the reaction temperature with the reduced thiol groups is 0° C., 0° C., 3° C., 9° C., 13° C., 18° C., 20° C., 23° C., 25° C., 27° C., 29° C., 30° C., 33° C., 35° C. or 37° C.

In some embodiments, in step (C1), the reaction time with the reduced thiol groups is 1 h to 6 h, 1 h to 5 h, 1 h to 3 h, 1 h to 2 h or 1 h to 1.5 h. In some embodiments, in step (C1), the reaction time with the reduced thiol groups is 1 h, 2 h, 3 h, 4 h, 5 h or 6 h.

In some embodiments, in step (C1), the reaction temperature with the reduced thiol groups is 0° C. to 30° C., the reaction time with the reduced thiol groups is 1 h to 4 h. In some embodiments, in step (C1), the reaction temperature with the reduced thiol groups is 15° C. to 25° C., the reaction time with the reduced thiol groups is 1 h to 2 h.

In some embodiments, in step (C1), the reaction temperature with the reactive groups is 10° C. to 37° C., 20° C. to 30° C., 10° C. to 30° C., 15° C. to 30° C. or 25° C. to 30° C. In some embodiments, in step (b) and in step (d), the reaction temperature with the reactive groups is 10° C., 13° C., 15° C., 17° C., 20° C., 23° C., 25° C., 28° C., 30° C., 33° C., 35° C. or 37° C.

In some embodiments, in step (C1), the reaction time with the reactive groups is 2 h to 12 h, 2 h to 10 h, 4 h to 10 h, 6 h to 10 h or 8 h to 10 h. In some embodiments, in step (C1), the reaction time with the reactive groups is 2 h, 3 h, 5 h, 7 h, 9 h, 11 h or 12 h.

In some embodiments, in step (C1), according to the amount of the antibody, the modification reagent 1 is excess.

In some embodiments, in step (C1), the molar ratio of the first thiobridge reagent and the antibody is 5:1 to 1:1, 2:1 to 1:1, 1.5:1 to 1:1, 1.2:1 to 1:1 or 1.1:1 to 1:1. In some embodiment, in step (C1), the molar ratio of the first thiobrige reagent and the antibody is 1.05:1.

In some embodiments, in step (C1), when the first linker-payload reacts with the reactive groups in the first thiobridge reagent, the molar ratio of the first linker-payload and the antibody is 5:1 to 1:1, 4:1 to 1:1.1, 3:1 to 1:1 or 2:1 to 1:1. In some embodiments, in the step (C1), the molar ratio of the first linker-payload and the antibody is 5:3.

In some embodiments, in step (C1), when the first linker-payload reacts with the reduced thiol groups, the molar ratio of the first linker-payload and the antibody is 2:1 to 10:1, 3:1 to 10:1, 4:1 to 9:1 or 5:1 to 7:1. In some embodiments, in step (C1), when the first linker-payload reacts with the reduced thiol groups, the molar ratio of the first linker-payload and the antibody is 5:1.

In some embodiments, the method also comprises the following steps:
(D1) optionally, introducing a compound which contains at least one thiol group to consume excessive the first linker-payloads;
(E1) purifying and recovering the ADC with D2 or the ADC with D1.

In some embodiments, there is no specific limitation to a compound to consume excessive said linker-payload, as long as the compound contains at least one thiol group. In some embodiments of the present application, the compound is cysteine.

In some embodiments, the oxidized products in step (B1), the resultant ADC with D2 and/or the resultant ADC with D1 are purified by a de-salting column, size exclusion chromatography, ultrafiltration, dialysis and/or the like. In some embodiments, the oxidized products in step (B1), the resultant ADC with D2 and/or the resultant ADC with D1 are purified by a de-salting column.

In some embodiments, the method of preparing the ADC with D2 comprises step (a), (B1) and (C1), wherein, the modification reagent 1 is the first linker-payload.

In some embodiments, the method of preparing the ADC with D2 comprises the following steps:
(1) TCEP (4 eq-15 eq) and ZnCl₂ (1 eq-2 eq) were added to a solution of Trastuzumab (0.01 mM-0.2 mM) in BES buffer (20 mM, pH7.0) and the reaction mixture was vortexed for mixing, then the reaction mixture was incubated at 4° C. for 4-24 h;
(2) Adding DHAA (4 eq-20 eq) to selectively re-oxidize the reduced thiol groups in Fab region resulted from step (1), the mixture was incubated in darkness at 0° C. to 37° C. for 1 h to 48 h, preferably, removing the excessive DHAA to purify the oxidized products;
(3) Introducing EDTA (8 eq-120 eq) and MC-VC-PAB-MMAE (6 eq) in 10% v/v DMA to react with remained thiol groups resulted from step (2) and the reaction was incubated at 0° C. to 30° C. for 1 h to 4 h;
(4) The reaction mixture was subjected to purification using a de-salting column.

In some embodiments, the homogeneity of the ADC with D2 is up to 60%, 70%, 75%, even to 80%, 85%, 90% or 95%.

In some embodiments, the method of preparing the ADC with D1 comprises step (a), (B1) and (C1), wherein, the modification reagent 1 is the first thiobridge reagent bears the first linker-payload.

In some embodiments, the method of preparing the ADC with D1 comprises the following steps:
(1) TCEP (4 eq-15 eq) and ZnCl₂ (1 eq-2 eq) were added to a solution of Trastuzumab (0.01 mM-0.2 mM) in BES buffer (20 mM, pH7.0) and the reaction mixture was vortexed for mixing, then the reaction mixture was incubated at 4° C. for 4-24 h;
(2) Adding DHAA (4 eq-20 eq) to selectively re-oxidize the reduced thiol groups in Fab region resulted from step (1) at 0° C. to 37° C. for 1 h to 48 h;
(3) Introducing EDTA (8 eq-120 eq) and a first thiobridge reagent dibromomaleimide-PEG4-N3 (0.013 mM) to react with reduced thiol groups resulted from step (3), the reaction temperature is 4° C. to 37° C. and the reaction time is 1 h to 6 h, then recovering the product using a desalting column to afford Trastuzumab-[Maleimide-PEG4-N3]ᵢ;
(4) incubating Trastuzumab-[Maleimide-PEG4-N3]₁ and DBCO-MMAE (0.02 mM) in BES buffer (20 mM, pH7.0), the reaction temperature is 10° C. to 37° C. and the reaction time is 2 h to 12 h.
(5) The reaction mixture was subjected to purification using a de-salting column.

In some embodiments, the homogeneity of the ADC with D1 is up to 90%, even to 95%.

In some embodiments, the present application provides a method of preparing ADC with D4, the ADC with D2+D3, the ADC with D2+D1, the ADC with D1+D2, the ADC with D1+D4, the ADC with D2+D4, the ADC with D1+D6 and the ADC with D1+D3, the method comprises the following steps:
(C2) introducing the metal chelators and a second reductant to selectively reduce the antibody from step (B1), optionally, reduce the interchain disulfide bonds in the hinge region of the antibody;
or
(C2′) introducing the second reductant to reduce the interchain disulfide bonds in the product from step (C1), optionally, introducing the transition metal ions;
(D2) introducing the modification reagent 2 to react with the reduced thiol groups resulted from step (C2) or step (C2′), optionally, introducing the metal chelators, wherein, the modification reagent 2 is a second linker-payload or a second thiobridge reagent, optionally, the second thiobridge reagent bears the second linker-payload or reactive groups.

In some embodiments, when the second thiobridge reagent bears the reactive groups, the step (D2) comprises the following steps:
introducing the product from step (C2) or step (C2′) and the second thiobridge reagent bearing the reactive groups to re-bridge the reduced thiol groups resulted from step (C2) or step (C2′), optionally, introducing the metal chelators, then, incubating the second linker-payload in the buffer system to react with the reactive groups of the second thiobridge group.

In some embodiments, when introducing the transition metal ions in step (C2′), introducing the metal chelators to trap the excess transition metal ions in step (D2).

In some embodiments, there is no specific limitation to the second reductant, as long as the second reductant could reduce the interchain disulfide bonds within the antibody. In some embodiments, the second reductant is TCEP, Tris(3-hydroxypropyl)phosphine (THPP), or Dithiothreitol (DTT). In some embodiments, the second reductant is TCEP.

In some embodiments, in step (C2), the molar ratio of the metal chelators and the antibody is 2:1 to 120:1. In some embodiments, in step (C2), the molar ratio of the metal chelators and the antibody is 2:1 to 100:1. In some embodiments, in step (C2), the molar ratio of the metal chelators and the antibody is 2:1 to 80:1. In some embodiments, in step (C2), the molar ratio of the metal chelators and the antibody is 5:1 to 60:1. In some embodiments, in step (C2), the molar ratio of the metal chelators and the antibody is 10:1 to 60:1. In some embodiments, in step (C2), the molar ratio of the metal chelators and the antibody is 20:1 to 60:1. In some embodiments, in step (C2), the molar ratio of the metal chelators and the antibody is 30:1 to 60:1. In some embodiments, in step (C2), the molar ratio of the metal chelators and the antibody is 40:1 to 60:1.

In some embodiments, in step (C2), the molar ratio of the metal chelators and the antibody is 50:1 to 60:1.

In some embodiments, in step (C2), the molar ratio of the second reductant and the antibody is 1:1 to 2:1. In some embodiments, in step (C2), the molar ratio of the second reductant and the antibody is 1.2:1 to 1.8:1. In some embodiments, in step (C2), the molar ratio of the second reductant and the antibody is 1:1 to 1.6:1. In some embodiments, in step (C2), the molar ratio of second reductant and the antibody is 1:1 to 1.4:1.

In some embodiments, in step (C2), the reduction temperature is 0° C. to 30° C., the reduction time is 1 h to 8 h. In some embodiments, in step (C2), the reduction temperature is 0° C. to 37′C, 5° C. to 25° C., 10° C. to 20′C or 10° C. to 15° C.

In some embodiments, in step (C2), the reduction time is 1 h to 8 h, 1 h to 7 h, 1 h to 6 h, 1 h to 5 h, 2 h to 5 h, 2 h to 4 h or 2 h to 3 h.

In some embodiments, in step (C2′), three of the interchain disulfide bonds in the product prepared from step (C1) are reduced completely without the transition metal ions. In some embodiments, one interchain disulfide bond or two interchain disulfide bonds in the product prepared from step (C1) is(are) reduced with the transition metal ions.

In some embodiments, in step (C2′), without the transition metal ions, there is no specific limitation to concentration of the second reductant, as long as the second reductant could reduce the interchain disulfide bonds within the antibody completely. In some embodiments, in step (C2′), the molar ratio of the second reductant and the antibody is 3:1 to 20:1, 3:1 to 10:1, 4:1 to 10:1, 5:1 to 9:1, 6:1 to 9:1, 6:1 to 8:1. In some embodiments, in step (C2′), the molar ratio of the second reductant and the antibody is 20:3.

In some embodiments, introducing the transition metal ions, two of the interchain disulfide bonds are selectively reduced. In some embodiments, in step (C2′), the molar ratio of the second reductant and the transition metal ions is 1:0.05 to 1:40, and/or the molar ratio of the second reductant and the antibody is 2.5:1 to 20:1, and/or the incubation time is 1 h to 24 h. In some embodiments, in step (C2′), the molar ratio of the second reductant and the transition metal ions is 1:0.05, 1:0.08, 1:0.1, 1:0.2, 1:0.3, 1:0.4, 1:0.5, 1:0.6, 1:0.7, 1:0.8, 1:0.9, 1:1, 1:2, 1:4, 1:6, 1:8, 1:10, 1:12, 1:14, 1:16, 1:18 or 1:20. In some embodiments, in step (C2′), the molar ratio of the second reductant and the antibody is 2.5:1, 3:1, 5:1, 7:1, 9:1, 11:1, 13:1, 15:1, 17:1, 19:1 or 20:1. In some embodiments, in step (C2′), the incubation time is 1 h, 2 h, 4 h, 6 h, 8 h, 10 h, 12 h, 14 h, 16 h, 18 h, 20 h, 22 or 24 h. In some embodiments, in step (C2′), the molar ratio of the second reductant and the transition metal ions is 1:0.05 to 1:40, and/or the molar ratio of the second reductant and the antibody is 3:1 to 15:1, and the incubation time is 1 h to 12 h. In some embodiments, in step (C2′), the molar ratio of the second reductant and the transition metal ions is 1:0.05 to 1:40, and/or the molar ratio of the second reductant and the antibody is 2.5:1 to 15:1, and the incubation time is 12 to 24 h.

In some embodiments, introducing the transition metal ions, one of the interchain disulfide bonds are selectively reduced. In some embodiments, in step (C2′), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h. In some embodiments, in step (C2′), the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, in step (C2′), the molar ratio of the second reductant and the antibody is 0.8:1, 1:1, 1.2:1, 1.4:1, 1.6:1, 1.8:1, 2:1, 2.2:1, 2.4:1, 2.5:1. In some embodiments, in step (C2′), the incubation time is 0.2 h, 1 h, 2 h, 4 h, 6 h, 8 h, 10 h, 12 h, 14 h, 16 h, 18 h, 20 h, 22 or 24 h. In some embodiments, in step (c), the molar ratio of the second reductant and the transition metal ions is 1:0.5 to 1:100, and/or the molar ratio of the second reductant and the antibody is 0.8:1 to 2:1, and the incubation time is 0.5 h to 24 h. In some embodiments, in step (C2′), the molar ratio of the second reductant and the transition metal ions is 1:0.5 to 1:100, and/or the molar ratio of the second reductant and the antibody is 2:1 to 2.5:1, and the incubation time is 1 h to 9 h.

In some embodiments, in step (C2′), the incubation temperature of the second reductant is 0° C. to 37° C., or 5° C. to 30° C. In some embodiments, in step (C2′), the incubation temperature of the second reductant is 10° C. to 30° C., 15° C. to 30° C., 20° C. to 30° C., or 25° C. to 30° C. In some embodiments, in step (C2′), the incubation temperature of the second reductant is 25° C.

In some embodiments, in step (C2′), the incubation time of the second reductant is 0.5 h to 24 h, or 5 h to 20 h. In some embodiments, in step (C2′), the incubation time of the second reductant is 6 h to 18 h, 8 h to 18 h, 8 h to 15 h or 8 h to 12 h. In some embodiments, in step (C2′), the incubation time of the second reductant is 3 h, 8 h, 12 h or 18 h.

In some embodiments, the molar ratio of the metal chelators and the antibody in step (C2′) is 1:1 to 100:1, 10:1 to 100:1, 20:1 to 100:1, 20:1 to 80:1, 20:1 to 70:1, 30:1 to 60:1, 40:1 to 50:1, 35:1 to 60:1, 40:1 to 55:1.

In some embodiments, the reaction temperature and time with the reduced thiol groups in step (D2) are same as that in step (C1). In some embodiments, the reactive temperature and time with the thiol groups in step (D2) and in step (C1) are independent.

In some embodiments, the reaction temperature and time with the reactive groups in step (D2) are same as that in step (C1). In some embodiments, the reactive temperature and time with the reactive groups in step (D2) and in step (C1) are independent.

In some embodiments, in step (D2), according to the amount of the antibody, the modification reagent 2 is excess.

In some embodiments, in step (D2), the molar ratio of the second thiobridge reagent and the antibody is 10:1 to 1:1, 5:1 to 1:1, 5:1 to 3:1, 4:1 to 3:1, 4:1 to 3.2:1 or 4:1 to 3.5:1. In some embodiments, in step (D2), the molar ratio of the second thiobridge reagent and the antibody is 10:1, 9:1, 8:1, 7:1, 6:1, 5:1, 4.5:1, 4:1, 3.8:1, 3.5:1, 3.2:1, 2:1 or 1:1.

In some embodiments, in step (D2), when the second linker-payload reacts with the reactive groups in the second thiobridge reagent, the molar ratio of the second linker-payload and the antibody is 10:1 to 1:1, 10:1 to 3:1, 9:1 to 3:1, 8:1 to 3:1, 7:1 to 3:1, 6:1 to 3:1, 5:1 to 3:1 or 4:1 to 3:1.

In some embodiments, in step (D2), when the second linker-payload reacts with the reduced thiol groups, the molar ratio of the second linker-payload and the antibody is 20:1 to 2:1, 20:1 to 6:1, 18:1 to 8:1, 16:1 to 8:1, 14:1 to 8:1, 12:1 to 10:1. In some embodiments, in step (D2), when the second linker-payload reacts with the reduced thiol groups, the molar ratio of the second linker-payload and the antibody is 35:3.

In some embodiments, the method of preparing ADC with D4 also comprises that purify the product from step (B1).

In some embodiments, the product from step (B1) is purified by a de-salting column, size exclusion chromatography, ultrafiltration, dialysis and/or the like. In some embodiments of the present application, the product from step (B1) is purified by a de-salting column.

In some embodiments, the method of preparing the antibody with site-specific modifications also comprises the following steps:
(E2) optionally, introducing a compound which contains at least one thiol group to consume excessive the linker-payload;
(F2) purifying and recovering the antibody with site-specific modifications.

In some embodiments, the details of the step (E2) and the step (F2) are similar to the step (D1) and the step (E1).

In some embodiments, the method of preparing the ADC with D4 comprises the step (a), the step (B1), the step (C2) and the step (D2), wherein, the modification reagent 2 is the second linker-payload.

In some embodiments, the method of preparing the ADC with D4 comprises the following steps:
(1) TCEP (4 eq-15 eq) and ZnCl₂ (1 eq-2 eq) were added to a solution of Trastuzumab (0.01 mM-0.2 mM) in BES buffer (20 mM, pH7.0) and the reaction mixture was vortexed for mixing, then the reaction mixture was incubated at 4° C. for 14-24 h;
(2) Adding DHAA (2 eq-20 eq) to selectively re-oxidize the reduced thiol groups in Fab region resulted from step (1), the mixture was incubated in darkness at 0° C. to 37° C. for 1 h to 48 h, then the mixture was subjected to purification using a de-salting column;
(3) Introducing EDTA (2 eq-120 eq) and TCEP (l eq-2 eq) to selectively reduce the interchain disulfide bond remained in the hinge region of the antibody from step (2), the reduction temperature is 0° C. to 30° C. and the reduction time is 1 h to 5 h;
(4) Introducing MC-VC-PAB-MMAE (3 eq-6 eq) in 10% v/v DMA to react with remained thiol groups resulted from step (3) and the reaction was incubated at 0° C. to 30° C. for 1 h to 4 h;
(5) The reaction mixture was subjected to purification using a de-salting column.

In some embodiments, the method of preparing the ADC with D1+D6 comprises the sept (a), the step (B1), the step (C1), the step (C2′) and the step (D2), wherein, the modification reagent 1 is the first thiobridge reagent, the modification reagent 2 is the second linker-payload and without the transition metal ions in step (C2′).

In some embodiments, the method of preparing the ADC with D1+D6 comprises the following steps,
(1) introducing the ADC with D1 according to the present application and TCEP (0.08 mM) in BES buffer (20 mM, pH7.0), the reaction temperature is 25° C. and the reaction time is 12 h;
(2) introducing MC-GGFG-DXd (0.14 mM) to solution from step (1), and the reaction mixture was allowed to stay at 24° C. for 1 h, then recovering Trastuzumab-[Maleimide-PEG4-N3-DBCO-MMAE]₁[MC-GGFG-DXd]₆ using a desalting column.

In some embodiments, the homogeneity of the ADC with D1+D6 is up to 70%, 75%, even to 80% or 85%.

In some embodiments, the method of preparing the ADC with D1+D2 comprises the step (a), the step (B1), the step (C1), the step (C2′) and the step (D2), wherein, the modification reagent 1 is the first thiobridge reagent bearing the first linker-payload, the modification reagent 2 is the second linker-payload and introducing the transition metal ions in step (C2′).

In some embodiments, the method of preparing the ADC with D1+D2 comprises the step (a), the step (B1), the step (C1), the step (C2′) and the step (D2), wherein, the modification reagent 1 is the first thiobridge reagent bearing reactive groups which reacts with the first linker-payload, the modification reagent 2 is the second linker-payload and introducing the transition metal ions in step (C2′).

In some embodiments, the content of the ADC with D1+D2 is up to 65%, 68%, 70%, even to 71% or 75%.

In some embodiments, the method of preparing the ADC with D1+D4 comprises the step (a), the step (B1), the step (C1), the step (C2′) and the step (D2), wherein, the modification reagent 1 is the first thiobridge reagent bearing the first linker-payload, the modification reagent 2 is the second linker-payload and introducing the transition metal ions in step (C2′).

In some embodiments, the method of preparing the ADC with D1+D4 comprises the step (a), the step (B1), the step (C1), the step (C2′) and the step (D2), wherein, the modification reagent 1 is the first thiobridge reagent bearing reactive groups which reacts with the first linker-payload, the modification reagent 2 is the second linker-payload and introducing the transition metal ions in step (C2′).

In some embodiments, the content of the ADC with D1+D4 is up to 70%, 75%, even to 80% or 83%.

In some embodiments, the method of preparing the ADC with D2+D4 comprises the step (a), the step (B1), the step (C1), the step (C2′) and the step (D2), wherein, the modification reagent 1 is the first linker-payload, the modification reagent 2 is the second linker-payload and introducing the transition metal ions in step (C2′).

In some embodiments, the content of the ADC with D2+D4 is up to 75%, 80%, 85%, even to 90%.

On the fifth aspect, the present application provides a method of preparing the antibody with site-specific modification, which characterized in that, the method comprises the method of the present application, and also comprises the following steps:
(b) introducing the metal chelators and the modification reagent 1 to react with the reduced thiol groups resulted from step (a).

In some embodiments, when the first thiobridge reagent bears the reactive groups, the step (b) comprises the following steps:
introducing the metal chelators and the first thiobridge reagent bearing reactive groups to re-bridge reduced thiol groups resulted from step (a), then, incubating the first linker-payload in the buffer system to react with the reactive groups of the first thiobridge group.

In some embodiments, the method also comprises the following step: purifying and recovering the product from step (b).

In some embodiments, the method further comprises the following steps,
(c) incubating the reaction product from (b) and the second reductant in the buffer system to reduce the interchain disulfide bonds in the reactive product from (b);
(d) introducing the incubation product from step (c) and the modification reagent 2 to react with the reduced thiol groups resulted from step (c).

In some embodiments, when the second thiobridge reagent bears the reactive groups, the step (d) comprises the following steps:
introducing product from step (c) and the second thiobridge reagent bearing reactive groups to re-bridge reduced thiol groups resulted from step (c), then, incubating the second linker-payload in the buffer system to react with the reactive groups of the second thiobridge group.

In some embodiments, the method also comprises the following step: purifying and recovering the product from (d).

In some embodiments, the method also comprises the following step: introducing a compound which contains at least one thiol group to consume the excessive first linker-payload and the excessive second linker-payload. In some embodiments, the compound is same as the compound in the (D1).

In some embodiments, the resultant antibody-drug conjugates are recovered by any suitable purification method, such as using a de-salting column, size exclusion chromatography, ultrafiltration, dialysis, ultrafiltration (UF)-diafiltration (DF), and the like. If needed, further ADC enrichment (e.g., D2) may be applied in some case using hydrophobic interaction chromatography (HIC).

In some embodiments, in step (d), the resultant ADC is purified by a desalting column, size exclusion chromatography, ultrafiltration, dialysis and/or the like. In some embodiments of the present application, in step (d), the resultant ADC is purified by a desalting column.

In some embodiments, in step (a), the molar ratio of TCEP and the antibody is 7:1 to 15:1, the incubation is 4 h to 12 h. In some embodiments, in step (a), the molar ratio of TCEP and the antibody is 8:1 to 14:1, the incubation time is 4 h to 10 h.

In some embodiments, in step (a), TCEP reduces the three interchain disulfide bond within the antibody selectively with the transition metal ions and the suitable molar ratio of TCEP and the antibody, optionally, in step (c), the second reductant reduces the remaining one interchain disulfide bonds. The antibody with site-specific modifications, such as the ADC with D6 or the ADC with D3, could be prepared by the method including the step (a) and (b). The antibody with site-specific modifications, such as the ADC with D6+D1, the ADC with D3+D1, the ADC with D6+D2, the ADC with D3+D2, the ADC with D0+D1, or the ADC with D0+D2, could be prepared by the method including the step (a), (b), (c) and (d).

In some embodiments, in step (b) and in step (d), the temperature of reaction with the reduced thiol groups is 4° C. to 37° C., the time of reaction with the reduced thiol groups is 0.5 h to 20 h.

In some embodiments, in step (b) and in step (d), the temperature of reaction with the reduced thiol groups is 20° C. to 30° C. or 20° C. to 25° C. In some embodiments, in step (b) and in step (d), the temperature of reaction with the reduced thiol groups is room temperature. In some embodiments, in step (b) and in step (d), the temperature of reaction with the reduced thiol groups is 4° C., 6° C., 8° C., 10° C., 13° C., 17° C., 20° C., 23° C., 27° C., 30° C., 34° C. or 37° C. As used herein, the term “room temperature” refers to 23° C.±2° C., 25° C.±5° C. or 20° C.±5° C.

In some embodiments, in step (b) and in step (d), the time of reaction with the reduced thiol groups is 0.5 h to 6 h, 0.5 h to 4 h, 0.5 h to 2 h, 1 h-2 h or 0.5 h to 1 h. In some modifications, in step (b) and in step (d), the time of reaction with the reduced thiol groups is 0.5 h, 1 h, 2 h, 3 h, 4 h 5 h, 6 h, 8 h, 10 h, 12 h, 14 h, 16 h, 18 h or 20 h.

In some embodiments, the temperature and time of reaction with the reduced thiol groups in step (b) and step (d) are independent.

In some embodiments, in step (b) and in step (d), the temperature of reaction with the reactive groups is 10° C. to 37° C., the time of reaction with the reduced thiol groups is 2 h to 12 h.

In some embodiments, in step (b) and in step (d), the temperature of reaction with the reactive groups is 10° C. to 30° C., 15° C. to 30° C. or 25° C. to 30′C. In some embodiments, in step (b) and in step (d), the temperature of reaction with the reactive groups is 4° C., 6° C., 8° C., 10° C., 13° C., 17° C., 20° C., 23° C., 27° C., 30° C., 34° C., 35° C. or 37° C.

In some embodiments, in step (b) and in step (d), the time of reaction with the reactive groups is 2 h to 10 h, 4 h to 10 h, 8 h to 10 h. In some embodiments, in step (b) and in step (d), the time of reaction with the reactive groups is 2 h, 3 h, 4 h, 5 h, 6 h, 7 h, 8 h, 9 h, 10 h, 11 h, 12 h.

In some embodiments, the temperature and time of reaction with the reactive groups in step (b) and step (d) are independent.

In some embodiments, in step (b), according to the amount of the antibody, the modification reagent 1 is excess.

In some embodiments, in step (b), the molar ratio of the first thiobridge reagent and the antibody is 3:1 to 15:1. In some embodiment, in step (b), the molar ratio of the firs thiobrige reagent and the antibody is 3:1, 3.3:1, 4:1, 5:1, 6:1, 7:1, 8:1, 9:1, 10:1 or 13:1.

In some embodiments, in step (b), when the first linker-payload reacts with the reactive groups in the first thiobridge reagent, the molar ratio of the first linker-payload and the antibody is 3:1 to 15:1. In some embodiments, in the step (b), when the first linker-payload reacts with the reactive groups in the first thiobridge reagent, the molar ratio of the first linker-payload and the antibody is 3:1, 4:1, 5:1, 6:1, 7:1, 8:1, 9:1, 10:1 or 13:1.

In some embodiments, in step (b), when the first linker-payload reacts with the reduced thiol groups, the molar ratio of the first linker-payload and the antibody is 6:1 to 20:1. In some embodiments, in step (b), when the first linker-payload reacts with the reduced thiol groups, the molar ratio of the first linker-payload and the antibody is 6:1, 20:3, 7:1, 8:1, 9:1, 10:1, 15:1 or 20:1.

In some embodiments, in step (d), according to the amount of the antibody, the modification reagent 2 is excess.

In some embodiments, in step (d), the molar ratio of the second thiobridge reagent and the antibody is 1:1 to 3:1. In some embodiments, in step (b), the molar ratio of the second thiobridge reagent and the antibody is 1:1, 1.5:1, 2:1 or 3:1.

In some embodiments, in step (d), when the second linker-payload reacts with the reactive groups in the second thiobridge reagent, the molar ratio of the second linker-payload and the antibody is 1:1 to 8:1. In some embodiments, in step (d), when the second linker-payload reacts with the reactive groups in the second thiobridge reagent, the molar ratio of the second linker-payload and the antibody is 1:1, 2:1, 3:1, 4:1, 5:1, 6:1, 7:1 or 8:1.

In some embodiments, in step (d), when the second linker-payload reacts with the reduced thiol groups, the molar ratio of the second linker-payload and the antibody is 2:1 to 16:1. In some embodiments, in step (d), when the second linker-payload reacts with the reduced thiol groups, the molar ratio of the second linker-payload and the antibody is 2:1, 3:1, 4:1, 5:1, 6:1, 7:1, 8:1, 10:1, 12:1, 14:1 or 16:1.

In some embodiments, in step (c), there is no specific limitation to the second reductant, as long as the second reductant could reduce the interchain disulfide bonds within the antibody. In some embodiments, in step (c), the second reductant is TCEP, Tris(3-hydroxypropyl)phosphine (THPP), or Dithiothreitol (DTT). In some embodiments, the second reductant is TCEP.

In some embodiments, in step (c), there is no specific limitation to the concentration of the second reductant, as long as the second reductant could reduce the interchain disulfide bonds within the antibody. In some embodiments, the molar ratio of the second reductant and the antibody is 1:1 to 20:1. In some embodiments, the molar ratio of the second reductant and the antibody is 1:1, 2:1, 4:1, 6:1, 8:1, 10:1, 12:1, 14:1, 16:1, 18:1 or 20:1.

In some embodiments, in step (c), the incubation temperature of the second reductant is 0° C. to 37° C., the incubation time is 0.5 h to 24 h.

In some embodiments, in step (c), the incubation temperature of the second reductant is 5° C. to 30° C., 10° C. to 30° C., 15° C. to 30° C., 20° C. to 30° C. or 25° C. to 30° C. In some embodiments, in step (c), the incubation temperature of the second reductant is 25° C.

In some embodiments, in step (c), the incubation time is 1 h to 20 h, 5 h to 20 h, 6 h to 18 h, 8 h to 18 h, 8 h to 15 h or 8 h to 12 h. In some embodiments, in step (c), the incubation time is 1 h, 2 h, 3 h, 4 h, 5 h, 6 h, 7 h, 8 h or 12 h.

In some embodiments, the method of preparing the ADC with D6 comprises the following steps:
(a) incubating TCEP or salt thereof and the transition metal ions in the presence of the antibody in the buffer system to selectively the reduce interchain disulfide bonds within the antibody to afford the antibody bearing reduced thiol groups, the molar ratio of TCEP and the antibody is 3:1 to 15:1;
(b1) introducing the metal chelators and the first linker-payload to react with the reduced thiol groups resulted from step (a).

In some embodiments, the homogeneity of the ADC with D6 is up to 55%, 65%, 70%, 80%, 85%, even to 90%.

In some embodiments, the method of preparing the bi-payload ADC with D6+D2 comprises the following steps:
(c2) incubating the ADC with D6 and the second reductant in the buffer system to reduce the interchain disulfide bonds within the ADC with D6;
(d2) introducing the incubation product from step (c2) and the second linker-payload to react with the reduced thiol groups resulted from step (c2).

In some embodiments, the homogeneity of the bi-payload ADC with D6+D2 is up to 80%, 85%, even to 90%.

In some embodiments, the method of preparing the bi-payload ADC with D6+D1 comprises the following steps:
(d3) introducing the incubation product from step (c2) and the second thiobridge reagent bearing the second linker-payload to react with the reduced thiol groups resulted from step (c2).

In some embodiments, the method of preparing the bi-payload ADC with D6+D1 comprises the following steps:
(d3′) introducing the incubation product from step (c2) and the second thiobridge reagent bearing reactive groups to re-bridge the reduced thiol groups resulted from step (c2), then, incubating the second linker-payload in the buffer system to react with the reactive groups of the second thiobridge group.

In some embodiments, the homogeneity of the bi-payload ADC with D6+D1 is up to 80%, 85%, even to 90%.

In some embodiments, the method of preparing the ADC with D3 comprises the following steps:
(b4) introducing the metal chelators and the first thiobridge reagent bearing the first linker-payload to react with the reduced thiol groups resulted from step (a).

In some embodiments, the method of preparing the ADC with D3 comprises the following steps:
(b4′) introducing the metal chelators and the first thiobridge reagent bearing reactive groups to re-bridge the reduced thiol groups resulted from step (a), then, incubating the first linker-payload in the buffer system to react with the reactive groups of the first thiobridge group.

In some embodiments, the homogeneity of the ADC with D3 is up to 80%, 82%, even up to 86%.

In some embodiments, the method of preparing the bi-payload ADC with D3+D2 comprises the following steps:
(c5) incubating the ADC with D3 and the second reductant in the buffer system to reduce the interchain disulfide bonds within the ADC with D3;
(d5) introducing the incubation product from step (c5) and the second linker-payload to react with the reduced thiol groups resulted from step (c5).

In some embodiments, the method of preparing the bi-payload ADC with D3+D1 comprises the following steps:
(d6) introducing the incubation product from step (c5) and the second thiobridge reagent bearing the second linker-payload to react with the reduced thiol groups resulted from step (c5).

In some embodiments, the method of preparing the bi-payload ADC with D3+D1 comprises the following steps:
(d6′) introducing the incubation product from step (c5) and the second thiobridge reagent bearing reactive groups to re-bridge the reduced thiol groups resulted from step (c5), then, incubating the second linker-payload in the buffer system to react with the reactive groups of the second thiobridge group.

In some embodiments, the method of preparing the ADC with D0+D2 comprises the following steps:
(b7) introducing the metal chelators and the first thiobridge reagent to react with the reduced thiol groups resulted from step (a);
(c7) incubating the reactive product from (b7) and the second reductant in the buffer system to reduce the interchain disulfide bonds within the product from (b7);
(d7) introducing the incubation product from step (c7) and the second linker-payload to react with the reduced thiol groups resulted from step (c7).

In some embodiments, the homogeneity of the ADC with D0+D2 is up to 60%, 65%, even up to 68% or 70%.

In some embodiments, the method of preparing the ADC with D0+D1 comprises the following steps:
(d8) introducing the incubation product form step (c7) and the second thiobridge reagent bearing the second linker-payload to react with the reduced thiol groups resulted from step (c7).

In some embodiments, the method of preparing the ADC with D0+D1 comprises the following steps:
(d8′) introducing the incubation product from step (c7) and the second thiobridge reagent bearing reactive groups to re-bridge the reduced thiol groups resulted from step (c7), then, incubating the second linker-payload in the buffer system to react with the reactive groups of the second thiobridge group.

In some embodiments, the homogeneity of the ADC with D0+D1 is up to 85%, 87%, even up to 90% or 92%.

In some embodiments, the first thiobridge reagent and the second thiobridge reagent independently contain at least two substituted groups allowing a re-bridging of the thiol groups.

In some embodiments, without the limitation, the first thiobridge reagent and the second thiobridge reagent are selected from the group consisting of

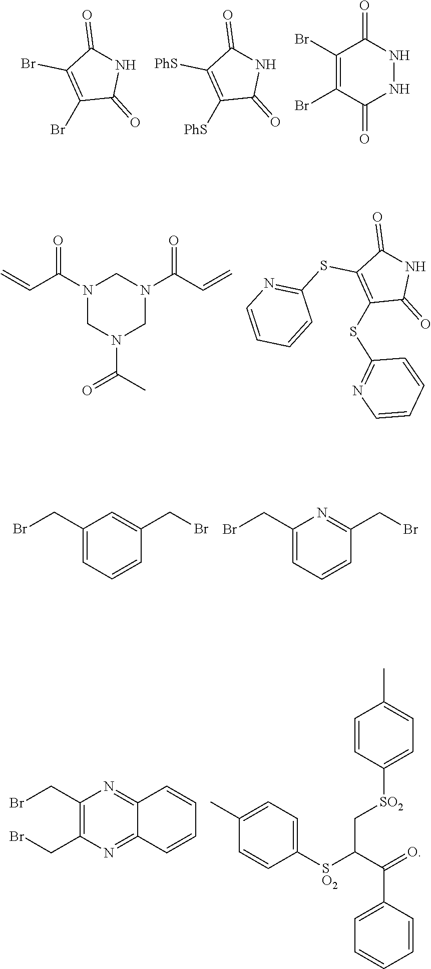

In some embodiments, the reactive groups independently contain azido and/or dibenzocyclooctyne (DBCO).

In some embodiments, the thiobridge reagent and the reactive groups are connected by alkyl group or polyethylene glycol (PEG).

In some embodiments, without the limitation, the first thiobridge reagent bearing reactive groups and the second thiobridge reagent bearing reactive groups are selected from the groups consisting of

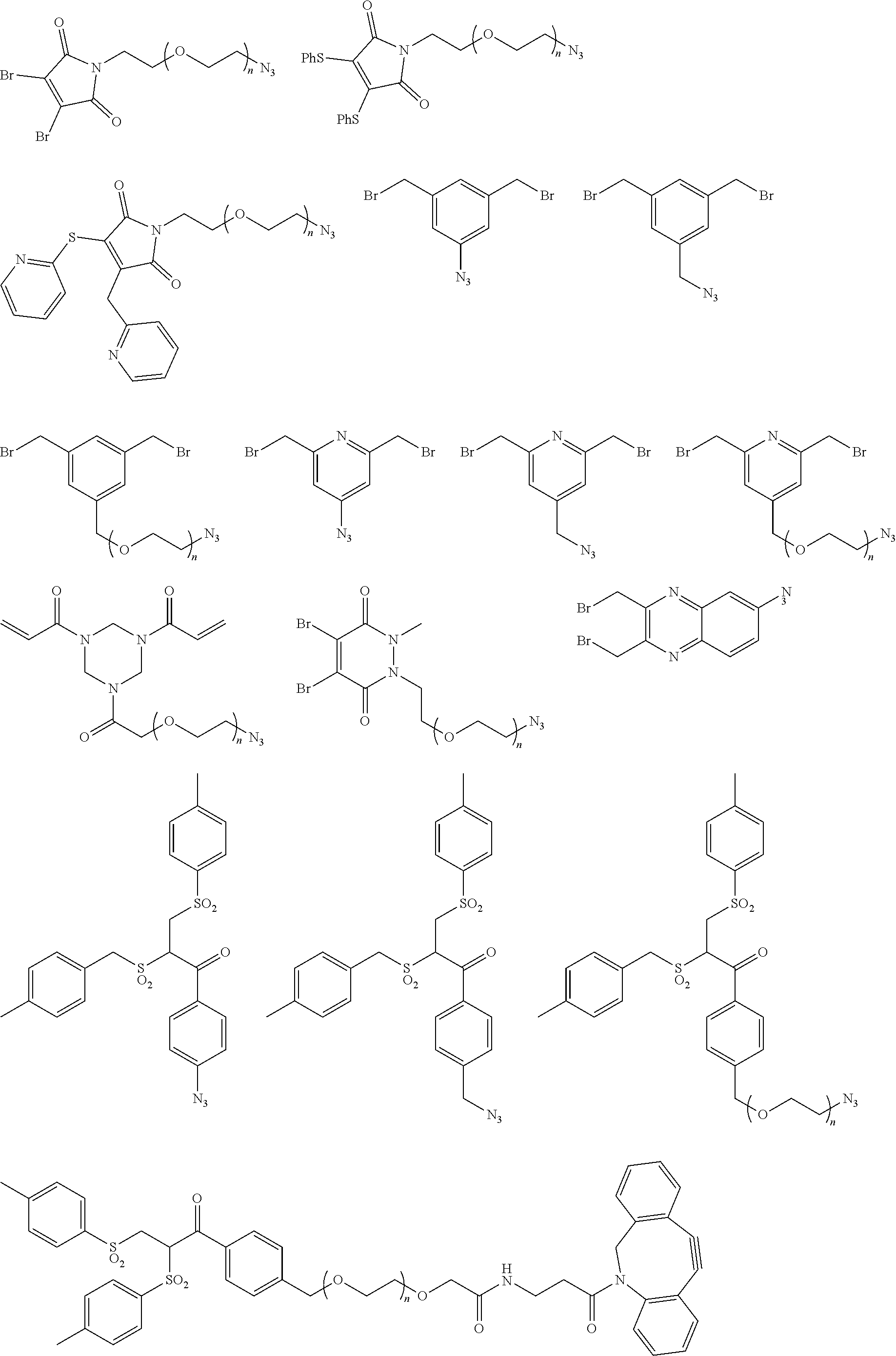

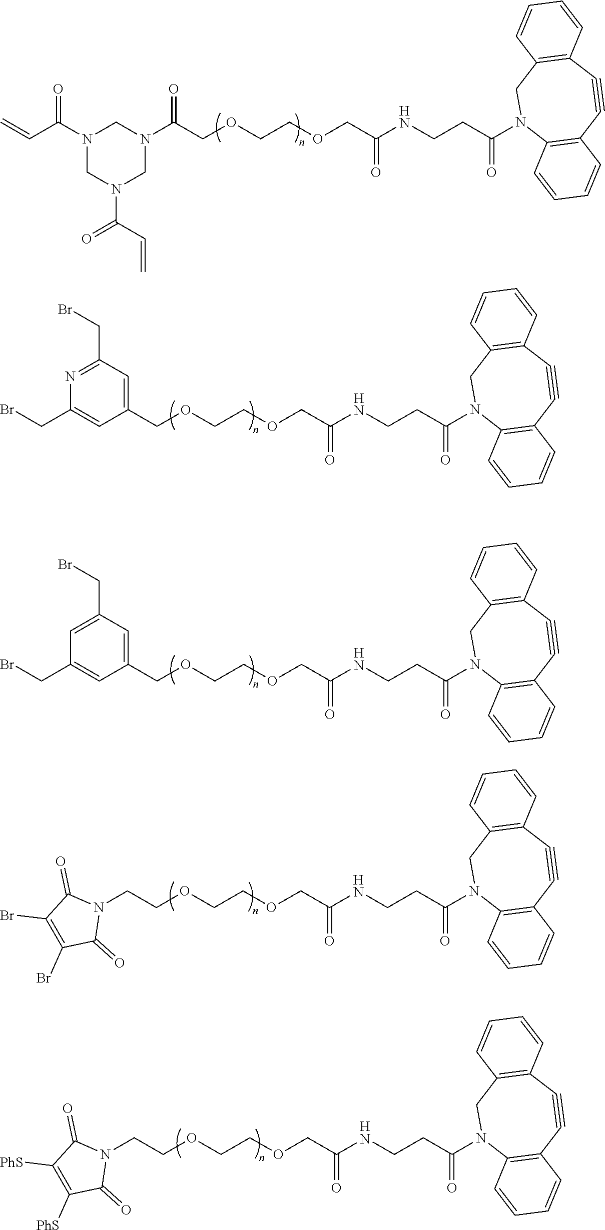

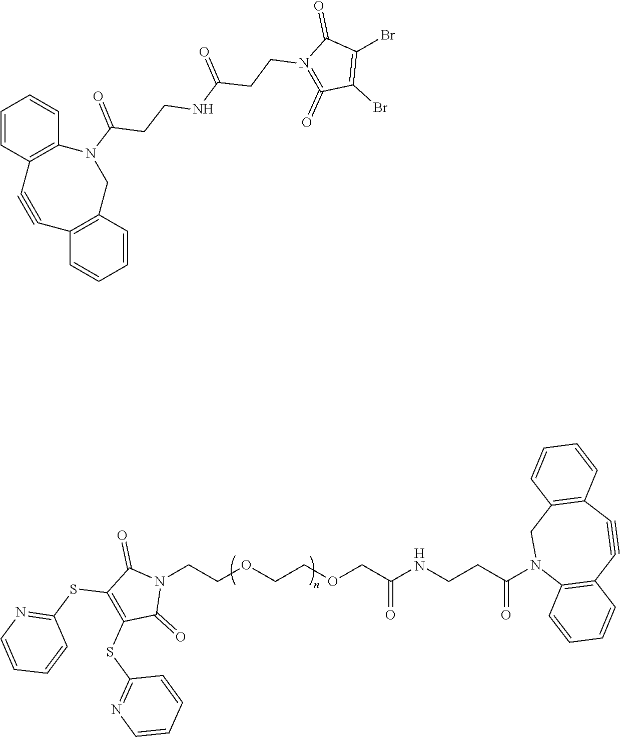

wherein, n is 0-20, 0-18, 0-15, 0-13, 0-10, 0-7, 0-5 or 0-3.

In some embodiments, the first thiobridge reagent bearing reactive groups could be different from the second thiobridge reagent bearing reactive groups. In some embodiments, the first thiobridge reagent bearing reactive groups could be the same as the second thiobridge reagent bearing reactive groups.

In some embodiments, the first thiobridge reagent bearing reactive groups and the second thiobridge reagent bearing reactive groups are dibromomaleimide-PEG4-N3 having the following formula

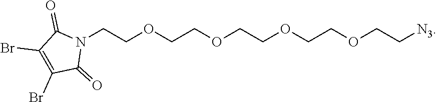 [SMILES: [N-]=[N+]=NCCOCCOCCOCCOCCN1C(=O)C(Br)=C(Br)C1=O]

In some embodiments, a linker of the first linker-payloads and the second linker-payloads is is selected from any one of which the one terminal can be connected to the reduced thiol groups of the antibody or the reactive groups of the thiobridge reagent, and the other terminal can be connected to the payload.

As used herein, the term “linker” refers to a reactive molecule which contains at least two substituted groups, one of which can covalently bond a drug molecule and the other of which can covalently couple to an antibody or the reactive groups of the thiobrige reagent.

In some embodiments, when the first linker-payload and/or the second linker-payload react(s) with the reduced thiol groups, the linker of the first linker-payload and the second linker-payload independently includes a cleavable linker or a noncleavable linker. Cleavable linkers can be chemically labile and enzyme-labile linkers. Due to the high plasma stability and good intracellular cleaving selectivity and efficiency, enzyme-labile linkers are broadly selected as cleavable linker candidates in ADCs. In some embodiments, enzyme-labile linkers comprise the structure: -maleimidocaproyl-(-MC-), -maleimidocaproyl-peptide moiety- (-MC-peptide moiety-), -p-aminobenzyl alcohol-(-PAB-), or -peptide moiety- or -MC-peptide moiety-PAB-.

In some embodiments, the peptide moiety is dipeptides, tripeptides, tetrapeptides or pentapeptides.

In some embodiments, without the limitation, the dipeptides can be valine-alanine (VA), valine-citrulline (VC), alanine-asparagine (AD), alanine-phenylalanine (AF), phenylalanine-lysine (FK), alanine-lysine (AK), alanine-valine (AV), valine-lysine (VK), lysine-lysine (KK), phenylalanine-citrulline (FC), leucine-citrulline (LC), isoleucine-citrulline (IC), tryptophan-citrulline (WC) or phenylalanine-alanine (FA).

In some embodiments, without the limitation, the tripeptides can be alanine-alanine-asparagine (AAD), glycine-valine-citrulline (GVC), glycine-glycine-glycine (GGG), phenylalanine-phenylalanine-lysine (FFK), glutamic acid-valine-citrulline (EVC), or glycine-phenylalanine-lysine (GFK).

In some embodiments, without the limitation, the tetrapeptides can be glycine-glycine-phenylalanine-glycine (GGFG).

In some embodiments, without the limitation, when the first linker-payload and/or the second linker-payload react(s) with the reduced thiol groups, the linker of the first linker-payload and the second linker-payload can be MC-VA-PAB, MC-VC-PAB, MC-AD-PAB, MC-AF-PAB, MC-FK-PAB, MC-AK-PAB, MC-AV-PAB, MC-VK-PAB, MC-KK-PAB, MC-FC-PAB, MC-LC-PAB, MC-IC-PAB, MC-WC-PAB or MC-FA-PAB independently. In some embodiments, without the limitation, when the first linker-payload and/or the second linker-payload react(s) with the reduced thiol groups, the linker of the first linker-payload and the second linker-payload can be MC-AAD-PAB, MC-GVC-PAB, MC-GGG-PAB, MC-FFK-PAB, MC-EVC-PAB, or MC-GFK-PAB independently. In some embodiments, without the limitation, when the first linker-payload and/or the second linker-payload react(s) with the reduced thiol groups, the linker of the first linker-payload and the second linker-payload can be MC-GGFG.

In some embodiments, the linker comprises a maleimide bearing a drug, an organic chloride bearing a drug, an organic bromide bearing a drug, an organic iodide bearing a drug and/or vinylpyrimidine bearing a drug.

In some embodiments, when the first linker-payload and/or the second linker-payload react(s) with the reactive groups in the thiobridge reagent, the linker of the first linker-payload and/or the second linker-payload further include(s) azido and/or dibenzocyclooctyne (DBCO).

In some embodiments, when the linker of the first linker-payload and/or the second linker-payload contains azido, the reactive groups of the thiobridge group contain DBCO. In some embodiments, when the linker of the first linker-payload and/or the second linker-payload contains DBCO, the reactive groups of the thiobridge group contain azido.

In some embodiments, when the linker of the first linker-payload and/or the second linker-payload react(s) with the reactive groups in the thiobridge reagent, the linker of the first linker-payload and the second linker-payload is independently selected from any one of the groups consisting of

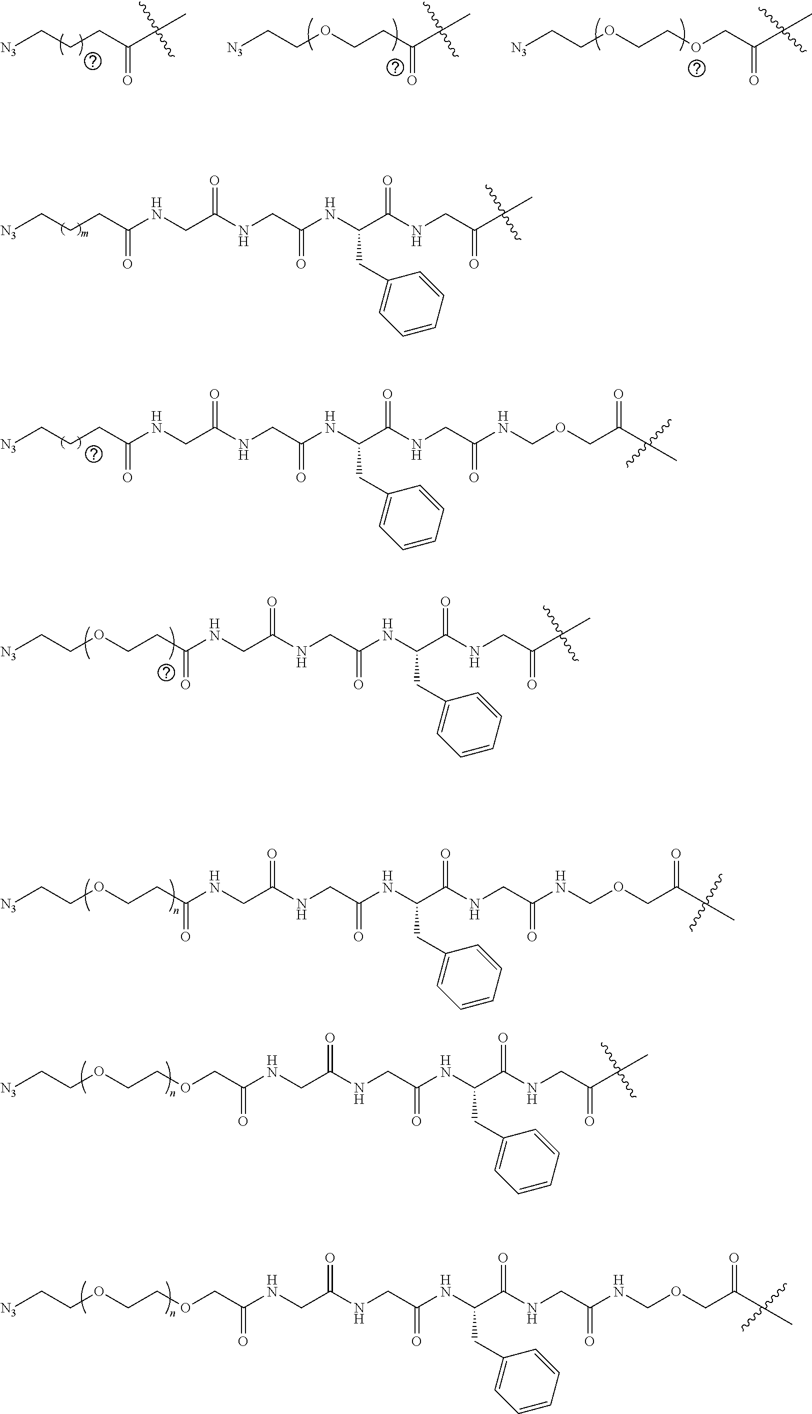

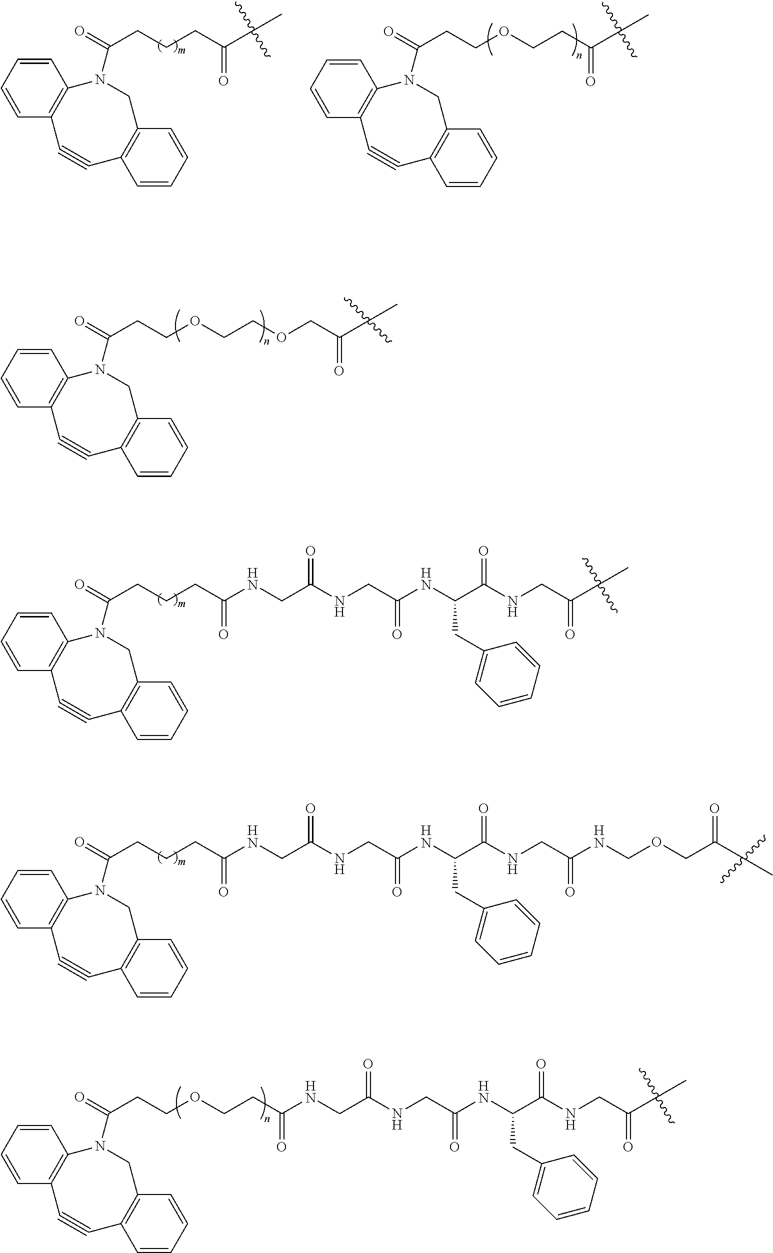

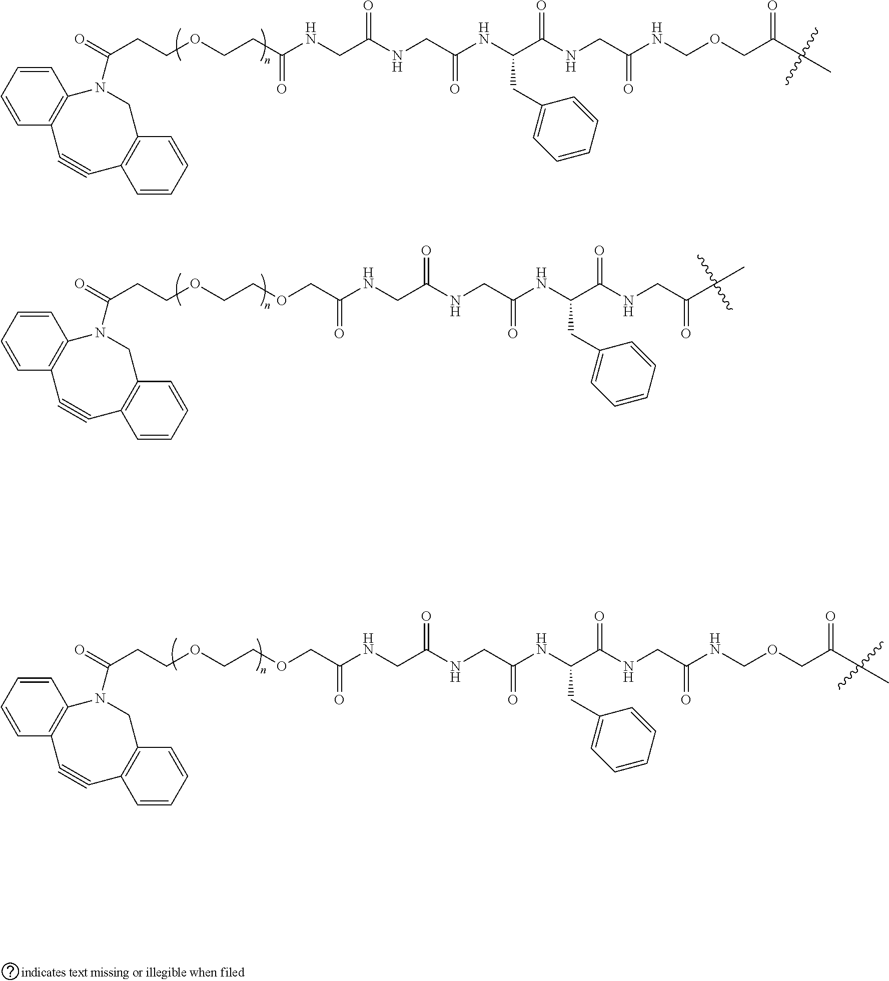

wherein, n is 0-20, 0-18, 0-15, 0-13, 0-10, 0-7, 0-5 or 0-3, m is 0-20, 0-18, 0-15, 0-13, 0-10, 0-7, 0-5 or 0-3, optionally, n is 0, 1, 2, 3, 4, 5, 6, 7, 8, 9 or 10, m is 0, 1, 2, 3, 4, 5, 6, 7, 8, 9 or 10.

As used herein, the term “end capping reagent” refers to a compound which does not bear a drug and contains at least one substituted group which can covalently couple to an antibody.

In some embodiments, the end capping reagent is the cleavable linker or the noncleavable linker. In some embodiments, the end capping reagent is (2-Aminoethyl) maleimide.

In some embodiments, the first thiobridge reagent bearing the first linker-payload and the second thiobridge reagent bearing the second linker-payload have the following formula:

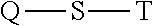

Wherein, Q is selected from the groups consisting of

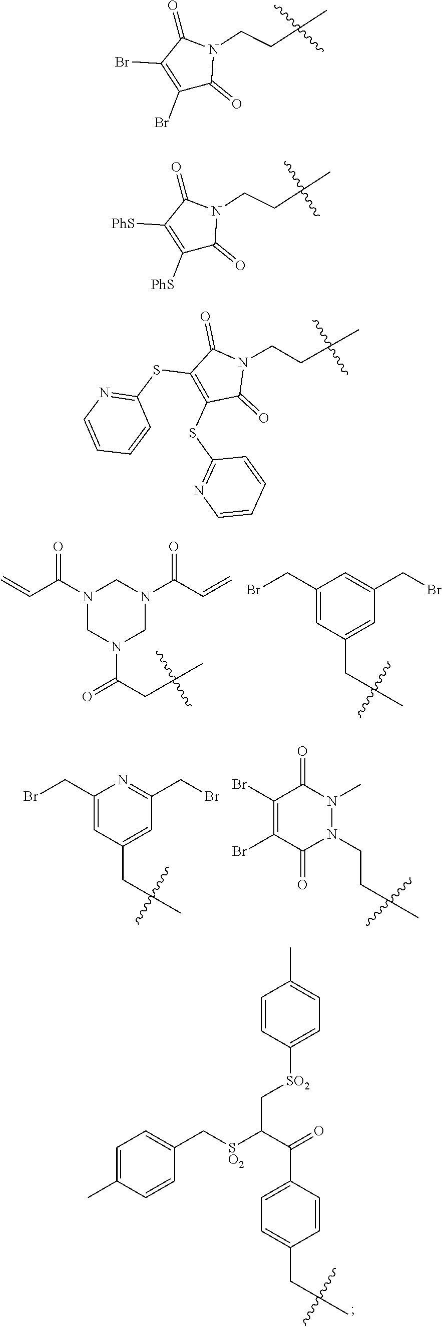

S is selected from a cleavable linker or a non-cleavable linker, without the limitation, S is selected from the groups consisting of

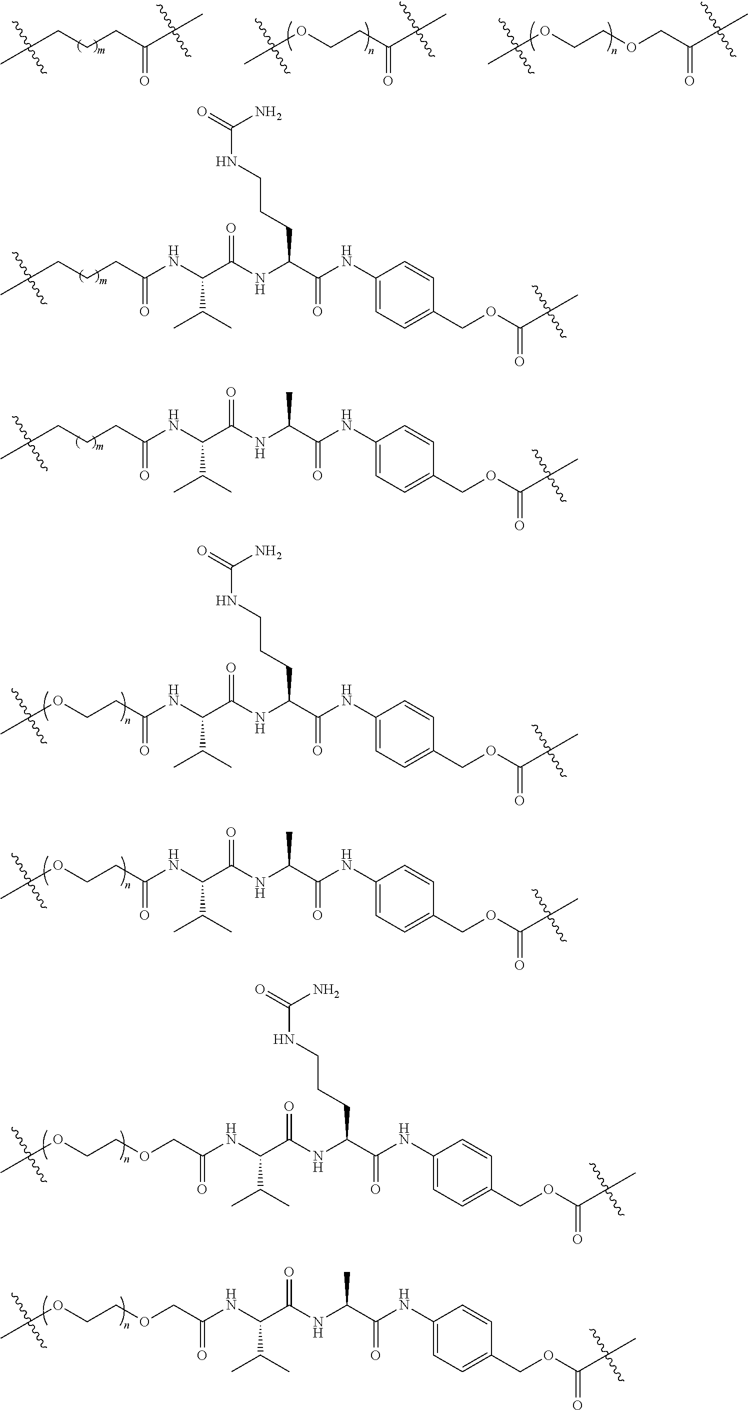

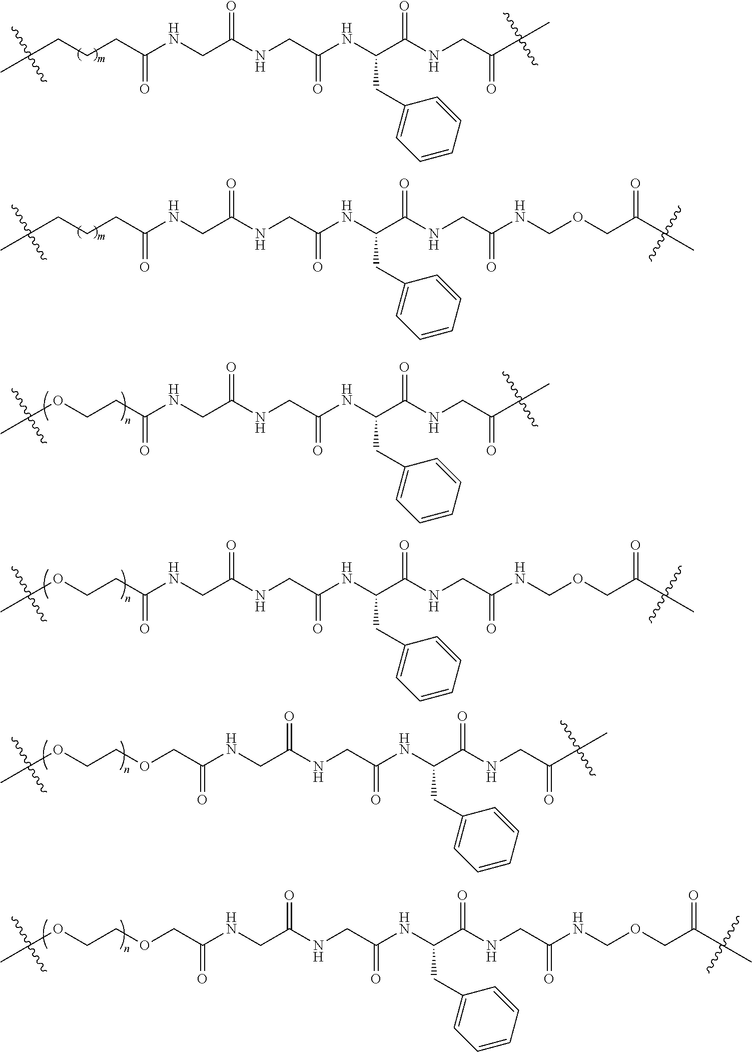

wherein, n is 0-20, m is 0-20, optionally, n is 0, 1, 2, 3, 4, 5, 6, 7, 8, 9 or 10, m is 0, 1, 2, 3, 4, 5, 6, 7, 8, 9 or 10.
T is payload.

In some embodiments, the payload is selected from any one of which contains at least one substituted group allowing a connection from the payload to the linker.

As used herein, the term “payload” refers to any cytotoxic molecule or any molecule of medical interest at least one substituted group or a partial structure allowing a connection from the payload to the linker structure. The payload may kill cancer cells and/or inhibit growth, proliferation, or metastasis of cancer cells, thereby reducing, alleviating, or eliminating one or more symptoms of a disease or disorder.

In some embodiments, the payload is a cytotoxic drug, a fluorescent dye, a cytokine, a nucleic acid, a radionuclide, a kinase inhibitor or derivatives thereof. In some embodiments, the payload includes but not limited to topoisomerases inhibitor and tubulin inhibitors. In some embodiments, without the limitation, the payload can be anti-cancer agent, antiviral agent or antimicrobial agent.

In some embodiments, the cancer is carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Exemplary payloads are monomethyl auristatin E (MMAE), monomethyl auristatin D (MMAD), monomethyl auristatin EF (MMAF), calicheamicins (CLM), mertansine (DM1), maytansinoids, duocarmycins, anthracyclines, pyrrolobenzodiazepine dimers, amatoxin, quinolinealkaloid, Dxd, doxorubicin hydrochloride, methotrexate, erlotinib, bortezomib, fulvestrant, sunitib imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, vincristine, vinblastine, vinorelbine, vindesine, etoposide, teniposide, etoposide phosphate, epipodophyllotoxins, actinomycin, daunorubicin, valrubicin, idarubicin, edrecolomab, epirubicin bleomycin, plicamycin or mitomycin, and salts thereof.

In some embodiments, the payload is deruxtecan (DXd), Cy3 (cyanine 3), MMAE, MMAD or MMAF. In some embodiments of the present application, the payload is MMAE, DXd or Cy3.

The linker-payload is a chemical moiety, which is synthesized by connecting a linker to a payload. Depending on the desired payload and selected linker, those skilled in the art can select suitable method for coupling them together. For example, some conventional coupling methods, such as amine coupling methods, may be used to form the desired linker-payload which still contains reactive groups for conjugating to the antibodies through covalent linkage. A drug-maleimide complex (i.e., maleimide linking drug) is taken as an example of the payload bearing reactive group in the present disclosure. Most common reactive group capable of bonding to thiol group in ADC preparation is maleimide. Additionally, organic chloride, bromides, iodides also are frequently used.

The linker-payload could be any physical active compound, or any compound used to diagnose, prevent or treat a disease.

In some embodiments, when the first linker-payload and/or the second liner-payload react(s) with the reduced thiol groups, the first linker-payload and/or the second linker-payload are independently MC-GGFG-DXd, MC-VC-PAB-MMAE, MC-VC-PAB-MMAD and MC-VC-PAB-MMAF.

In some embodiments, the first thiobridge reagent bearing the first linker-payload and the second thiobridge reagent bearing the second linker-payload, without the limitation, are independently selected from the groups consisting of

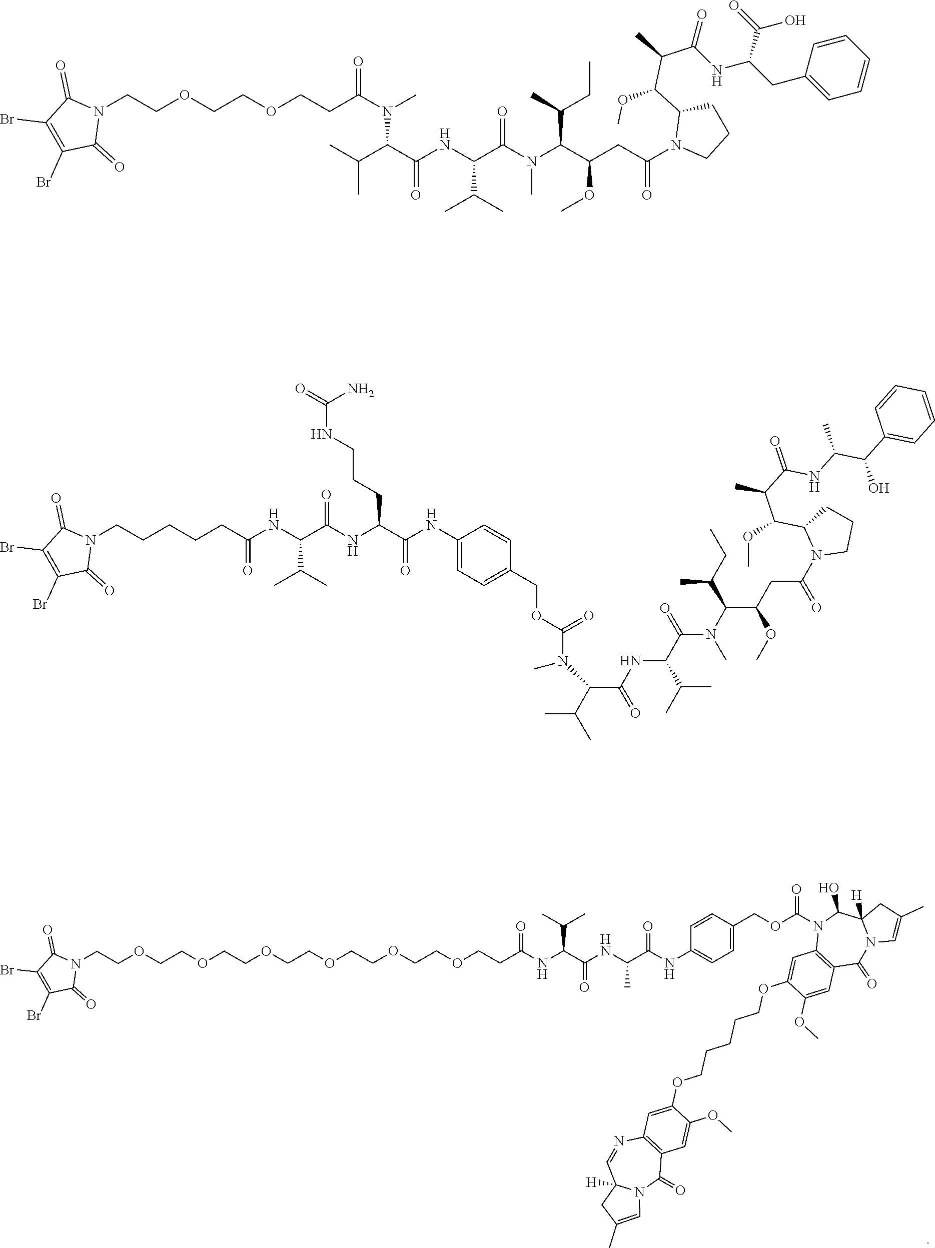

In some embodiments, the payload of the first thiobridge reagent bearing the first linker-payload and that of the second thiobridge reagent bearing the second linker-payload are different. In some embodiments, the linker of the first thiobridge reagent bearing the first linker-payload and that of the second thiobridge reagent bearing the second linker-payload could be different. In some embodiments, the linker of the first thiobridge reagent bearing the first linker-payload and that of the second thiobridge reagent bearing the second linker-payload could be the same. In some embodiments, the thiobridge reagent of the first thiobridge reagent bearing the first linker-payload and that of the second thiobridge reagent bearing the second linker-payload could be different. In some embodiments, the thiobridge reagent of the first thiobridge reagent bearing the first linker-payload and that of the second thiobridge reagent bearing the second linker-payload could be the same.

In some embodiments, there is no specific limitation to the antibody. According to the antigens associated with the disease, those skilled in the art can select suitable antibody useful in the bio-conjugation process of the present application.

In some embodiments, the antibody is a monoclonal antibody, a polyclonal antibody, a mono-specific antibody or a multi-specific antibody.

As used herein, the term “antibody” refers to any immunoglobulin that binds to a specific antigen. A native intact antibody comprises two heavy chains and two light chains. Each heavy chain consists of a variable region and a first, second, and third constant region, while each light chain consists of a variable region and a constant region. The heavy chain from any vertebrate species can be assigned to one of five different classes (or isotypes): IgA, IgD, IgE, IgG, and IgM.

As used herein, the term “Fc region” refers to a monomeric, dimeric or heterodimeric protein having at least an immunoglobulin CH2 and CH3 domain. The CH2 and CH3 domains can form at least a part of the dimeric region of the protein/molecule (e.g., antibody).

In some embodiments, the antibody is a human antibody, a humanized antibody, a chimeric antibody or an antigen-binding moiety thereof.

As used herein, the term “human antibody” refers to one which possesses an amino acid sequence which corresponds to that of an antibody produced by a human or a human cell or derived from anon-human source that utilizes human antibody repertoires or other human antibody-encoding sequences. This definition of a human antibody specifically excludes a humanized antibody comprising non-human antigen-binding residues.

As used herein, the term “humanized antibody” refers to a chimeric antibody comprising amino acid residues from non-human heavy chain variable regions (HVRs) and amino acid residues from human FRs. In certain embodiments, a humanized antibody will comprise substantially all or at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

As used herein, the term “chimeric antibody” refers to an antibody in which a portion of the heavy and/or light chain is derived from a particular source or species, while the remainder of the heavy and/or light chain is derived from a different source or species.

In some embodiments, the antibody means an immunoglobulin and is a molecule containing an antigen-binding site immunospecifically binding to an antigen. In some embodiments of the present application, the class of the antibody is IgG, IgE, IgM, IgD, IgA, or IgY. In some embodiments of the present application, the class of the antibody is IgG.

In some embodiments, the class of the antibody is IgG1, IgG2, IgG3 or IgG4. In some embodiments, the antibody is IgG1 or IgG4.

In some embodiments of the present application, the antibody comprises at least one mutation in the Fc region. In some embodiments, the at least one mutation modulates effector function, or attenuates or eliminates Fc-g receptor binding.

In some embodiments, the one or more mutations are to stabilize the antibody and/or to increase half-life. In some instances, the one or more mutations are to modulate Fe receptor interactions, to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

In some embodiments, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some embodiments, the Fc region comprises a mutation at residue position L234, L235, D265, N21, K46, L52, or P53, or a combination thereof. In some instances, the mutations comprise L234 and L235 in combination with a mutation at residue position K46, L52, or P53.

In some embodiments, the Fc region comprises mutations at L234, L235, and K46. In some cases, the Fc region comprises mutations at L234, L235, and L52. In some cases, the Fc region comprises mutations at L234, L235, and P53. In some cases, the Fe region comprises mutations at D265 and N21. In some cases, the residue position is in reference to IgG1.

In some instances, the Fe region comprises L234A, L235A, D265A, N21G, K46G, L52R, or P53G, or a combination thereof. In some instances, the Fe region comprises L234A and L235A in combination with K46G, L52R, or P53G. In some cases, the Fe region comprises L234A, L235A, and K46G. In some cases, the Fc region comprises L234A, L235A, and L52R.

In some cases, the Fe region comprises L234A, L235A, and P53G. In some cases, the Fe region comprises D265A and N21G. In some cases, the residue position is in reference to IgG1.

In some embodiments, the Fe region comprises a mutation at residue position L233, L234, D264, N20, K45, L51, or P52. In some instances, the Fc region comprises mutations at L233 and L234. In some instances, the Fc region comprises mutations at L233 and L234 in combination with a mutation at residue position K45, L51, or P52. In some cases, the Fe region comprises mutations at L233, L234, and K45. In some cases, the Fc region comprises mutations at L233, L234, and L51. In some cases, the Fc region comprises mutations at L233, L234, and K45. In some cases, the Fe region comprises mutations at L233, L234, and P52. In some instances, the Fe region comprises mutations at D264 and N20. In some cases, equivalent positions to residue L233, L234, D264, N20, K45, L51, or P52 in an IgG1, IgG2, IgG3, or IgG4 framework are contemplated.

In some embodiments, the Fc region comprises L233A, L234A, D264A, N20G, K45G, L51R, or P52G. In some instances, the Fc region comprises L233A and L234A. In some instances, the Fe region comprises L233A and L234A in combination with K45G, L51R, or P52G. In some cases, the Fe region comprises L233A, L234A, and K45G. In some cases, the Fc region comprises L233A, L234A, and L51R. In some cases, the Fe region comprises L233A, L234A, and K45G. In some cases, the Fc region comprises L233A, L234A, and P52G. In some instances, the Fc region comprises D264A and N20G.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al, 2006 PNAS, 103(11): 4005-4010, Shields et al, 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the antibody of IgG1, IgG2, IgG3 or IgG4 is human or humanized antibody. The information of IgG1, IgG2, IgG3 or IgG4 can be obtained on NCBI or UniProt (https://www.uniprot.org/).

In some embodiments, the antibody is bispecific antibodies. In some embodiments of the present application, the antibody is IgG1 like bispecific antibodies.

In some embodiment, those skilled in the art can select suitable method to prepare the bispecific antibodies. In some embodiments of the present application, the bispecific antibodies can be obtained by Knobs-in-holes technology (Ridgway J B B, Presta L G, Paul C. ‘Knobs-into-holes’ engineering of antibody CH3 domains for heavy chain heterodimerization[J]. Protein Engineering(7):617(2023-08-11).), format chain exchange (FORCE) technology, a common light chain format technology (De Nardis C, Hendriks L J A, Poirier E, et al A new approach for generating bispecific antibodies based on a common light chain format and the stable architecture of human immunoglobulin G1[J]. Journal of Biological Chemistry, 2017:jbc.M117.793497.), controlled Fab arm exchange technology (Yanakieva De, Pekar L, Evers A, et al. Beyond bispecificity: Controlled Fab arm exchange for the generation of antibodies with multiple specificities[J]. MABS, 2022, 14(1), e2018960), CrossMAb technology (Klein C, Schaefer W, Regula J T. The use of CrossMAb technology for the generation of bi- and multispecific antibodies[J]. MABS, 2016, 8(6), P1010-P1020.) or their combination.

As used herein, the term “knobs-into-holes” is used in its broadest sense and encompasses various situations, such as the CH1 domain of one heavy chain with the knob mutations and the CH1 domain of the other heavy chain with the hole mutations, the CH2 domain of one heavy chain with the knob mutations and the CH2 domain of the other heavy chain with the hole mutations, and/or the CH3 domain of one heavy chain with the knob mutations and the CH3 domain of the other heavy chain with the hole mutations. For example, and generally, “knobs-into-holes” may refer to an intra-interface modification between two antibody heavy chains in the CH3 domains: i) in the CH3 domain of one heavy chain (first CH3 domain), an amino acid residue is substituted with another amino acid residue bearing a large side chain, thereby creating a protrusion (“knob”) in the interface in the first CH3 domain; ii) in the CH3 domain of the other heavy chain (second CH3 domain), an amino acid residue is substituted with another amino acid residue bearing a smaller side chain, thereby creating a cavity (“hole”) within the interface in the second CH3 domain, in which a protrusion (“knob”) in the first CH3 domain can be placed.

In some embodiments, the antibody is selected from any one of cytotoxic antibodies, inhibitors of cell proliferation, regulators of cell activation and interaction, regulators of the human immune system, neutralizations of antigens, antibodies that are immunospectific for viral antigens or antibodies that are immunospectific for microbial antigens.

In some embodiments of the present application, the antibody can be target-specific antibodies, In some embodiments of the present application, without the limitation, the antibody can be anti-HER2 antibody, anti-FAP antibody, anti-OX-40 antibody, anti-41BB antibody, anti-Angiopoietin-2 antibody, anti-ant-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CD79 antibody, anti-CEA antibody, anti-CGPR/CGRPR antibody, anti-CSPGs antibody, anti-CTLA4 antibody, anti-CTLA-4domains antibody, anti-DLL-4 antibody, anti-EGFR antibody, anti-EpCAM antibody, anti-factor IXa antibody, anti-factor X antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-1R antibody, anti-IGF2 antibody, anti-IGFR antibody, anti-IL-1 antibody, anti-IL-12 antibody, anti-IL-12p40 antibody, anti-IL-1β antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-CoV-2 antibody, anti-Te38 antibody, anti-TIM-3 antibody, anti-TNF antibody, anti-TNFα antibody, anti-TROP-2 antibody, anti-TWEAK antibody, anti-VEGF antibody, or anti-VEGFR antibody.

In some embodiments, the antibody is target-specific, which is targeted to, HER2 (Human Epidermal GrowthFactor Receptor 2), TROP2 (TACSTD2, tumor associated calcium signal transducer 2), BCMA (TNFRSF17, TNF receptor superfamily member 17).

In some embodiments of the present application, the antibody can be Transtuzumab, Sacituzumab, Belantamab, Risankizumab, Eptinezumab, Teprotumumab, Polatuzumab, Tafasitamab, Rovelizumab, Romosozumab, Dostarlimab, Enfortumab or Ublituximab.

In some embodiments, the antibody is Trastuzumab, Sacituzumab or Belantamab.

In some embodiments, the antibody can be obtained commercially or produced by any method known to those skilled in the art.

In some embodiments, the method of preparing the ADC with D6 comprises the following steps,
(1) TCEP (4 eq-15 eq) and ZnCl₂ (the molar ratio of Zn/TCEP is 0.1:1 to 30:1) were added to a solution of Trastuzumab (0.01 mM-0.2 mM) in BES buffer (20 mM, pH7.0) and the reaction mixture was vortexed for mixing, then the reaction mixture was incubated at 4° C. for 4 h-18 h;
(2) EDTA (0.6 mM) and MC-VC-PAB-MMAE (0.08 mM) in DMA was introduced and the reaction was incubated at room temperature for 1 h;
(3) Optionally, introducing cysteine to consume excessive MC-VC-PAB-MMAE;
(4) The reaction mixture was subjected to purification using a de-salting column.

In some embodiments, the method of preparing the bi-payload ADC with D6+D2 comprises the following steps,
(1) ZnCl₂ (the molar ratio of Zn/TCEP is 0.1:1 to 30:1) and the first reductant TCEP (4 eq-15 eq) were added to a solution of a monoclonal antibody Trastuzumab (0.01 mM-0.2 mM) in BES buffer (pH7.0, 20 mM) and the reaction mixture was allowed to stay at 4° C. for 18 h;
(2) EDTA-2Na (0.6 mM) and MC-GGFG-DXd (0.1 mM) in DMA was introduced and the reaction was continued at room temperature for 1 h;
(3) The resulting product and the second reductant TCEP (0.02 mM) were incubating at room temperature for 2 h; a second linker-payload MC-VC-PAB-MMAE (0.05 mM) was added and the reaction was continued at room temperature for 2 h.
(4) purifying the resultant product using a desalting column.

In some embodiments, the method of preparing the bi-payload ADC with D6+D1 comprises the following steps,
(1) ZnCl₂ (the molar ratio of Zn/TCEP is 0.1:1 to 30:1) and the first reductant TCEP (4 eq-15 eq) were added to a solution of a monoclonal antibody Trastuzumab (0.01 mM-0.2 mM) in BES buffer (pH7.0, 20 mM) and the reaction mixture was allowed to stay at 4° C. for 18 h;
(2) EDTA-2Na (0.6 mM) and MC-GGFG-DXd (0.1 mM) in DMA was introduced and the reaction was continued at room temperature for 11 h;
(3) The resulting product and the second reductant TCEP (0.02 mM) were incubating at room temperature for 2 h; a thiobridge reagent with the reactive group Dibromomaleimide-PEG4-N3 (0.012 mM) was added and the reaction was continued at room temperature for 2 h; then a second linker-payload DBCO-Cy3 (0.05 mM) added and the reaction was continued at room temperature for 4 h.
(4) purifying the resultant product using a desalting column.

In some embodiments, the method of preparing the ADC with D3 comprises the following steps,
(1) ZnCl₂ (the molar ratio of Zn/TCEP is 0.1:1 to 30:1) and the first reductant TCEP (4 eq-15 eq) were added to a solution of a monoclonal antibody Trastuzumab (0.01 mM-0.2 mM) in BES buffer (pH7.0, 20 mM) and the reaction mixture was allowed to stay at 4° C. for 18 h;
(2) introducing EDTA (0.6 mM) and Bismaleimide-DBCO (0.045 mM) to react with reduced thiol groups resulted from step (1) at room temperature for 3 h, then recovering the product using a desalting column to afford Trastuzumab-[Bismaleimide-DBCO]₃;
(3) incubating Trastuzumab-[Bismaleimide-DBCO]₃ and N3-Cy3 (0.06 mM) in BES buffer (20 mM, pH6.7), the reaction temperature is 25° C. and the reaction time is 6 h, then recovering Trastuzumab-[Bismaleimide-DBCO-N3-Cy3]₃ using a desalting column.

In some embodiments, the method of preparing the ADC with D0+D2 comprises the following steps,
(1) ZnCl₂ (the molar ratio of Zn/TCEP is 0.1:1 to 30:1) and the first reductant TCEP (4 eq-15 eq) were added to a solution of a monoclonal antibody Trastuzumab (0.01 mM-0.2 mM) in BES buffer (pH7.0, 20 mM) and the reaction mixture was allowed to stay at 4° C. for 18 h;
(2) introducing EDTA (0.6 mM) and (2-Aminoethyl) maleimide (0.04 mM) to react with reduced thiol groups resulted from step (1) at room temperature for 3 h, then recovering the product using a desalting column to afford Trastuzumab-Maleimide;
(3) Trastuzumab-Maleimide and reductant TCEP (0.02 mM) were incubating at room temperature for 2 h; a second linker-payload MC-VC-PAB-MMAE (0.05 mM) was added and the reaction was continued at room temperature for 2 h.
(4) purifying the resultant product using a desalting column.

In some embodiments, the method of preparing the ADC with D0+D1 comprises the following steps,
(1) ZnCl₂ (the molar ratio of Zn/TCEP is 0.1:1 to 30:1) and the first reductant TCEP (4 eq-15 eq) were added to a solution of a monoclonal antibody Trastuzumab (0.01 mM-0.2 mM) in BES buffer (pH7.0, 20 mM) and the reaction mixture was allowed to stay at 4° C. for 18 h;
(2) introducing EDTA (0.6 mM) and (2-Aminoethyl) maleimide (0.04 mM) to react with reduced thiol groups resulted from step (1) at room temperature for 3 h, then recovering the product using a desalting column to afford Trastuzumab-Maleimide;
(3) Trastuzumab-Maleimide and the second reductant TCEP (0.02 mM) were incubating at room temperature for 2 h. a second thiobridge reagent with the reactive group dibromomaleimide-PEG4-N3 (0.013 mM) to react with the reduced thiol groups, the reaction temperature is 24° C. and the reaction time is 3 h;
(4) incubating resulting product and DBCO-Cy3 (0.02 mM) in MES (20 mM, pH6.7), the reaction temperature is 25° C. and the reaction time is 8 h, then recovering Trastuzumab-[Maleimide]₆[Maleimide-PEG4-N3-DBCO-Cy3]₁ using a desalting column.

Various analytical methods can be used to determine the yields and isomeric mixtures of the ADC. In some embodiments of the present application, the analytical method is HIC-HPLC. HIC-HPLC is able to separate the ADC which antibodies loaded with various numbers of drugs. The drug loading level can be determined based on the ratio of absorbances, e.g., at 250 nm and 280 nm. For example, if a drug can absorb at 250 nm while the antibody absorbs at 280 nm. The 250/280 ratio therefore increases with drug loading. Using the bio-conjugation process described herein, generally antibodies with even numbers of drugs were observed to be conjugated to the antibody since reduction of disulfides yields even numbers of free cysteine thiols. As compared with ADCs generated by conventional conjugation processes, the ADCs of the present application have improved homogeneity.

The Antibody with Site-Specific Modification

On the sixth aspect, the present application provides an antibody with site-specific modification prepared by the method of the present application.

In some embodiments, the sequence of the antibody with site-specific modification is wild type.

As use herein, the term “the antibody of wild type” refers to naturally occurring antibodies without mutation.

In some embodiments, the glycan of the antibody with site-specific modification is not modified.

In some embodiments, the antibody with site-specific modification dose not refer to antibody engineering, enzyme technologies and glycan modification.

In some embodiments, the antibody with site-specific modifications is conjugated with the modification reagent 1, forming the ADC with D6, the ADC with D1, the ADC with D3, the ADC with D2, the ADC with D4 or the ADC with D6. In some embodiments, the antibody with site-specific modifications is conjugated with the modification reagent 1 and the modification reagent 2, forming the bi-payload ADC with D6+D2, the bi-payload ADC with D6+D1, the bi-payload ADC with D3+D1, the bi-payload ADC with D3+D2, the ADC with D0+D2, the ADC with D0+D1, the bi-payload ADC with D2+D6, the bi-payload ADC with D2+D3, the bi-payload ADC with D1+D6, the bi-payload ADC with D1+D3, the ADC with D0+D6, the ADC with D0+D3, the bi-payload ADC with D2+D4, the bi-payload ADC with D2+D2, the bi-payload ADC with D1+D4 or the bi-payload ADC with D1+D2.

In some embodiments, the antibody with site-specific modifications is the ADC with D2, the ADC with D4, the ADC with D1, the ADC with D6, the ADC with D3, the ADC with D1+D6, the ADC with D6+D2, the ADC with D6+D1, the ADC with D3+D1, the ADC with D3+D2, the ADC with D0+D6, the ADC with D0+D2, the ADC with D1+D2, the ADC with D1+D4 or the ADC with D2+D4.

In some embodiments, the antibody with site-specific modifications is Trastuzumab-[MC-VC-PAB-MMAE]₆, Sacituzumab-[MC-VC-PAB-MMAE]₆, Belantamab-[MC-VC-PAB-MMAE]₆, Trastuzumab-[MC-VC-PAB-MMAE]₂, Trastuzumab-[Bismaleimide-DBCO]₃, Trastuzumab-[MC-GGFG-DXd]₆[MC-VC-PAB-MMAE]₂, Trastuzumab-[MC-VC-PAB-MMAE]₆[Maleimide-PEG4-N3-DBCO-Cy3]ᵢ, Trastuzumab-[Maleimide]₆[MC-VC-PAB-MMAE]₂, Trastuzumab-[Maleimide]₆[Maleimide-PEG4-N3-DBCO-Cy3]ᵢ, Trastuzumab-[Maleimide-PEG4-N3-DBCO-MMAE]ᵢ, Trastuzumab-[Maleimide-PEG4-N3-DBCO-MMAE]₁[MC-GGFG-DXd]₆ or Trastuzumab-[MC-VC-PAB-MMAE]₂[MC-GGFG-DXd]₄.

A pharmaceutical composition comprising the antibody with site-specific modification

On the seventh aspect, the present application provides a pharmaceutical composition comprising the antibody with site-specific modification according to the present application and one or more of pharmaceutically acceptable carrier.

Pharmaceutical compositions provided herein may be formulated in any manner known in the art, such as, pharmaceutical compositions provided herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine.

In some embodiments, the pharmaceutical compositions can be a liquid solution, suspension, or emulsion. In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments, the anti-cancer agents can include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, epirubicin, bleomycin, plicamycin, mitomycin and the like.

In some embodiments, the anti-autoimmune disease agent can include, but not limited to, ibuprofen, loxoprofen, naproxen, diclofenac, indomethacin, meloxicam, lornoxicam, nabumetone, celecoxib, paracetamol, glucocorticoids, azathioprine, cyclophosphamide and the like.

In some embodiments, patients may experience nausea during and after administration of the ADCs of the present application. Therefore, anti-emetics may be administered in preventing nausea (upper stomach) and vomiting. The anti-emetics can include, but not limited to, aprepitant, ondansetron, granisetron HCl, lorazepam, dexamethasone, prochlorperazine, casopitant and the like.

In some embodiments, patients may experience allergic reactions during and after administration of the ADCs of the present application. Therefore, anti-allergic agents may be administered to minimize the risk of an allergic reaction. The anti-allergic agents include dexamethasone, beclomethasone, hydrocortisone, prednisolone, prednisone, methylprednisolone, hydroxyzine, cyproheptadine, bronchodilators, terbutaline and the like.

### The Use of TCEP or Salt Thereof

On the eighth aspect, the present application provides use of TCEP or salt thereof in the preparation of the antibody with site-specific modification.

In some embodiments, the antibody with site-specific modification is modified by selectively reducing the interchain S—S bonds. In some embodiments, the antibody with site-specific modification is modified by selectively reducing three interchain S—S bonds.

In some embodiments, TCEP or salt thereof and the transition metal ions are used together.

In some embodiments, TCEP and the transition metal ions together selectively reduce three of four inter-chain disulfide bonds of antibody with the specific molar ratio of TCEP and antibody, the specific incubation time of TCEP, and introducing the metal chelators after the incubation reaction in step (a).

The Use of the Antibody with Site-Specific Modification

On the ninth aspect, the present application provides use of the antibody with site-specific modification according to the present application in the manufacture of a therapeutic agent for diagnosing, preventing or treating disease.

On the tenth aspect, the present application provides a method of preventing or treating a disease in a subject in need thereof, comprising administrating to the subject a therapeutically effective amount of the antibody with site-specific modification according to the present application.

As use herein, the term “treat” of any disease refers to alleviating or ameliorating the disease (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent” of any disease refers to the prophylactic treatment of the disease; or delaying the onset or progression of the disease.

As use herein, the term “subject” refers to mammals, primates (e.g., humans, male or female), dogs, rabbits, guinea pigs, pigs, rats and mice. In certain embodiments, the subject is a primate. In yet other embodiments, the subject is a human.

As used herein, the term “a therapeutically effective amount” refers to an amount of the antibody with site-specific modification of the present application that will elicit the biological or medical response of a subject, for example, ameliorate symptoms, alleviate conditions, slow or delay disease progression, or prevent a disease, etc. The therapeutically effective amount will vary with the type and severity of the condition to be alleviated. It is to be further understood that for any particular subject, specific dosage regimens should be adjusted over time according to the individual need and the professional judgment of the person administering or supervising the administration of the compositions. In some embodiments of the present application, the therapeutically effective amount is based on a variety of factors, such as the type of disease, the age, weight, sex, medical condition of the patient, the severity, of the condition, the route of administration, and the particular antibody employed. In some embodiments of the present application, the therapeutically effective amount can vary widely, but can be determined routinely using standard methods. In some embodiments of the present application, the therapeutically effective amount can be adjusted based on the pharmacokinetic or pharmacodynamic parameters, which may include clinical effects such as toxic effects and/or laboratory values.

In some embodiments, the disease is a tumor or cancer. In some embodiments, the disease is an autoimmune disease and the like.

In some embodiments, the cancer can include, but not limited to, carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

It will be readily apparent to those skilled in the art that other suitable modifications and adaptations of the present application described herein are obvious and may be made using suitable equivalents without departing from the scope of the disclosure or the embodiments disclosed herein. Having now described the disclosure in detail, the same will be more clearly understood by reference to the following examples, which are included for purposes of illustration only and are not intended to be limiting. Further, unless specifically described otherwise, the reagent and the solvent described in the description can be easily obtained from a commercial supplier.

### EXAMPLES

The invention is further described in the following examples, which do not limit the scope of the invention described in the claims.

### Reagent and Manufacturer

Trastuzumab is commercially available from Roche.

Sacituzumab and Belantamab are commercially available from MedChemExpress.

MC-VC-PAB-MMAE are commercially available from Levena biopharma.

DMA (Dimethylacetamide) is commercially available from Aldrich Sigma.

TCEP is commercially available from Bidepharm.

EDTA is commercially available from Aladdin.

DHAA is commercially available from Aladdin.

(2-Aminoethyl) maleimide is commercially available from Bidepharma.

The buffers are commercially available from Macklin.

### Homogeneity Assays:

The ADCs distribution were analyzed using HIC-HPLC (Agilent1200) with a TSK gel Butyl-NPR column (2.5 μm, 4.6 mm*35 mm) (commercially available from Tosoh Biosciences) at a flow rate of 0.5 mL/min at 25° C. Solvent A was 50 mM K₂HPO₄-3H₂O and 1.5M (NH₄)₂SO₄. Solvent B was 75% v/v 21.3 mM KH₂PO₄, 28.6 mM K₂HPO₄ and 25% v/v isopropanol. The sampler temperature is 4° C. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

### Example 1: Preparation of Trastuzumab-[MC-VC-PAB-MMAE]₆ Conjugate by Using the Process of the Present Application

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) EDTA (0.6 mM) and MC-VC-PAB-MMAE (0.08 mM) in DMA was introduced and the reaction was incubated at room temperature for 1 h;
(3) The reaction mixture was subjected to purification using a de-salting column (Thermo, type: 40K, 0.5 mL, REF:87766, Lot SJ251704,).

### Example 2: Preparation of Sacituzumab-[MC-VC-PAB-MMAE]₆ Conjugate by Using the Process of the Present Application

The method of example 2 is similar to example 1, and the difference is that the antibody is Sacituzumab, the buffer system is MES buffer and the molar ratio of Zn²⁺/TCEP is 0.87:1.

### Example 3: Preparation of Belantamab-[MC-VC-PAB-MMAE]₆ Conjugate by Using the Process of the Present Application

The method of example 3 is similar to example 1, and the difference is that the antibody is Belantamab, the buffer system is MES buffer and the molar ratio of Zn²⁺/TCEP is 0.87:1.

The homogeneity assays results were shown as follows:

TABLE 1
the results of homogeneity assays of examples 1-3

| Example | | | D0 | D2 | D4 | D6 | D8 |
|---|---|---|---|---|---|---|---|
| No. | FIG. | Antibody | (%) | (%) | (%) | (%) | (%) |
| 1 | 1 | trastuzumab | 0 | 0 | 7 | 90 | 3 |
| 2 | 2 | sacituzumab | 0 | 1 | 3 | 85 | 11 |
| 3 | 3 | belantamab | 0 | 1 | 7 | 82 | 10 |

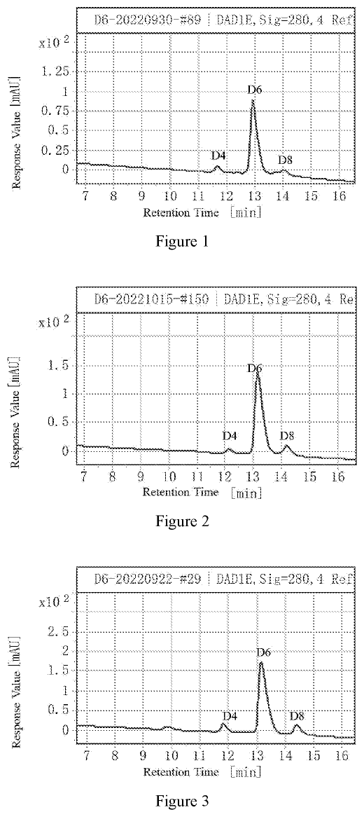

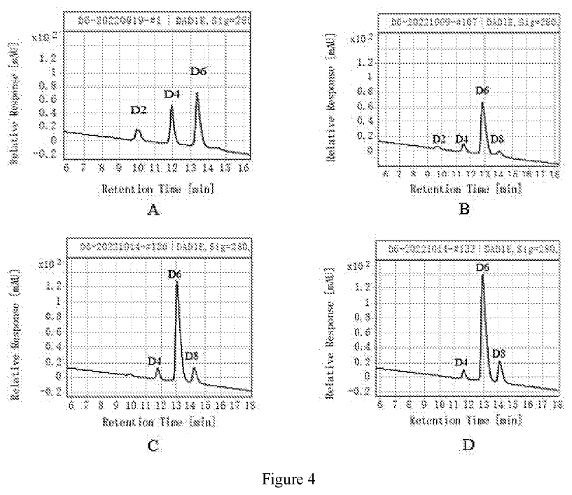

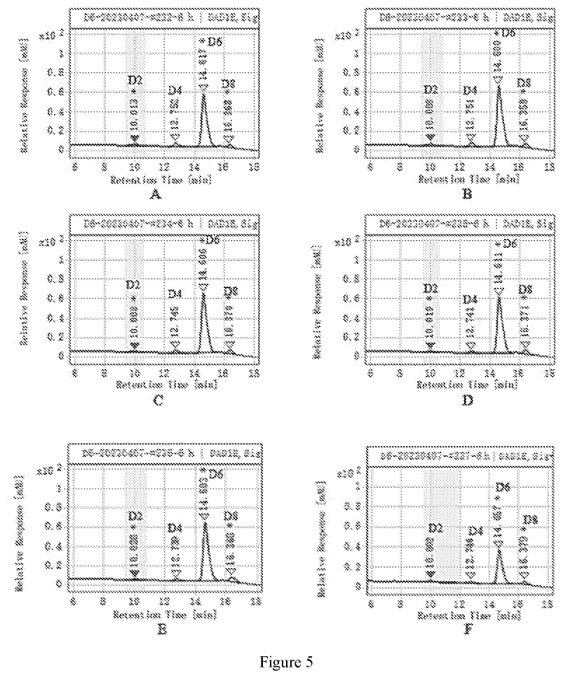

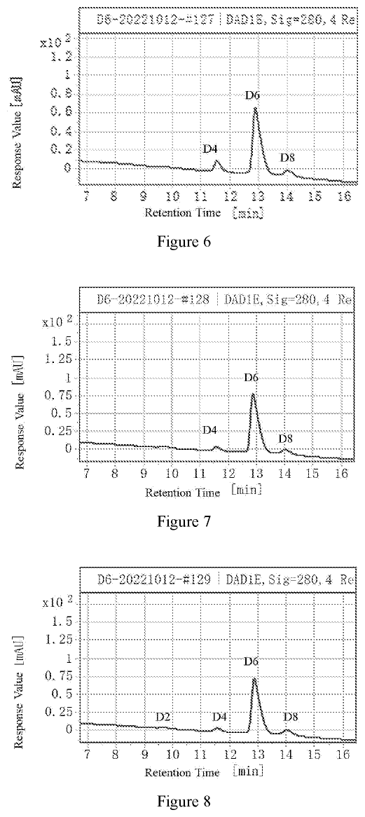

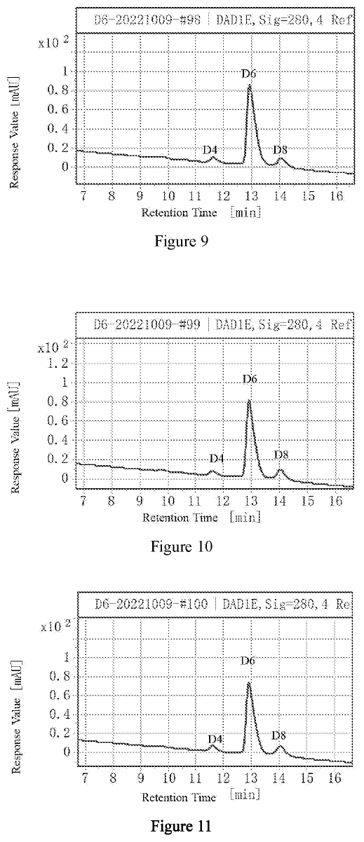

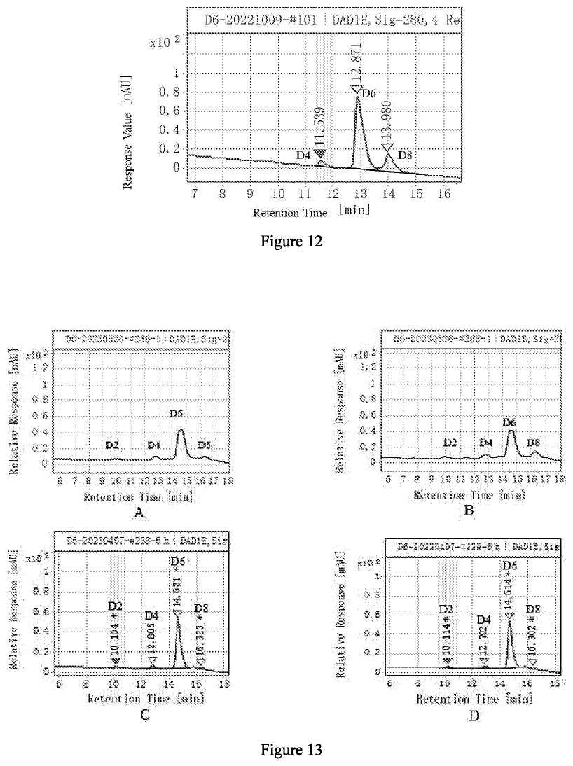

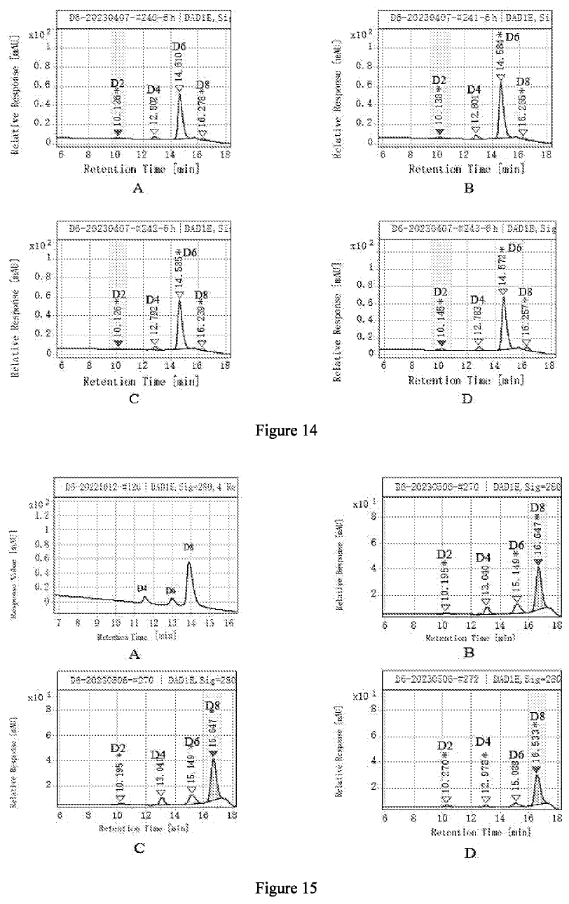

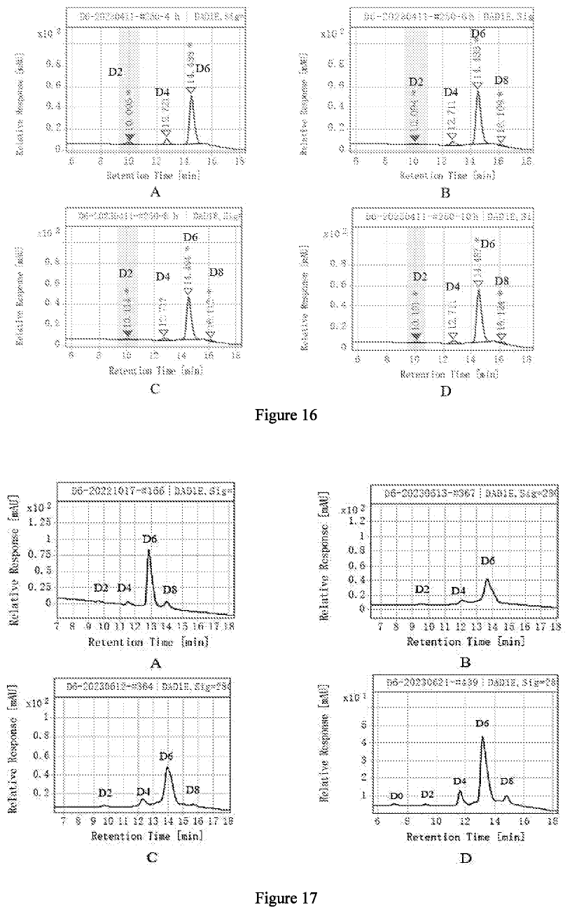

As shown in table 1 and FIGS. 1-3, the results demonstrate MC-VC-PAB-MMAE is successfully linked to Trastuzumab, Sacituzumab and Belantamab, and that the content of D6 in examples 1-3 is generally up to 80%, such as 90%, 85% and 82%. These results clearly indicate that the ADC with D6 prepared with the method of the present application has a significantly improved homogeneity.

### Examples 4-13: Preparation of Antibody-[MC-VC-PAB-MMAE]₆ Conjugate (the Molar Ratio of TCEP and the Antibody and/or the Reduction Time in Step (1) is (are) Different)

The method of examples 4-13 is the same as example 1, and the difference is the concentration of ZnCl₂ and TCEP in step (1) and/or the reduction time in step (1). In example 5 and 6, the antibody is belantamab. The concentration of ZnCl₂ and TCEP and the reduction time in step (1) are as follows:

| | TCEP | TCEP/antibody | Zn²⁺ | Zn²⁺/TCEP | the reduction time |
|---|---|---|---|---|---|
| No. | (mM) | (Molar ratio) | (mM) | (Molar ratio) | in step (1) (h) |
| E4 | 0.036 | 3:1 | 0.048 | 1.33:1 | 18 |
| E5 | 0.0384 | 3.2:1 | 0.144 | 3.75:1 | 18 |
| E6 | 0.06 | 5:1 | 0.048 | 0.80:1 | 18 |
| E7 | 0.072 | 6:1 | 0.048 | 0.67:1 | 18 |
| E8 | 0.096 | 8:1 | 0.024 | 0.25:1 | 6 |
| E9 | 0.108 | 9:1 | 0.024 | 0.22:1 | 6 |
| E10 | 0.12 | 10:1 | 0.024 | 0.20:1 | 6 |
| E11 | 0.132 | 11:1 | 0.024 | 0.18:1 | 6 |
| E12 | 0.144 | 12:1 | 0.024 | 0.17:1 | 6 |
| E13 | 0.156 | 13:1 | 0.024 | 0.15:1 | 6 |

“E” was short for Example.

### Comparative Example 11: Preparation of Antibody-[MC-VC-PAB-MMAE]₂ Conjugate (the ADC with D2)

(1) ZnCl₂ (0.24 mM) and TCEP (0.02 mM) were added to a solution of Transtuzumab (0.012 mM, in MES buffer, pH6.7, 20 mM) and the reaction mixture was allowed to stay at 4° C. for 4 h;
(2) EDTA (0.6 mM) was added to trap Zn²⁺;
(3) MC-VC-PAB-MMAE (0.06 mM) in DMA was introduced and the reaction was continued at 24° C. for 30 min;
(4) cysteine (commercially available from Aladdin, 0.08 mM) was added to deplete excessive MC-VC-PAB-MMAE;
(5) The reaction mixture was subjected to purification using a de-salting column.

The homogeneity assays results of examples E4-E13 and comparative example 11 were shown as follows:

TABLE 2
the results of homogeneity assays of examples 4-13 and the comparative example 11

| | | TCEP/antibody | Reduction time | | | | | |
|---|---|---|---|---|---|---|---|---|
| No. | FIG. | (Molar ratio) | in step (1) (h) | D0(%) | D2(%) | D4(%) | D6(%) | D8(%) |
| C11 | 71 | 1.67:1 | 4 h | 5.22 | 80.6 | 14.17 | 0 | 0 |
| E4 | 4A | 3:1 | 18 | 0 | 12.75 | 32.22 | 55.03 | 0 |
| E5 | 4B | 3.2:1 | 18 | 0 | 2.87 | 11.09 | 80.33 | 5.71 |
| E6 | 4C | 5:1 | 18 | 0 | 0 | 6.27 | 82.40 | 11.32 |
| E7 | 4D | 6:1 | 18 | 0 | 0 | 4.67 | 80.91 | 14.42 |
| E8 | 5A | 8:1 | 6 | 0 | 3.50 | 5.64 | 87.41 | 3.45 |
| E9 | 5B | 9:1 | 6 | 0 | 3.47 | 5.11 | 87.20 | 4.22 |
| E10 | 5C | 10:1 | 6 | 0 | 1.72 | 4.09 | 89.34 | 4.86 |
| E11 | 5D | 11:1 | 6 | 0 | 1.57 | 3.27 | 89.24 | 5.93 |
| E12 | 5E | 12:1 | 6 | 0 | 1.54 | 2.99 | 89.07 | 6.41 |
| E13 | 5F | 13:1 | 6 | 0 | 4.43 | 4.96 | 83.50 | 7.12 |

“C” was short for Comparative Example.

As shown in table 2, compared with comparative example 11, the content of D6 is up to 5500, 80%, even to 85%, and 87% when the molar ratio of TCEP and the antibody is from 3:1 to 13:1. When the molar ratio of TCEP and the antibody is from 8:1 to 13:1, the reduction time in step (1) is shortened to 6 h. When the molar ratio of TCEP and the antibody is from 8:1 to 12:1 and the reduction time in step (1) is 6 h, the content of D6 is up to 87% or 89%. The results showed the molar ratio of TCEP/antibody plays an important role in determining the content of D6 and the selective reduction.

### Examples 14-28: Preparation of Trastuzumab-[MC-VC-PAB-MMAE]₆ Conjugate (the Molar Ratio of Zn²⁺ and TCEP is Different and/or the Reduction Time in Step (1) is (are) Different)

The method of examples 14-28 is similar to example 1, and the difference is the concentration of ZnCl₂ and/or TCEP and/or the reduction time in step (1) which are shown in table 3.

TABLE 3
the concentration of Zn²⁺ of examples 14-28

| | Zn²⁺ | Zn²⁺/TCEP | TCEP | TCEP/antibody | the reduction time |
|---|---|---|---|---|---|
| No. | (mM) | (Molar ratio) | (mM) | (Molar ratio) | in step (1) (h) |
| E14 | 0.012 | 0.25:1 | 0.048 | 4:1 | 18 |
| E15 | 0.024 | 0.5:1 | 0.048 | 4:1 | 18 |
| E16 | 0.048 | 1:1 | 0.048 | 4:1 | 18 |
| E17 | 0.096 | 2:1 | 0.048 | 4:1 | 18 |
| E18 | 0.144 | 3:1 | 0.048 | 4:1 | 18 |
| E19 | 0.192 | 4:1 | 0.048 | 4:1 | 18 |
| E20 | 0.36 | 7.5:1 | 0.048 | 4:1 | 18 |
| E21 | 0.72 | 12:1 | 0.06 | 5:1 | 18 |
| E22 | 1.636 | 27.27:1 | 0.06 | 5:1 | 18 |
| E23 | 0.012 | 0.11:1 | 0.108 | 9:1 | 6 |
| E24 | 0.024 | 0.22:1 | 0.108 | 9:1 | 6 |
| E25 | 0.048 | 0.44:1 | 0.108 | 9:1 | 6 |
| E26 | 0.071 | 0.66:1 | 0.108 | 9:1 | 6 |
| E27 | 0.095 | 0.88:1 | 0.108 | 9:1 | 6 |
| E28 | 0.18 | 1.67:1 | 0.108 | 9:1 | 6 |

### Comparative Examples 1-4: Preparation of Trastuzumab-[MC-VC-PAB-MMAE]₆ Conjugate (the Molar Ratio of and Zn²⁺ and TCEP is Different)

The method of comparative example 1 is similar to example 1, comparative example 2 is similar to example 8, comparative example 3 is similar to example 10, comparative example 4 is similar to example 12, the difference is that the concentration of ZnCl₂ in step (1) is 0.

The homogeneity assays results were shown as follows:

TABLE 4
the results of homogeneity assays of examples 14-28 and comparative examples 1-4

| | | Zn²⁺/TCEP | TCEP/antibody | | | | | |
|---|---|---|---|---|---|---|---|---|
| No. | FIG. | (Molar ratio) | (Molar ratio) | D0(%) | D2(%) | D4(%) | D6(%) | D8(%) |
| C1 | 15A | 0:1 | 4:1 | 0 | 0 | 9 | 10 | 81 |
| C2 | 15B | 0:1 | 8:1 | 0 | 2.64 | 10.01 | 17.10 | 70.25 |
| C3 | 15C | 0:1 | 10:1 | 0 | 1.62 | 6.89 | 11.75 | 79.75 |
| C4 | 15D | 0:1 | 12:1 | 0 | 3.83 | 4.70 | 10.45 | 81.02 |
| E14 | 6 | 0.25:1 | 4:1 | 0 | 0 | 12 | 81 | 7 |
| E15 | 7 | 0.5:1 | 4:1 | 0 | 2 | 5 | 86 | 7 |
| E16 | 8 | 1:1 | 4:1 | 0 | 2 | 5 | 85 | 8 |
| E17 | 9 | 2:1 | 4:1 | 0 | 0 | 4.40 | 86.59 | 9.01 |
| E18 | 10 | 3:1 | 4:1 | 0 | 0 | 4 | 83 | 13 |
| E19 | 11 | 4:1 | 4:1 | 0 | 0 | 6 | 81 | 13 |
| E20 | 12 | 7.5:1 | 4:1 | 0 | 0 | 5 | 75 | 20 |
| E21 | 13A | 12:1 | 5:1 | 0 | 1.88 | 7.48 | 85.81 | 4.83 |
| E22 | 13B | 27.27:1 | 5:1 | 0 | 2.99 | 7.75 | 77.99 | 11.27 |
| E23 | 13C | 0.11:1 | 9:1 | 0 | 2.09 | 5.40 | 90.48 | 2.03 |
| E24 | 13D | 0.22:1 | 9:1 | 0 | 2.57 | 4.79 | 89.99 | 2.65 |
| E25 | 14A | 0.44:1 | 9:1 | 0 | 2.58 | 5.83 | 89.20 | 2.39 |
| E26 | 14B | 0.66:1 | 9:1 | 0 | 2.86 | 6.09 | 89.11 | 1.95 |
| E27 | 14C | 0.88:1 | 9:1 | 0 | 2.55 | 5.82 | 89.59 | 2.03 |
| E28 | 14D | 1.67:1 | 9:1 | 0 | 3.23 | 6.04 | 87.19 | 3.54 |

Comparative Example is abbreviated as “C”.

As shown in table 4, the results showed the content of D6 is up to 75%, even to 80%, 85% and 90% as the molar ratio of Zn²⁺ and TCEP increasing from 0.11:1 to 27.27:1. When the concentration of Zn²⁺ is 0, the content of D6 is as low as 10%, which indicates that the metal transition ions is very important for improving reduction selectively and homogeneity of conjugate with D6.

### Examples 29-32: Preparation of Trastuzumab-[MC-VC-PAB-MMAE]₆ Conjugate (the Reduction Time in Step (1) is Different)

The method of examples 29-32 is similar to example 1, and the difference is the reduction time in step (1) which is shown in the table 5. Meanwhile, the molar ratio of and Zn²⁺ and TCEP is 0.22:1 and the molar ratio of and TCEP and Trastuzumab is 9:1 in examples 29-32.

The homogeneity assays results were shown as follows:

TABLE 5
The results of homogeneity assays of examples 29-32

| | | The reduction | | | | | |
|---|---|---|---|---|---|---|---|
| | | time in | D0 | D2 | D4 | D6 | D8 |
| No. | FIG. | step (1) (h) | (%) | (%) | (%) | (%) | (%) |
| E29 | 16A | 4 | 0 | 3.83 | 9.73 | 86.44 | 0 |
| E30 | 16B | 6 | 0 | 2.39 | 5.87 | 90.62 | 1.13 |
| E31 | 16C | 8 | 0 | 2.09 | 4.66 | 91.65 | 1.60 |
| E32 | 16D | 10 | 0 | 1.88 | 4.00 | 91.34 | 2.79 |

As shown in table 5, the results showed the content of D6 is up to 86%, even to 90% when the molar ratio of TCEP and the antibody is 9:1 and the reductant time in step (1) is from 4 h to 10 h, which indicates that increasing the molar ratio of TCEP and the antibody, the method displayed here is with less reduction time cost.

### Examples 33-36: Preparation of Trastuzumab-[MC-VC-PAB-MMAE]₆ Conjugate (the Reduction Temperature in Step (1) is Different)

The method of examples 33-36 is similar to example 1, and the difference is the reduction temperature, the molar ratio of and Zn² and TCEP and/or the molar ratio of and TCEP and the antibody in step (1) which are shown as follows.

| | the reduction temperature | Zn^{2+/}TCEP | TCEP/antibody |
|---|---|---|---|
| No. | in step (1) (° C.) | (Molar ratio) | (Molar ratio) |
| E33 | 0 | 4:1 | 4:1 |
| E34 | 10 | 2:1 | 3.8:1 |
| E35 | 15 | 4:1 | 3.8:1 |
| E36 | 25 | 0.5:1 | 4:1 |

The homogeneity assays results were shown as follows:

TABLE 6
The results of homogeneity assays of examples 33-36

| | | The reduction temperature | | | | | |
|---|---|---|---|---|---|---|---|
| No. | FIG. | in step (1) (° C.) | D0(%) | D2(%) | D4(%) | D6(%) | D8(%) |
| E33 | 17A | 0 | 0 | 1.37 | 4.48 | 88.17 | 5.99 |
| E34 | 17B | 10 | 0 | 2.28 | 9.34 | 88.38 | 0 |
| E35 | 17C | 15 | 0 | 2.06 | 9.48 | 86.32 | 2.14 |
| E36 | 17D | 25 | 1.05 | 1.47 | 12.27 | 79.69 | 5.52 |

As shown in table 6, the results showed the content of D6 is up to 80%, 85%, even to 88% when the reductant temperature in step (1) is from 0° C. to 25° C.

### Examples 37-47: Preparation of Trastuzumab-[MC-VC-PAB-MMAE]₆ Conjugate by Using the Different Buffer System

The method of examples 37-47 is the same as example 1, and the difference is that the BES buffer of example 1 is replaced by different buffer of examples 37-47.

TABLE 7
the buffer system and the pH value of
the buffer system of examples 37-47

| | | The pH value of |
|---|---|---|
| Example No. | The buffer system | the buffer system |
| E37 | Bis-Tris buffer | 6.7 |
| E38 | PIPES buffer | 6.7 |
| E39 | MOPS buffer | 6.7 |
| E40 | BES buffer | 6.7 |
| E41 | HEPES buffer | 6.7 |
| E42 | DIPSO buffer | 6.7 |
| E43 | MOBS buffer | 7.4 |
| E44 | MOPSO buffer | 7.4 |
| E45 | TES buffer | 7.4 |
| E46 | ACES buffer | 7.4 |
| E47 | TAPSO buffer | 7.4 |

### Comparative Examples 5-6: Preparation of Trastuzumab-[MC-VC-PAB-MMAE]₆ Conjugate by Using the Different Buffer System

The method of comparative examples 5-6 is similar to example 1, and the difference is that the BES buffer of example 1 is replaced by different buffer of comparative examples 5-6.

TABLE 8
the buffer system and the pH value of the
buffer system of comparative examples 5-6

| Comparative | | The pH value of |
|---|---|---|
| example No. | The buffer system | the buffer system |
| C5 | PB | 6.7 |
| C6 | ADA buffer | 6.7 |

The homogeneity assays results were shown as follows:

TABLE 9
the results of homogeneity assays of examples 37-47 and comparative examples 5-6

| No. | FIG. | The buffer system | The pH | D0(%) | D2(%) | D4(%) | D6(%) | D8(%) |
|---|---|---|---|---|---|---|---|---|
| E37 | 18 | Bis-Tris buffer | 6.7 | 0 | 5 | 11 | 79 | 5 |
| E38 | 19 | PIPES buffer | 6.7 | 0 | 11 | 31 | 56 | 2 |
| E39 | 20 | MOPS buffer | 6.7 | 0 | 2 | 7 | 83 | 8 |
| E40 | 21 | BES buffer | 6.7 | 0 | 4 | 7 | 84 | 5 |
| E41 | 22 | HEPES buffer | 6.7 | 0 | 1 | 4 | 82 | 13 |
| E42 | 23 | DIPSO buffer | 6.7 | 0 | 0 | 2 | 87 | 11 |
| E43 | 24 | MOBS buffer | 7.4 | 0 | 0 | 3 | 88 | 9 |
| E44 | 25 | MOPSO buffer | 7.4 | 0 | 0 | 4 | 87 | 9 |
| E45 | 26 | TES buffer | 7.4 | 0 | 2 | 5 | 86 | 7 |
| E46 | 27 | ACES buffer | 7.4 | 0 | 0 | 5 | 87 | 8 |
| E47 | 28 | TAPSO buffer | 7.4 | 0 | 1 | 4 | 88 | 7 |
| C5 | 29 | PB | 6.7 | 2 | 36 | 46 | 16 | 0 |
| C6 | 30 | ADA buffer | 6.7 | 0 | 8 | 33 | 47 | 12 |

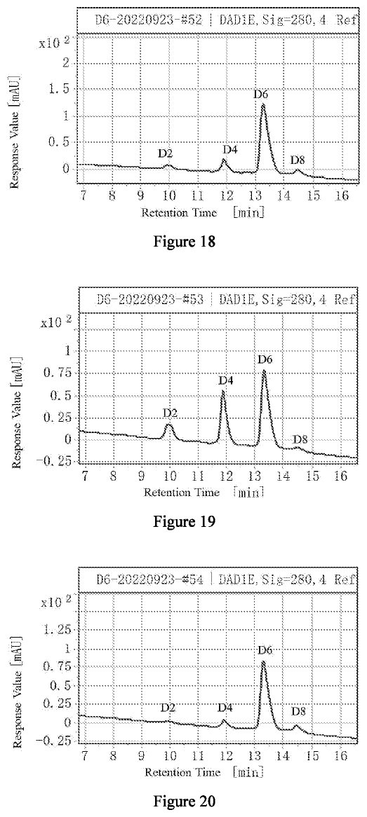

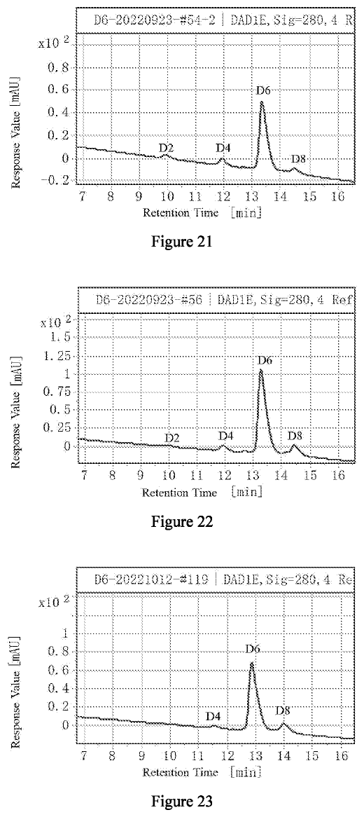

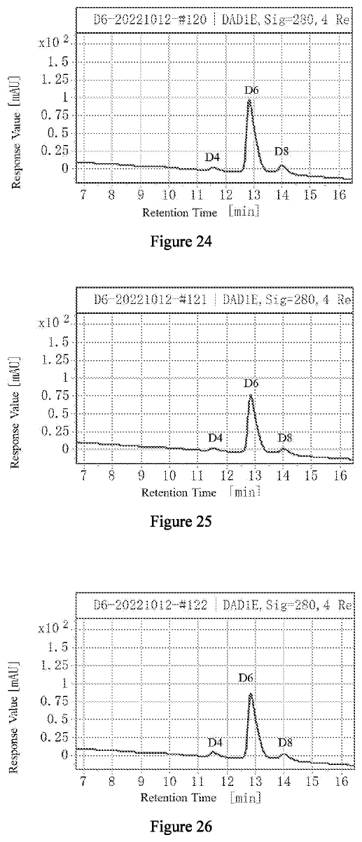

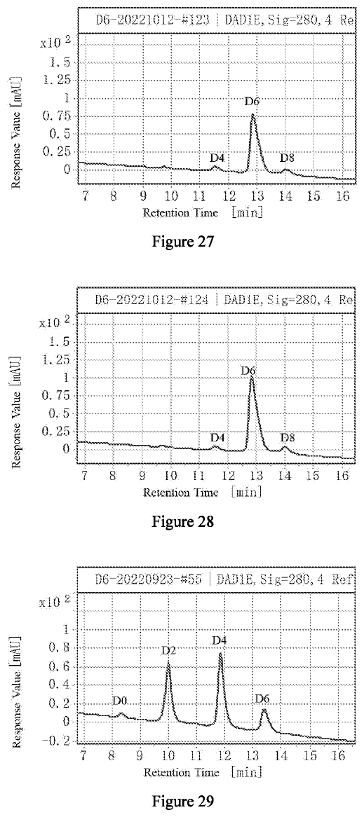

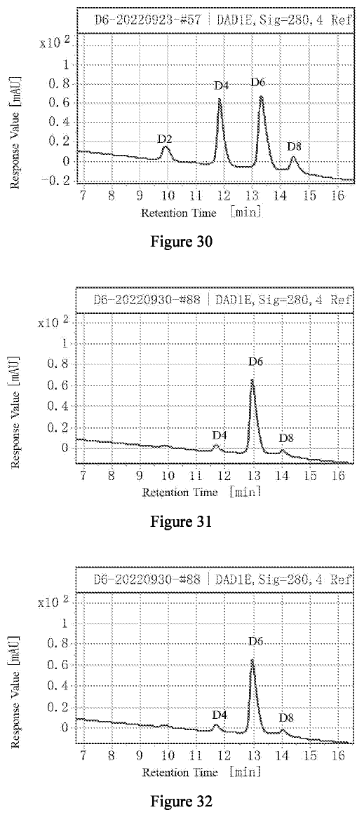

As shown in table 9, and FIGS. 18-30, the results showed the types of the buffer system will impact the content of D6 by impacting the reduction kinetics and selectivity. The buffer systems of examples 37-47 are useful to increase the content of D6.

### Examples 48-51: Preparation of Trastuzumab-[MC-VC-PAB-MMAE]₆ Conjugate at Different pH Value

The method of examples 48-51 is similar to example 1, and the difference is the pH value of the buffer system.

TABLE 10
the pH value of the buffer system of examples 48-51

| | | The pH value of |
|---|---|---|
| Example No. | The buffer system | the buffer system |
| E48 | BES buffer | 6.4 |
| E49 | BES buffer | 6.7 |
| E50 | BES buffer | 7.0 |
| E51 | BES buffer | 7.4 |

The homogeneity assays results were shown as follows:

TABLE 11
the results of homogeneity assays of examples 48-51

| Example No. | FIG. | The buffer system | The pH | D0(%) | D2(%) | D4(%) | D6(%) | D8(%) |
|---|---|---|---|---|---|---|---|---|
| E48 | 31 | BES buffer | 6.4 | 0 | 4 | 10 | 79 | 7 |
| E49 | 32 | BES buffer | 6.7 | 0 | 0 | 7 | 89 | 4 |
| E50 | 33 | BES buffer | 7.0 | 0 | 0 | 6 | 90 | 4 |
| E51 | 34 | BES buffer | 7.4 | 0 | 0 | 5 | 90 | 5 |

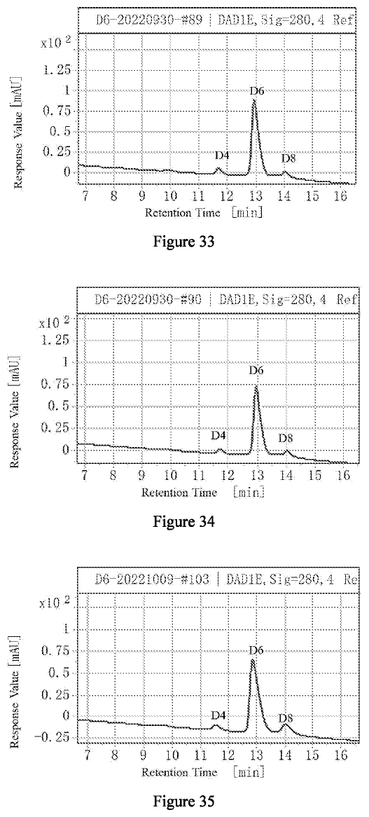

As shown in table 11, and FIGS. 31-34, the results showed the pH value of the buffer systems of examples 48-51 are useful to increase the content of D6.

### Examples 52-55: Preparation of Trastuzumab-[MC-VC-PAB-MMAE]₆ Conjugate at Different Concentration of the Buffer System

The method of examples 52-55 is similar to example 1, and the difference is the concentration of the buffer system.

TABLE 12
the concentration of the buffer system of examples 52-55

| | | The concentration |
|---|---|---|
| Example No. | The buffer system | of the buffer system |
| E52 | BES buffer | 20 mM |
| E53 | BES buffer | 40 mM |
| E54 | BES buffer | 60 mM |
| E55 | BES buffer | 80 mM |

The homogeneity assays results were shown as follows:

TABLE 13
the results of homogeneity assays of examples 52-55

| | | The buffer | | | | | | |
|---|---|---|---|---|---|---|---|---|
| Example No. | FIG. | system | concentration | D0(%) | D2(%) | D4(%) | D6(%) | D8(%) |
| E52 | 35 | BES buffer | 20 mM | 0 | 0 | 4 | 86 | 10 |
| E53 | 36 | BES buffer | 40 mM | 0 | 0 | 6 | 85 | 9 |
| E54 | 37 | BES buffer | 60 mM | 0 | 3 | 9 | 81 | 7 |
| E55 | 38 | BES buffer | 80 mM | 0 | 4 | 14 | 76 | 6 |

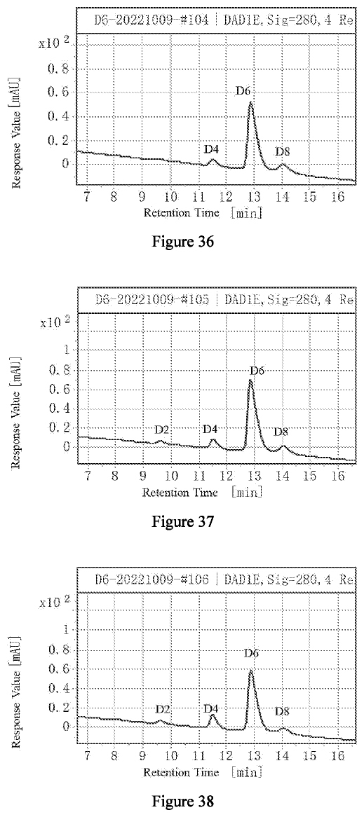

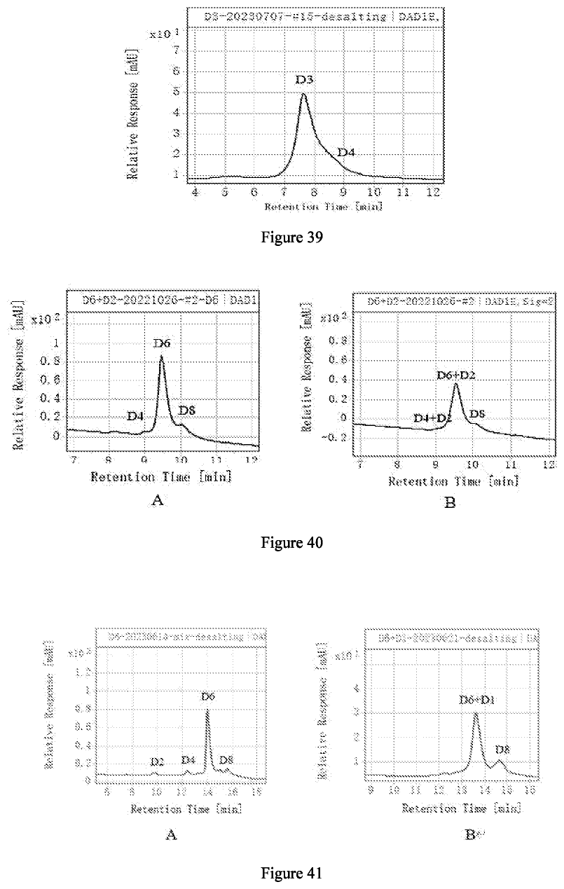

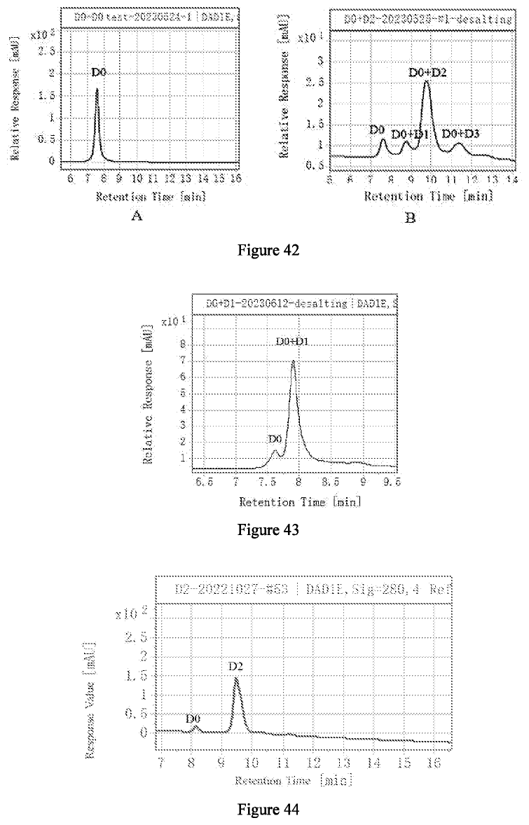

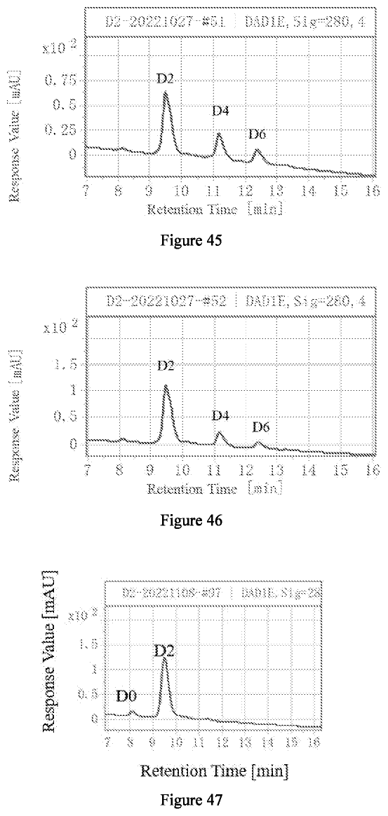

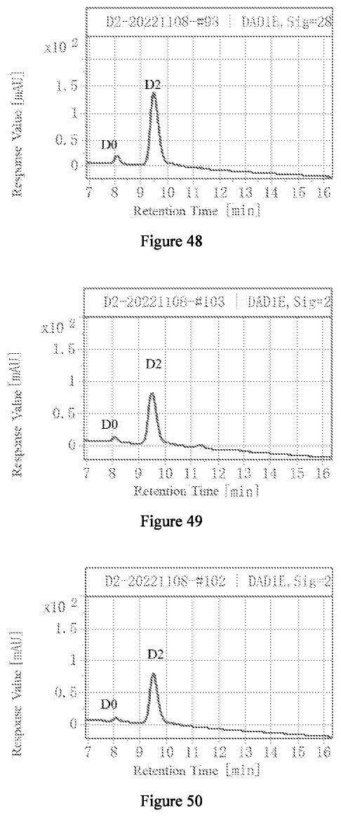

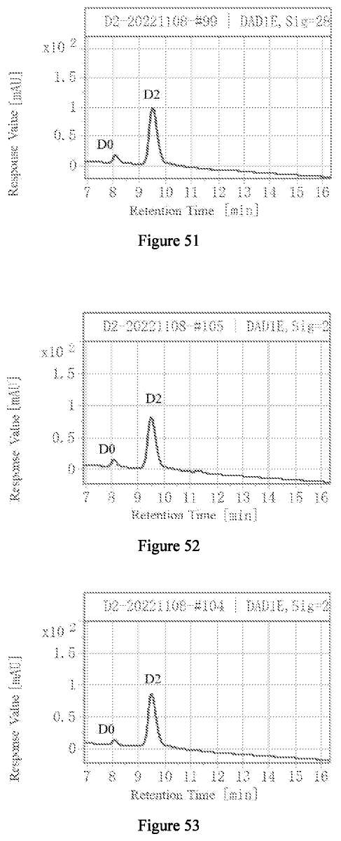

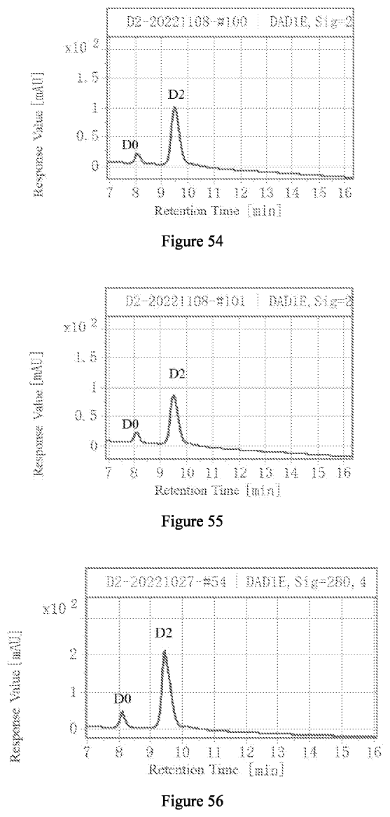

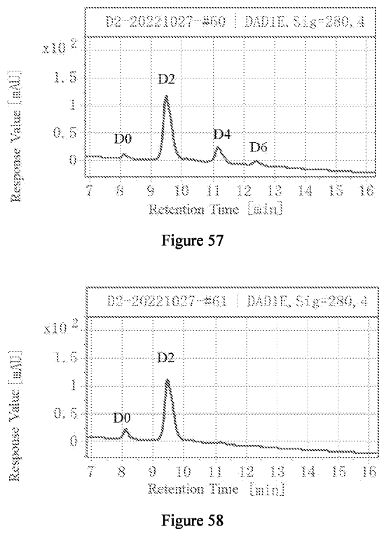

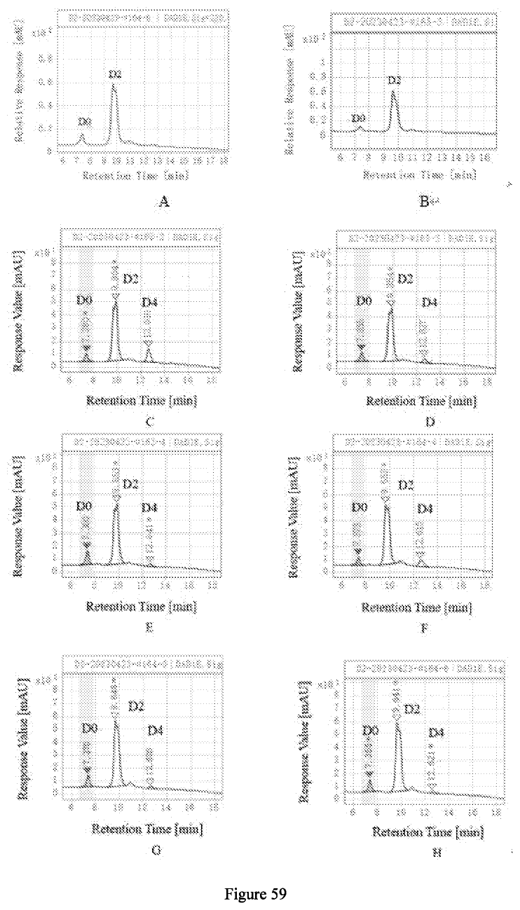

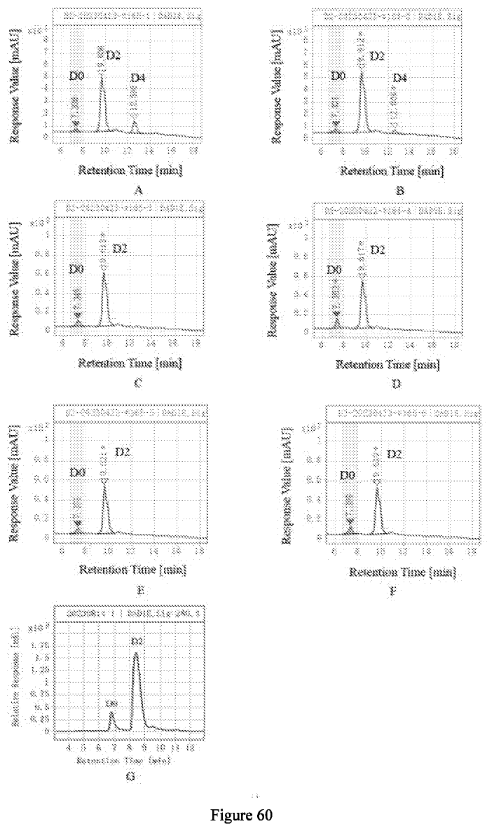

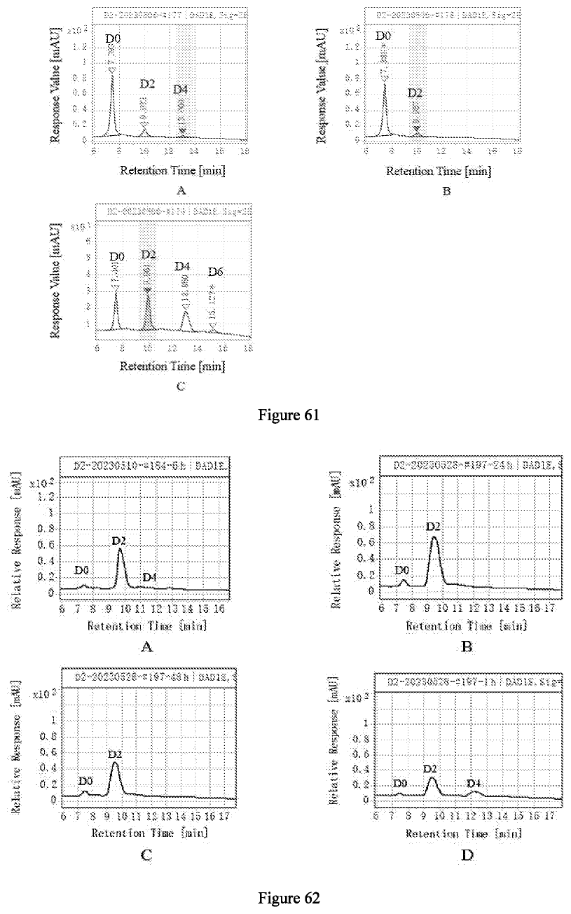

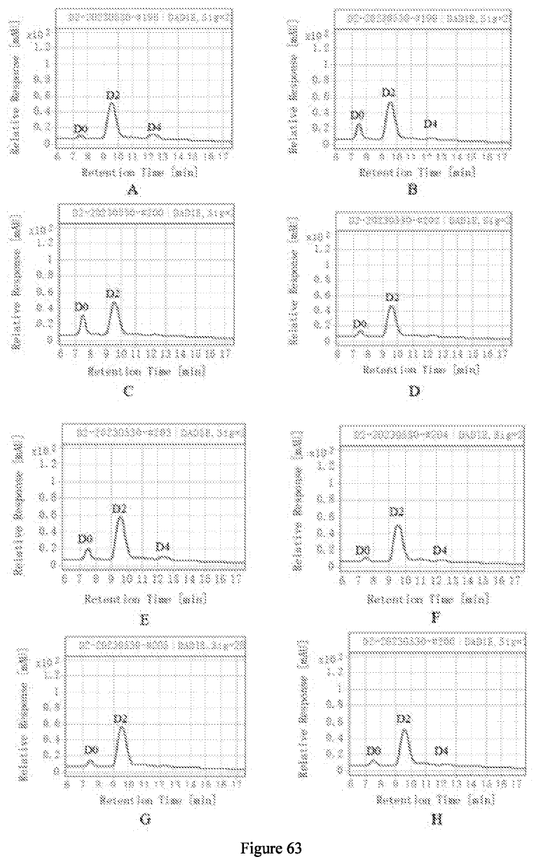

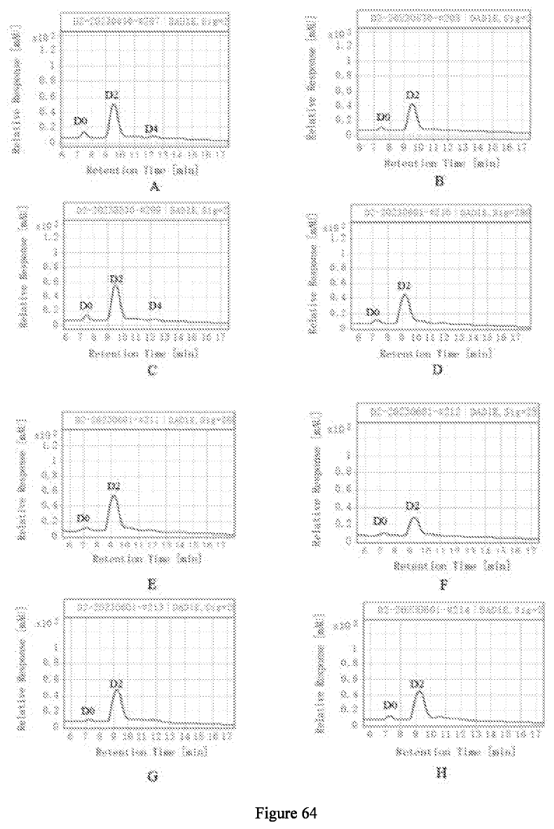

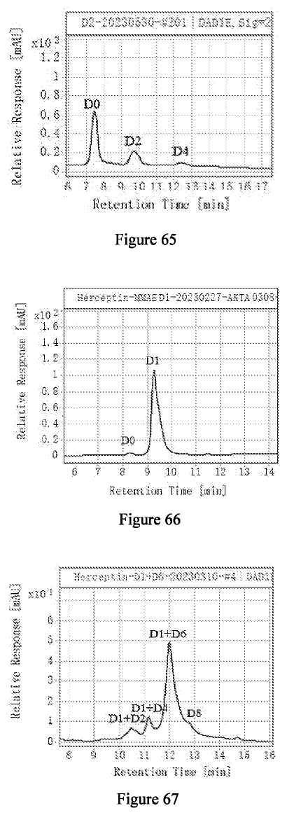

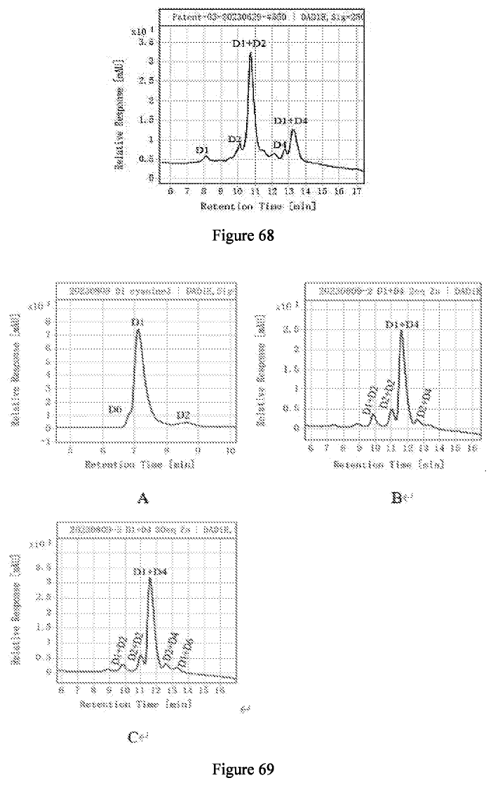

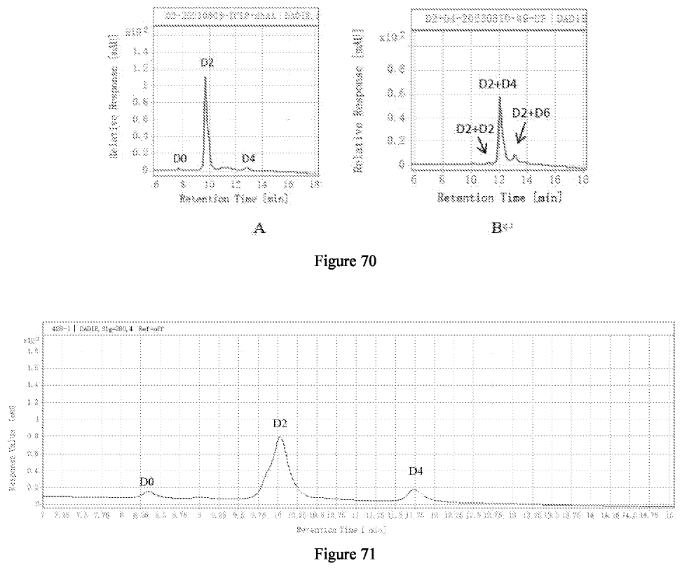

As show in table 13, and FIGS. 35-38, the results showed the concentration of the buffer systems of examples 51-54 are useful to increase the content of D6.

### Example 56: Preparation of Trastuzumab-[Bismaleimide-DBCO]₃

### 1. Synthesis of Bismaleimide-DBCO (a Thiobridge Reagent)

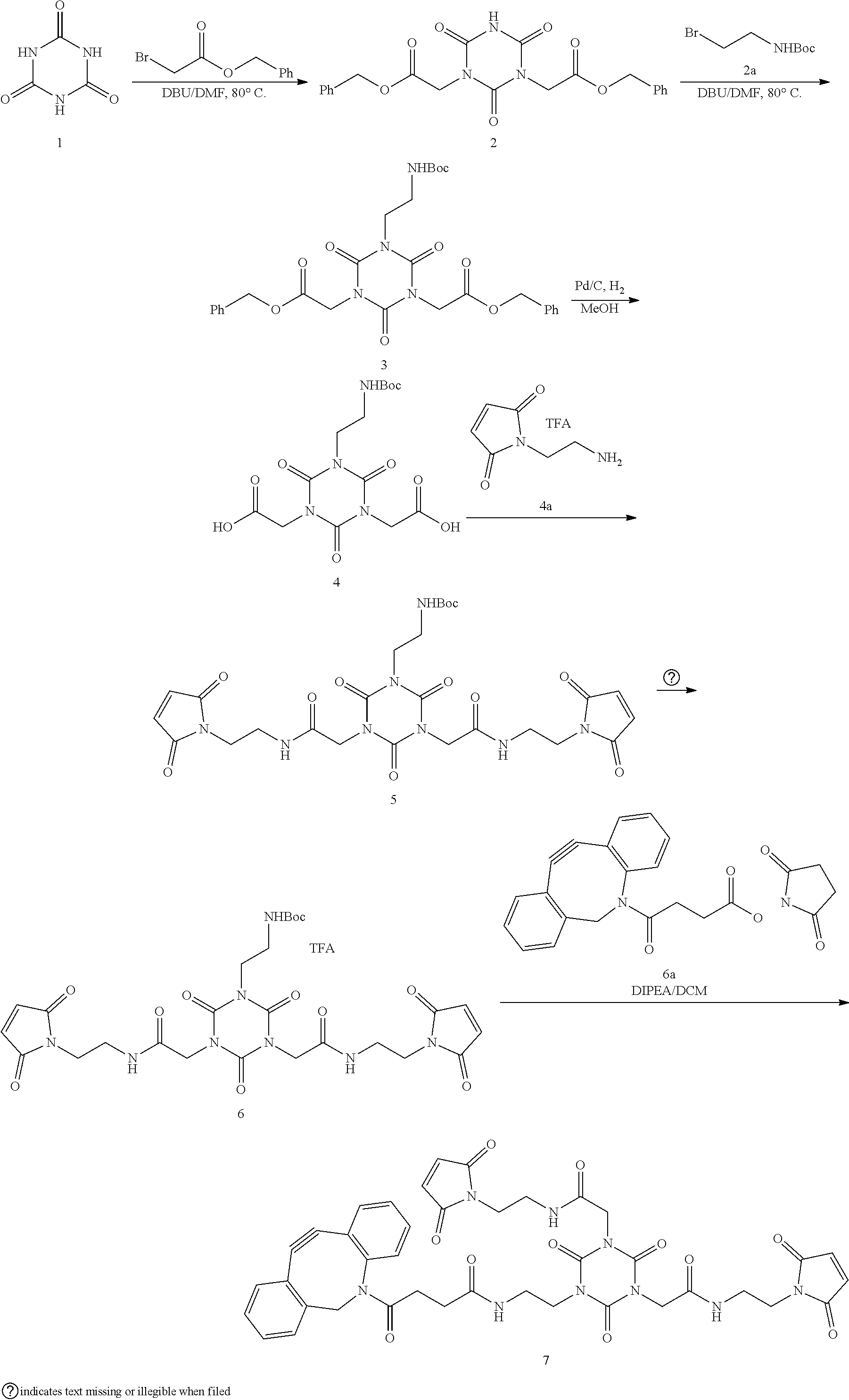

Intermediate 2: To a mixture of 1 (6.5 g, 50.0 mmol, 1.0 eq) in DMF (100 mL) was added DBU (30.7 g, 0.2 mol, 4.0 eq). The mixture was stirred for 1 h at 80° C., and Benzyl 2-bromoacetate (25.4 g, 0.11 mol, 2.2 eq). The resulting mixture was stirred for 16 h at 80° C. The mixture was poured into ice-water (600 mL), extracted with EtOAc (200 mL*3). The combined organic layer was washed with brine (200 mL), dried over Na₂SO₄ and filtered. The filtrate was concentrated, and purified by flash column (EtOAc/petroleum ether=0˜40%) to give product 2 (4.5 g, 21.0%) as white solid.

Intermediate 3: To a mixture of intermediate 2 (4.5 g, 10.58 mmol, 1.0 eq) in DMF (50 mL) was added DBU (3.22 g, 21.16 mmol, 2.0 eq). The mixture was stirred for 0.5 h at 80° C., and tert-Butyl N-(2-bromoethyl) carbamate (3.56 g, 15.87 mmol, 1.5 eq). The resulting mixture was stirred for 6 h at 80° C. TLC showed compound 1 was consumed completely. The mixture was poured into ice-water (300 mL), extracted with EtOAc (100 mL*3). The combined organic layer was washed with brine (100 mL), dried over Na₂SO₄ and filtered. The filtrate was concentrated, and purified by flash column (EtOAc/petroleum ether=0˜40%) to give product 3 (4.8 g, 79.8%) as white solid.

Intermediate 4: To a mixture of intermediate 3 (4.8 g, 8.44 mmol, 1.0 eq) in THF (50 mL) was added Pd/C (500 mg, 10% Pd/C, wetted with ca. 55% Water). The mixture was degassed 3 times and purged with H₂. The resulting mixture was stirred for 2 h at room temperature under H₂ atmosphere. TLC showed intermediate 3 was consumed completely. The mixture was filtered through a Celite pad and filtrate was concentrated to give product 4 (3.0 g, 91.5%) as white solid.

Intermediate 5: To a mixture of intermediate 4 (50 mg, 1.00 mmol, 1.0 eq) in DMF (5 mL) was added HATU (0.92 g, 2.4 mmol, 2.4 eq) and DIPEA (0.65 g, 5.0 mmol, 5.0 eq). The mixture was stirred for 0.5 h at room temperature, and 4a (0.51 g, 2.0 mmol, 2.0 eq) was added. The resulting mixture was stirred for 2 h at room temperature. LCMS showed intermediate 4 was consumed completely. The mixture was quenched by adding HCl (0.5M, 2 mL), and purified by RP-column (water/MeCN=10-70%), and the eluent was lyophilized to give product 5 (0.51 g, 80.3%) as white solid.

Intermediate 6: To a mixture of intermediate 5 (100 mg, 0.16 mmol, 1.0 eq) in DCM (2 mL) was added TFA (100 uL). The mixture was stirred for 1 hours at room temperature. LCMS showed intermediate 5 was consumed completely. The mixture was concentrated and the residue was taken up by water (10 mL), then lyophilized to give product 6 (100 mg, 97.8%) as white solid.

Compound 7 (Bismaleimide-DBCO): To a mixture of intermediate 6 (50 mg, 93.9 umol, 1.0 eq) in DCM (2 mL) was added 6a (37.8 mg, 93.9 umol, 1.0 eq) followed by DIPEA (17 uL, 93.9 umol, 1.0 eq). The mixture was stirred for 1 h at room temperature. LCMS showed intermediate 6 was consumed completely. The mixture was concentrated and the residue was purified by prep-HPLC (water/MeCN=10˜50%), and the eluent was lyophilized to give product 7 (28.8 mg, 3.410%) as white solid. MS (M+H)⁺=820.19, exact mass calc. for C₄₀H₃₇N₉O₁₁ is 819.26. ¹H NMR (400 MHz, DMSO-d6): δ 8.17 (t, J=6.0 Hz, 2H), 7.85 (d, J=6.4 Hz, 1H), 7.64 (dd, J=18.4, 7.3 Hz, 2H), 7.47 (d, J=10.3 Hz, 3H), 7.38-7.24 (m, 3H), 6.96 (s, 4H), 5.02 (d, J=14.0 Hz, 1H), 4.22 (s, 4H), 3.68 (t, J=6.6 Hz, 2H), 3.65-3.58 (m, 4H), 3.43 (t, J=6.2 Hz, 3H), 3.24-3.20 (m, 3H), 3.16-3.10 (m, 5H).

### 2. Preparation of Trastuzumab-[Bismaleimide-DBCO]₃

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and Bismaleimide-DBCO (0.045 mM) to react with reduced thiol groups resulted from step (1) at room temperature for 1 h, then recovering the product using a desalting column to afford Trastuzumab-[Bismaleimide-DBCO]₃;

The homogeneity assays results were shown as follows:

| | | The type of | | |
|---|---|---|---|---|
| NO. | FIG. | the ADCs | D3 (%) | D4 (%) |
| E56 | 39 | D3 | 86.55 | 13.45 |

As shown in the table, the result demonstrated that the content of Trastuzumab-[Bismaleimide-DBCO]₃ was generally up to 86%, which indicated the process of method was benefit for site-specific modifying the antibody with D3 and improving the homogeneity.

### Example 57: Preparation of Trastuzumab-[MC-GGFG-DXd]₆[MC-VC-PAB-MMAE]₂ (the ADC with D6+D2)

(1) ZnCl₂ (0.048 mM) and reductant TCEP (0.048 mM) were added to a solution of a monoclonal antibody Trastuzumab (0.012 mM) in BES buffer (pH7.0, 20 mM) and the reaction mixture was allowed to stay at 4 TC for 18 h;
(2) EDTA-2Na (0.6 mM) and MC-GGFG-DXd (0.1 mM) in DMA was introduced and the reaction was continued at room temperature for 1 h, then purifying the resultant product using a desalting column;
(3) The resulting product and the second reductant TCEP (0.02 mM) were incubating at room temperature for 3 h; a second linker-payload MC-VC-PAB-MMAE (0.05 mM) was added and the reaction was continued at room temperature for 2 h;
(4) purifying the resultant product using a desalting column.

Wherein about six drug molecules MC-GGFG-DXd were coupled to Trastuzumab on average, and about two drug molecules MC-VC-PAB-MMAE were coupled to Trastuzumab on average.

The homogeneity assays results were shown as follows:

| NO. | FIG. | D4 (%) | D6 (%) | D8 (%) |
|---|---|---|---|---|
| E57-step (2) | 40A | 2.24 | 88.82 | 8.93 |

| NO. | FIG. | D4 + D2 (%) | D6 + D2 (%) | D8 (%) |
|---|---|---|---|---|
| E57-step (4) | 40B | 3.58 | 87.10 | 9.32 |

As shown in the above table, the result demonstrated that the content of the ADC with D6+D2 was generally up to 87.10%, which indicated the process of method was benefit for site-specific modifying the antibody with D6+D2 and improving the homogeneity.

### Example 58: Preparation of Trastuzumab-[MC-VC-PAB-MMAE]₆[Maleimide-PEG4-N3-DBCO-Cy3]₁ (the ADC with D6+D1)

### 1. Synthesis of Dibromomaleimide-PEG4-N3

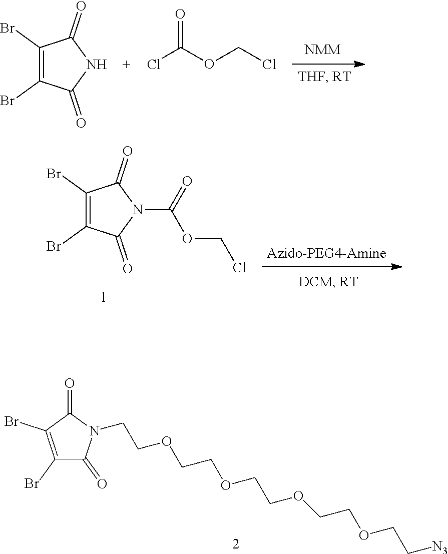 [SMILES: O=C1NC(=O)C(Br)=C1Br ; O=C(Cl)OCCl ; O=C(OCCl)N1C(=O)C(Br)=C(Br)C1=O ; [N-]=[N+]=NCCOCCOCCOCCOCCN1C(=O)C(Br)=C(Br)C1=O]

To a solution of 3,4-dibromomaleimide (127 mg, 0.5 mmol, 1 eq) and N-methylmorpholine (0.22 mL, 2 mmol, 4 eq) in THF (3.5 mL), chloromethyl chloroformate (0.18 mL, 2 mmol, 4 eq) was added and the mixture was stirred for 20 min at room temperature. Then DCM (10 mL) was added, the organic phase was washed with H₂O, dried over MgSO₄ and the solvent removed in vacuo to yield the title product 1 (139 mg, 0.4 mmol, 80%).

A solution of Azido-PEG4-Amine (105 mg, 0.4 mmol, 1 eq, Xi'an Confluore Biological Technology Co., Ltd) in dichloromethane (2 mL) was added to a stirred solution of product 1 (139 mg, 0.4 mmol, 1 eq) in dichloromethane (2 mL).

After 30 minutes, dichloromethane (6 mL) was added and the solution washed with a 0.68 M acetate buffer pH 5 (10 mL), water (1 mL), and dried with MgSO₄. Concentration in vacuo followed by purification by column chromatography (100% EtOAc as the mobile phase) yielded dibromomaleimide-PEG4-N3 (the title product 2) as a pale yellow oil (150 mg, 0.3 mmol, 75%).
2. Preparation of Trastuzumab-[MC-VC-PAB-MMAE]₆[Maleimide-PEG4-N3-DBCO-Cy3]₁
(1) ZnCl₂ (0.0408 mM) and TCEP (0.024 mM) were added toa solution of a monoclonal antibody Trastuzumab (0.012 mM) in BES buffer (pH7.0, 20 mM) and the reaction mixture was allowed to stay at 4° C. for 16 h;
(2) EDTA-2Na (0.6 mM) and MC-VC-PAB-MMAE (0.1 mM) in DMA was introduced and the reaction was continued at room temperature for 1 h, then recovering the product using a desalting column;
(3) The resulting product and reductant TCEP (0.09 mM) were incubating at room temperature for 20 h; the second thiobridge reagent with the reactive group Dibromomaleimide-PEG4-N3 (0.012 mM) was added and the reaction was continued at room temperature for 2 h; then the second linker-payload DBCO-Cy3 (0.05 mM) was added and the reaction was continued at room temperature for 4 h.
(4) purifying the resultant product using a desalting column.

Wherein about six drug molecules MC-VC-PAB-MMAE were coupled to Trastuzumab on average, and about one drug molecule Maleimide-PEG4-N3-DBCO-Cy3 was coupled to Trastuzumab on average.

The homogeneity assays results were shown as follows:

| NO. | FIG. | D2 (%) | D4 (%) | D6 (%) | D8 (%) |
|---|---|---|---|---|---|
| E58-step (2) | 41A | 4.48 | 4.97 | 86.49 | 4.06 |

| NO. | FIG. | D6 + D1 (%) | D8 (%) | / | / |
|---|---|---|---|---|---|
| E58-step (4) | 41B | 89.89 | 10.11 | / | / |

As shown in the above table, the result demonstrated that the content of the ADC with D6+D1 was generally up to 80%, 85%, even to 90%, which indicated the process of method was benefit for site-specific modifying the antibody with D6+D1 and improving the homogeneity.

### Example 59: Preparation of Trastuzumab-[Maleimide]₆[MC-VC-PAB-MMAE]₂ (The ADC with D0+D2)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and (2-Aminoethyl) maleimide (0.1 mM) to react with reduced thiol groups resulted from step (1) at room temperature for 1 h, then recovering the product using a desalting column to afford Trastuzumab-[Maleimide]₆;
(3) Trastuzumab-[Maleimide]₆ and reductant TCEP (0.02 mM) were incubating at room temperature for 18 h; a second linker-payload MC-VC-PAB-MMAE (0.05 mM) was added and the reaction was continued at room temperature for 2 h.
(4) purifying the resultant product using a desalting column. PGP-50Ti

The homogeneity assays results were shown as follows:

| NO. | FIG. | D0(%) | / | / | / |
|---|---|---|---|---|---|
| E59-step (2) | 42A | 100 | / | / | / |

| NO. | FIG. | D0 (%) | D0 + D1 (%) | D0 + D2 (%) | D0 + D3 (%) |
|---|---|---|---|---|---|
| E59-step (4) | 42B | 11.66 | 6.31 | 70.92 | 11.11 |

As shown in the above table, the result demonstrated that the content of the ADC with D0+D2 was generally up to 70.92%, which indicated the process of method was benefit for site-specific modifying the antibody with D0+D2 and improving the homogeneity.

### Example 60: Preparation of Trastuzumab-[Maleimide]₆[Maleimide-PEG4-N3-DBCO-Cy3]ᵢ (The ADC with D0+D1)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) introducing EDTA (0.6 mM) and (2-Aminoethyl) maleimide (0.1 mM) to react with reduced thiol groups resulted from step (1) at room temperature for 1 h, then recovering the product using a desalting column to afford Trastuzumab-[Maleimide]₆;
(3) Trastuzumab-[Maleimide]₂ and the second reductant TCEP (0.02 mM) were incubating at room temperature for 18 h. the second thiobridge reagent with the reactive group dibromomaleimide-PEG4-N3 (0.013 mM) to react with reduced thiol groups, the reaction temperature is 24° C. and the reaction time is 3 h;
(4) incubating resulting product and DBCO-Cy3 (0.02 mM) in MES (20 mM, pH6.7), the reaction temperature is 25° C. and the reaction time is 8 h, then recovering Trastuzumab-[Maleimide]₆[Maleimide-PEG4-N3-DBCO-Cy3] using a desalting column.

The homogeneity assays results were shown as follows:

| No. | FIG. | D0 (%) | D0 + D1 (%) |
|---|---|---|---|
| E60 | 43 | 7.04 | 92.96 |

As shown in the above table, the result demonstrated that the content of the ADC with D0+D1 was generally up to 92.96%, which indicated the process of method was benefit for site-specific modifying the antibody with D0+D1 and improving the homogeneity.

### Example 61: Preparation of Trastuzumab-[MC-VC-PAB-MMAE]₂ Conjugate by Using the Process of the Present Application

(1) TCEP (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.096 mM) to selectively re-oxidize the reduced thiol groups in Fab region resulted from step (1) at 25° C. for 1 h in darkness;
(3) Introducing EDTA (0.6 mM) and MC-VC-PAB-MMAE (0.048 mM) in DMA to react with remained thiol groups from step (2) and the reaction was incubated at room temperature for 1 h;
(4) The reaction mixture was subjected to purification using a de-salting column (Thermo, type: 40K, 0.5 mL, REF:87766, Lot SJ251704,).

The homogeneity assays results were shown as follows:

TABLE 14
the result of homogeneity assays of example 61

| Example No. | FIG. | Antibody | D0(%) | D2(%) | D4(%) | D8(%) |
|---|---|---|---|---|---|---|
| 61 | 44 | Trastuzumab | 5 | 92 | 3 | 0 |

As shown in table 12 and FIGS. 44, the results demonstrate MC-VC-PAB-MMAE is successfully linked to Trastuzumab, and that the content of D2 in example 61 is 92%. These results clearly indicate that the ADC with D2 prepared with the method of the present application has a significantly improved homogeneity.

### Examples 62-90: Preparation of Trastuzumab-[MC-VC-PAB-MMAE]₂ Conjugate by Using the Process of the Present Application

The method of examples 62-90 is similar to example 61, and the difference is the parameters in step (1) and in step (2). The different parameters are shown in the table 15. Meanwhile, the oxidation time in step (2) is 2 h in examples 76-90, and the molar ratio of the ZnCl_{z} and the antibody is 2:1 in example 90.

TABLE 15
the parameters in step (1) and (2) of examples 62-75

| | DHAA | DHAA/antibody | Zn²⁺ | Zn²⁺/antibody | The temperature, time and |
|---|---|---|---|---|---|
| No. | (mM) | (molar ratio) | (mM) | (molar ratio) | purification of oxidation |
| E62 | 0.048 | 4:1 | 0.012 | 1:1 | RT, 2 h |
| E63 | 0.054 | 4.5:1 | 0.012 | 1:1 | RT, 2 h |
| E64 | 0.072 | 6:1 | 0.012 | 1:1 | RT, 2 h |
| E65 | 0.096 | 8:1 | 0.012 | 1:1 | RT, 2 h |
| E66 | 0.096 | 8:1 | 0.012 | 1:1 | 4° C., 5 h |
| E67 | 0.096 | 8:1 | 0.012 | 1:1 | RT, 2 h, purify the oxidated |
| | | | | | products using a de-salting column |
| E68 | 0.12 | 10:1 | 0.012 | 1:1 | RT, 2 h |
| E69 | 0.12 | 10:1 | 0.012 | 1:1 | 4° C., 5 h |
| E70 | 0.12 | 10:1 | 0.012 | 1:1 | RT, 2 h, purify the oxidated |
| | | | | | products using a de-salting column |
| E71 | 0.144 | 12:1 | 0.012 | 1:1 | RT, 2 h |
| E72 | 0.168 | 14:1 | 0.012 | 1:1 | RT, 2 h |
| E73 | 0.18 | 15:1 | 0.012 | 1:1 | RT, 2 h |
| E74 | 0.096 | 8:1 | 0.024 | 2:1 | RT, 2 h |
| E75 | 0.18 | 15:1 | 0.024 | 2:1 | RT, 2 h |

Room temperature is abbreviated as “RT”

TABLE 16
the parameters in step (1) and (2) of examples 76-90

| | TCEP | TCEP/antibody | DHAA | DHAA/antibody | The reduction time |
|---|---|---|---|---|---|
| No. | (mM) | (molar ratio) | (mM) | (molar ratio) | in step (1) (h) |
| E76 | 0.072 | 6:1 | 0.192 | 16:1 | 18 |
| E77 | 0.12 | 10:1 | 0.216 | 18:1 | 18 |
| E78 | 0.048 | 4:1 | 0.084 | 7:1 | 4 |
| E79 | 0.048 | 4:1 | 0.108 | 9:1 | 4 |
| E80 | 0.048 | 4:1 | 0.132 | 11:1 | 4 |
| E81 | 0.072 | 6:1 | 0.144 | 12:1 | 4 |
| E82 | 0.072 | 6:1 | 0.168 | 14:1 | 4 |
| E83 | 0.072 | 6:1 | 0.192 | 16:1 | 4 |
| E84 | 0.12 | 10:1 | 0.168 | 14:1 | 4 |
| E85 | 0.12 | 10:1 | 0.192 | 16:1 | 4 |
| E86 | 0.12 | 10:1 | 0.216 | 18:1 | 4 |
| E87 | 0.12 | 10:1 | 0.24 | 20:1 | 4 |
| E88 | 0.12 | 10:1 | 0.264 | 22:1 | 4 |
| E89 | 0.12 | 10:1 | 0.288 | 24:1 | 4 |
| E90 | 0.06 | 5:1 | 0.144 | 12:1 | 1 |

### Comparative Examples 7-9: Preparation of Trastuzumab-[MC-VC-PAB-MMAE]₂ Conjugate (the Molar Ratio of 1 and Zn²⁺ and TCEP is Different)

The method of comparative example 7 is similar to example 80, comparative example 8 is similar to example 83, comparative example 9 is similar to example 86, the difference is that the concentration of ZnCl₂ in step (1) is 0.

TABLE 17
the results of homogeneity assays of examples 62-75

| | | | | The temperature, time | | | | | |
|---|---|---|---|---|---|---|---|---|---|
| | | DHAA/antibody | Zn²⁺/antibody | and purification of | D0 | D2 | D4 | D6 | D8 |
| No. | FIG. | (molar ratio) | (molar ratio) | oxidation | (%) | (%) | (%) | (%) | (%) |
| E62 | 45 | 4:1 | 1:1 | RT, 2 h | 1 | 64 | 22 | 13 | 0 |
| E63 | 46 | 4.5:1 | 1:1 | RT, 2 h | 2 | 76 | 15 | 7 | 0 |
| E64 | 47 | 6:1 | 1:1 | RT, 2 h | 5 | 93 | 2 | 0 | 0 |
| E65 | 48 | 8:1 | 1:1 | RT, 2 h | 6 | 94 | 0 | 0 | 0 |
| E66 | 49 | 8:1 | 1:1 | 4° C., 5 h | 6 | 87 | 5 | 2 | 0 |
| E67 | 50 | 8:1 | 1:1 | RT, 2 h, purify the | 5 | 95 | 0 | 0 | 0 |
| | | | | oxidated products using | | | | | |
| | | | | a de-salting column | | | | | |
| E68 | 51 | 10:1 | 1:1 | RT, 2 h | 9 | 91 | 0 | 0 | 0 |
| E69 | 52 | 10:1 | 1:1 | 4° C., 5 h | 8 | 90 | 2 | 0 | 0 |
| E70 | 53 | 10:1 | 1:1 | RT, 2 h, purify the | 6 | 94 | 0 | 0 | 0 |
| | | | | oxidated products using | | | | | |
| | | | | a de-salting column | | | | | |
| E71 | 54 | 12:1 | 1:1 | RT, 2 h | 10 | 90 | 0 | 0 | 0 |
| E72 | 55 | 14:1 | 1:1 | RT, 2 h | 11 | 89 | 0 | 0 | 0 |
| E73 | 56 | 15:1 | 1:1 | RT, 2 h | 11 | 89 | 0 | 0 | 0 |
| E74 | 57 | 8:1 | 2:1 | RT, 2 h | 3 | 76 | 16 | 4 | 0 |
| E75 | 58 | 15:1 | 2:1 | RT, 2 h | 8 | 89 | 3 | 0 | 0 |

TABLE 18
the results of homogeneity assays of examples 76-90 and comparative examples 7-9

| | | | | The reduction | | | | |
|---|---|---|---|---|---|---|---|---|
| | | TCEP/antibody | DHAA/antibody | time in step (1) | D0 | D2 | D4 | D6 |
| No. | FIG. | (molar ratio) | (molar ratio) | (h) | (%) | (%) | (%) | (%) |
| E76 | 59A | 6:1 | 16:1 | 18 | 10.59 | 89.41 | 0 | 0 |
| E77 | 59B | 10:1 | 18:1 | 18 | 7.33 | 92.67 | 0 | 0 |
| E78 | 59C | 4:1 | 7:1 | 4 | 6.28 | 80.22 | 13.51 | 0 |
| E79 | 59D | 4:1 | 9:1 | 4 | 9.03 | 86.18 | 4.79 | 0 |
| E80 | 59E | 4:1 | 11:1 | 4 | 11.51 | 86.42 | 2.08 | 0 |
| E81 | 59F | 6:1 | 12:1 | 4 | 6.44 | 87.12 | 6.45 | 0 |
| E82 | 59G | 6:1 | 14:1 | 4 | 9.12 | 88.57 | 2.31 | 0 |
| E83 | 59H | 6:1 | 16:1 | 4 | 10.44 | 88.16 | 1.41 | 0 |
| E84 | 60A | 10:1 | 14:1 | 4 | 3.04 | 81.78 | 15.18 | 0 |
| E85 | 60B | 10:1 | 16:1 | 4 | 4.35 | 91.25 | 4.40 | 0 |
| E86 | 60C | 10:1 | 18:1 | 4 | 7.33 | 92.67 | 0 | 0 |
| E87 | 60D | 10:1 | 20:1 | 4 | 11.79 | 88.21 | 0 | 0 |
| E88 | 60E | 10:1 | 22:1 | 4 | 8.37 | 91.63 | 0 | 0 |
| E89 | 60F | 10:1 | 24:1 | 4 | 10.08 | 89.92 | 0 | 0 |
| E90 | 60G | 5:1 | 12:1 | 1 | 11.04 | 88.96 | 0 | 0 |
| C7 | 61A | 4:1 | 11:1 | 4 | 83.25 | 13.09 | 3.66 | 0 |
| C8 | 61B | 6:1 | 16:1 | 4 | 92.13 | 7.87 | 0 | 0 |
| C9 | 61C | 10:1 | 18:1 | 4 | 29.82 | 35.56 | 30.80 | 3.82 |

As shown in examples 76-90, the content of D6 is up to 80%, even to 85%, and 90% when the molar ratio of TCEP and the antibody is from 4:1 to 10:1. Meanwhile, the reduction time in step (1) is shortened to 1 h, which is with less reduction time cost.

As shown in table 17 and table 18, the results showed the molar ratio of DHAA and antibody plays an important role in determining the content of D2 and the selective oxidation. The content of D2 is up to 60%, 70%, 80%, 85%, even to 90% or 95% when the molar ratio of DHAA and the antibody is 4:1 to 24:1.

The molar ratio of Zn²⁺ and the antibody also impacts the content of D2 and the selective oxidation. As shown in comparative examples 7-9, when the concentration of Zn²⁺ is 0, the content of D2 is as low as 7.87%. It is helpful to improve the homogeneity of the ADC with D2 that the molar ratio of Zn²⁺ and the antibody is 1:1 or 2:1.

As shown in examples 65 and 67 or examples 68 and 70, the content of the ADC of D2 increased from 89% to 95% or from 88% to 94% by purifying the oxidated products. The results demonstrated it is significant to improve the content of the ADC with D2 that purifying the oxidized products before step (3).

### Examples 91-94: Preparation of Trastuzumab-[MC-VC-PAB-MMAE]₂ Conjugate (the Oxidation Temperature and/or Time in Step (2) is Different)

The method of examples 91-94 is similar to example 61, and the difference is the oxidation temperature and/or time in step (2), which are shown in table 19.

The homogeneity assays results were shown as follows:

TABLE 19
The results of homogeneity assays of examples 91-94

| | | The oxidation temperature | | | | | |
|---|---|---|---|---|---|---|---|
| No. | FIG. | and time in step (2) | D0(%) | D2(%) | D4(%) | D6(%) | D8(%) |
| E91 | 62A | 25° C., 6 h | 5.52 | 92.28 | 2.20 | 0 | 0 |
| E92 | 62B | 4° C., 24 h | 6.81 | 93.19 | 0 | 0 | 0 |
| E93 | 62C | 4° C., 48 h | 7.77 | 92.24 | 0 | 0 | 0 |
| E94 | 62D | 37° C., 1 h | 5.28 | 73.18 | 21.54 | 0 | 0 |

As shown in table 19, the results showed the content of D6 is up to 7000, even to 90% when the oxidation temperature in step (2) is from 4° C. to 37° C., and the oxidation time in step (2) is from 1 h to 48 h.

### Examples 95-110 and Comparative Example 10: Preparation of Trastuzumab-[MC-VC-PAB-MMAE]₂ Conjugate by Using the Different Buffer System

The method of examples 95-110 and comparative example 10 is similar to example 61, and the difference is that the BES buffer of example 61 is replaced by different buffer of examples 95-110 and comparative example 10. Meanwhile, the oxidation time in step (2) is 2 h in examples 95-110 and comparative example 10.

The homogeneity assays results were shown as follows:

TABLE 20
the results of homogeneity assays of examples 95-110 and comparative example 10

| No. | FIG. | The buffer system | The pH | D0(%) | D2(%) | D4(%) | D6(%) | D8(%) |
|---|---|---|---|---|---|---|---|---|
| E95 | 63A | Bis-Tris | 6.7 | 4.68 | 83.54 | 11.78 | 0 | 0 |
| E96 | 63B | PIPES | 6.7 | 20.77 | 75.71 | 3.51 | 0 | 0 |
| E97 | 63C | PB | 6.7 | 26.00 | 74.00 | 0 | 0 | 0 |
| E98 | 63D | HEPES | 7.0 | 9.80 | 90.20 | 0 | 0 | 0 |
| E99 | 63E | MOPS | 7.0 | 12.48 | 81.43 | 6.09 | 0 | 0 |
| E100 | 63F | DIPSO | 7.4 | 5.23 | 91.09 | 3.68 | 0 | 0 |
| E101 | 63G | MOBS | 7.4 | 7.89 | 92.11 | 0 | 0 | 0 |
| E102 | 63H | MOPSO | 7.4 | 7.45 | 88.78 | 3.77 | 0 | 0 |
| E103 | 64A | TES | 7.4 | 9.20 | 86.09 | 4.71 | 0 | 0 |
| E104 | 64B | ACES | 7.4 | 6.64 | 93.36 | 0 | 0 | 0 |
| E105 | 64C | TAPSO | 7.4 | 8.16 | 88.92 | 2.93 | 0 | 0 |
| E106 | 64D | MES | 5.8 | 8.43 | 91.57 | 0 | 0 | 0 |
| E107 | 64E | MES | 6.1 | 6.47 | 93.53 | 0 | 0 | 0 |
| E108 | 64F | BES | 6.4 | 8.10 | 91.90 | 0 | 0 | 0 |
| E109 | 64G | BES | 7.4 | 4.08 | 95.92 | 0 | 0 | 0 |
| E110 | 64H | BES | 7.4 | 7.78 | 92.22 | 0 | 0 | 0 |
| C10 | 65 | ADA | 6.7 | 67.96 | 25.96 | 6.08 | 0 | 0 |

As shown in table 20, the results showed the types and the pH value of the buffer system will impact the content of D2 by impacting the reduction kinetics and selectivity. The buffer systems of examples 95-110 are useful to increase the content of D2, and the pH value of the buffer system is from 5.8 to 7.4.

### Example 111: Preparation of Trastuzumab-[Maleimide-PEG4-N3-DBCO-MMAE]₁ (the ADC with D1)

(1) TCEP (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.066 mM) to selectively re-oxidize the reduced thiol groups in Fab region resulted from step (1) at 25° C. for 2 h;
(3) Introducing EDTA (0.6 mM) and a first thiobridge reagent dibromomaleimide-PEG4-N3 (0.013 mM) to react with reduced thiol groups resulted from step (3), the reaction temperature is 25° C. and the reaction time is 1 h, then recovering the product using a desalting column to afford Trastuzumab-[Maleimide-PEG4-N3]ᵢ;
(4) incubating Trastuzumab-[Maleimide-PEG4-N3]₁ and DBCO-MMAE (0.02 mM) in BES buffer (20 mM, pH7.0), the reaction temperature is 25° C. and the reaction time is 8 h.
(5) The reaction mixture was subjected to purification using a de-salting column and AKTA with HIC chromatography.

AKTA with HIC chromatography: The ADCs purification were performed using AKTA explorer with a polar MC30-HIC butyl column (4.2 mL, 800 Å, 30 μm) (commercially available from Sepax Technologies). Solvent A was 50 mM PB and 1 M (NH4)2SO4. Solvent B was 50 mM PB and 20% v/v isopropanol. Solvent C was 50 mM PB and 2 M (NH4)2SO4. ADC sample was mixed with solvent C in 1:1 volume ratio, filtered and then loaded at a flow rate of 2 mL/min. At a flow rate of 3 mL/min at 25 TC, the target component was washed out with solvent A/solvent B between v/v 65%/35% and v/v 0%/100%, and the collected product solution was concentrated and exchanged into His buffer (20 mM, pH5.5) through ultracentrifugation.

The homogeneity assays results were shown as follows:

| No. | FIG. | D0(%) | D1(%) |
|---|---|---|---|
| E111 | 66 | 2.42 | 97.58 |

As shown in the above table, the result demonstrated that the content of the ADC with D1 was generally up to 97.58%, which indicated the process of method was benefit for site-specific modifying the antibody with D1 and improving the homogeneity.

### Example 112: Preparation of Trastuzumab-[Maleimide-PEG4-N3-DBCO-MMAE]₁[MC-GGFG-DXd]₆ (the ADC with D1+D6)

(1) introducing Trastuzumab-[Maleimide-PEG4-N3-DBCO-MMAE]₁ prepared from example 111 (0.008 mM) and TCEP (0.08 mM) in BES buffer (20 mM, pH7.0), the reaction temperature is 37° C. and the reaction time is 16 h;
(2) introducing MC-GGFG-DXd (0.14 mM) to solution from step (1), and the reaction mixture was allowed to stay at 24° C. for 1 h, then recovering Trastuzumab-[Maleimide-PEG4-N3-DBCO-MMAE]₁[MC-GGFG-DXd]₆ using a desalting column.

The homogeneity assays results were shown as follows:

| No. | FIG. | D1 + D2(%) | D1 + D4 (%) | D1 + D6 (%) | D8(%) |
|---|---|---|---|---|---|
| E112 | 67 | 5.66 | 5.98 | 82.42 | 5.94 |

As shown in the above table, the result demonstrated that the content of the ADC with D1+D6 was generally up to 82.42%, which indicated the process of method was benefit for site-specific modifying the antibody with D1+D6 and improving the homogeneity.

### Example 113: Preparation of Trastuzumab-[Maleimide-PEG4-N3-DBCO-Cy3]₁[MC-VC-PAB-MMAE]₂ (the ADC with D1+D2)

(1) TCEP (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.096 mM) to selectively re-oxidize the reduced thiol groups in Fab region resulted from step (1) at 25° C. for 2 h;
(3) Introducing EDTA (0.6 mM) and a first thiobridge reagent dibromomaleimide-PEG4-N3 (0.013 mM) to react with the reduced thiol groups resulted from step (3), the reaction temperature is 25° C. and the reaction time is 1 h, then recovering the product using a desalting column to afford Trastuzumab-[Maleimide-PEG4-N3]ᵢ;
(4) incubating Trastuzumab-[Maleimide-PEG4-N3], (0.013 mM), the reaction temperature is 25° C. and the reaction time is 1 h, and then DBCO-Cy3 (0.02 mM) in BES buffer (20 mM, pH7.0), the reaction temperature is 25° C. and the reaction time is 8 h;
(5) The reaction mixture was subjected to purification using a de-salting column;
(6) incubating ZnCl₂ (0.8 mM), the second reductant TCEP (0.011 mM) and the product from step (5) (0.008 mM) in BES buffer (pH7.0, 20 mM) and the reaction mixture was allowed to stay at 4° C. for 4 h;
(7) introducing EDTA (3 mM) to trap Zn², and introducing MC-VC-PAB-MMAE (0.048 mM) to react with the reduced thiol groups resulted from step (6), the reaction temperature is 25° C. and the reaction time is 2 h;
(8) the reaction mixture was subjected to purification using a desalting column.

The homogeneity assays results were shown as follows:

| No. | FIG. | D1 (%) | D2 (%) | D1 + D2 (%) | D4 (%) | D1 + D4 (%) |
|---|---|---|---|---|---|---|
| E113 | 68 | 2.84 | 2.39 | 71.97 | 2.49 | 20.31 |

With step (6), one of the interchain disulfide bonds in the ADC with D1 were reduced. As shown in the above table, the result demonstrated that the content of the ADC with D1+D2 was generally up to 70%, which indicated the process of method was benefit for site-specific modifying the antibody with D1+D2 and improving the homogeneity.

### Examples 114-115: Preparation of Trastuzumab-[Maleimide-PEG4-N3-DBCO-Cy3]₁[MC-VC-PAB-MMAE]₄ (the ADC with D1+D4)

(1) TCEP (0.048 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.12 mM) to selectively re-oxidize the reduced thiol groups in Fab region resulted from step (1) at 25° C. for 2 h;
(3) Introducing EDTA (0.6 mM) and a mixture of a first thiobridge reagent dibromomaleimide-PEG4-N3 (0.028 mM) and DBCO-Cy3 (0.033 mM) to react with the reduced thiol groups resulted from step (2), the reaction temperature is 25° C. and the reaction time is 2 h, then recovering the product using a desalting column to afford Trastuzumab-[Maleimide-PEG4-N3-DBCO-Cy3]ᵢ;
(4) incubating ZnCl₂ (example 113: 0.024 mM; example 114: 0.36 mM), the second reductant TCEP (0.024 mM) and the product from step (3) (0.008 mM) in BES buffer (pH7.0, 20 mM) and the reaction mixture was allowed to stay at 4° C. for 16 h;
(5) introducing EDTA (3 mM) to trap Zn²⁺, and introducing MC-VC-PAB-MMAE (0.048 mM) to react with the reduced thiol groups resulted from step (4), the reaction temperature is 25° C. and the reaction time is 2 h;
(6) the reaction mixture was subjected to purification using a desalting column.

The homogeneity assays results were shown as follows:

| No. | FIG. | D0 (%) | D1 (%) | D2 (%) | / | / |
|---|---|---|---|---|---|---|
| E114-step (3) | 69A | 5.54 | 87.75 | 6.71 | / | / |
| No. | FIG. | D1 + D2 (%) | D2 + D2 (%) | D1 + D4 (%) | D2 + D4 (%) | D1 + D6 (%) |
| E114-step (6) | 69B | 8.81 | 7.74 | 79.18 | 4.26 | / |
| E115-step (6) | 69C | 5.40 | 6.94 | 81.17 | 3.77 | 2.72 |

With step (4), two of the interchain disulfide bonds in the ADC with D1 were reduced. As shown in the above table, the result demonstrated that the content of the ADC with D1+D4 was generally up to 80%, which indicated the process of method was benefit for site-specific modifying the antibody with D1+D4 and improving the homogeneity.

### Example 116: Preparation of Trastuzumab-[MC-VC-PAB-MMAE]₂[MC-GGFG-DXd]₄ Conjugate (the ADC with D2+D4)

(1) TCEP (0.0672 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 16 h;
(2) Adding DHAA (0.12 mM) to selectively re-oxidize the reduced thiol groups in Fab region resulted from step (1) at 25° C. for 2 h;
(3) Introducing EDTA (0.6 mM) and MC-VC-PAB-MMAE (0.048 mM) in DMA to react with remained thiol groups from step (2) and the reaction was incubated at room temperature for 1 h;
(4) The reaction mixture was subjected to purification using a de-salting column; (5) incubating ZnCl₂ (0.552 mM), the second reductant TCEP (1.2 mM) and the product from step (4) in BES buffer (pH7.0, 20 mM) and the reaction mixture was allowed to stay at 4° C. for 2.5 h;
(6) introducing EDTA (3 mM) to trap Zn²⁺, and introducing MC-GGFG-DXd (0.048 mM) to react with the reduced thiol groups resulted from step (5), the reaction temperature is 25° C. and the reaction time is 2 h;
(7) the reaction mixture was subjected to purification using a desalting column.

The homogeneity assays results were shown as follows:

| No. | FIG. | D0(%) | D2 (%) | D4 (%) |
|---|---|---|---|---|
| E116-step (4) | 70A | 1.29 | 95.09 | 3.62 |

| No. | FIG. | D2 + D2(%) | D2 + D4(%) | D2 + D6 (%) |
|---|---|---|---|---|
| E116-step (7) | 70B | 1.42 | 91.27 | 7.31 |

As shown in the above table, the result demonstrated that the content of the ADC with D2(DXd)⁺D4(MMAE) was generally up to 90%, which indicated the process of method was benefit for site-specific modifying the antibody with D2+D4 and improving the homogeneity.

To sum up, the methods of the present application provide different kinds of ADCs with high homogeneity without antibody and enzymes engineering. For example, the homogeneity of the ADC with D6 is more than 55%, 65%, 70%, 80%, 85%, even to 90%, and the homogeneity of the ADC with D2 is up to 60%, 70%, 75%, even to 80%, 85%, 90% or 95%. Meanwhile, the method of the present application is compatible with current thiol-reactive linker-drug technologies with minimum conformation change and intact Fc function. Meanwhile it has simple manipulation and reduced cost.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1.-44. (canceled)

45. A method of preparing an antibody with site-specific modification, wherein the site-specific modification is that three interchain disulfide bonds within the antibody are reduced selectively, the method comprises that using tris (2-carboxyethyl) phosphine (TCEP) or salt thereof and transition metal ions together.

46. A method of preparing an antibody with site-specific modification, wherein the site-specific modification is that three interchain disulfide bonds within the antibody are reduced selectively, two of the three interchain disulfide bonds are in the Fab region and one is in the hinge region of the antibody, the method comprises that using TCEP or salt thereof and transition metal ions together.

47. A method of preparing an antibody with site-specific modification, wherein the site-specific modification is that three interchain disulfide bonds within the antibody are reduced selectively, two of the three interchain disulfide bonds are in the Fab region and one is in the hinge region of the antibody, the method comprises the following steps:
(a) incubating TCEP or salt thereof and transition metal ions in the presence of an antibody in a buffer system to selectively reduce interchain disulfide bonds within the antibody to afford the antibody bearing reduced thiol groups, the molar ratio of TCEP and the antibody is 3:1 to 15:1, optionally, the molar ratio of TCEP and the antibody is 3:1 to 6:1.

48. The method according to claim 47, further comprising introducing metal chelators after step (a).

49. The method according to claim 47, wherein, in step (a), the molar ratio of TCEP and the antibody is 3.2:1 to 5:1 or 3.5:1 to 4.4:1.

50. The method according to claim 47, wherein the incubation temperature is 0° C. to 37° C. in step (a), optionally, the incubation temperature is 0° C. to 25° C. in step (a), more optionally, the incubation temperature is 0° C. to 15° C. in step (a); and/or
the incubation time is 3 h-24 h in step (a), optionally, the incubation time is 12 h-24 h in step (a), more optionally, the incubation time is 16 h to 20 h in step (a), most optionally, the incubation time is 16 h to 18 h in step (a).

51. The method according to claim 49, wherein, in step (a), the molar ratio of TCEP and the antibody is 3:1 to 6:1, the incubation time is 10-24 h.

52. The method according to claim 47, wherein, in step (a), the molar ratio of the transition metal ions and TCEP is 0.1:1 to 30:1, optionally, the molar ratio of the transition metal ions and TCEP is 0.1:1 to 20:1, more optionally, the molar ratio of the transition metal ions and TCEP is 0.5:1 to 8:1.

53. The method according to claim 47, wherein, the buffer system is selected from a group consisting of MES buffer, Bis-Tris buffer, PIPES buffer, MOPS buffer, BES buffer, HEPES buffer, DIPSO buffer, MOBS buffer, MOPSO buffer, TES buffer, ACES buffer, TAPSO buffer, PBS, PB, Acetate buffer, BTP buffer, HEPPSO buffer, POPSO buffer, EPPS buffer or Tris buffer, optionally, the buffer system is selected from a group consisting of MES buffer, Bis-Tris buffer, MOPS buffer, BES buffer, HEPES buffer, DIPSO buffer, MOBS buffer, MOPSO buffer, TES buffer, ACES buffer or TAPSO buffer; and/or
the pH value of the buffer system is 5.5 to 8, preferably, the pH value of the buffer system is 5.8 to 7.4, more preferably, the pH value of the buffer system is 6.7 to 7.4; and/or
the concertation of the buffer system is 10 mM to 100 mM, 20 mM to 80 mM or 20 mM to 40 mM.

54. The method according to claim 47, wherein, the transition metal ions selected from a group consisting of Zn²⁺, Cd²⁺, Hg²⁺, Ni²⁺, Co²⁺ or the combination thereof, optionally, the transition metal ions are Zn²⁺.

55. The method of claim 47, further comprising the following steps:
(B1) introducing oxidant to selectively re-oxidize the reduced thiol groups resulted from step (a), optionally, re-oxidize the reduced thiol groups in Fab region of the antibody, preferably, removing the excessive oxidant to purify the oxidized products;
(C1) introducing the metal chelators and modification reagent 1 to react with the remained thiol groups resulted from step (B1), wherein, the modification reagent 1 is an end capping reagent, a first linker-payload or a first thiobridge reagent, optionally, the first thiobridge reagent bears the first linker-payload or reactive groups.

56. The method according to claim 55, further comprising the following steps:
(C2) introducing the metal chelators and a second reductant to selectively reduce the antibody from step (B1), optionally, reduce the interchain disulfide bongs in the hinge region of the antibody; or
(C2′) introducing the second reductant to reduce the interchain disulfide bonds in the product from step (C1), optionally, introducing the transition metal ions;
(D2) introducing a modification reagent 2 to react with the reduced thiol groups resulted from step (C2) or step (C2′), optionally, introducing the metal chelators, wherein, the modification reagent 2 is a second linker-payload or a second thiobridge reagent, optionally, the second thiobridge reagent bears the second linker-payload or reactive groups.

57. The method according to claim 56, wherein the first thiobridge reagent and the second thiobridge reagent independently contain at least two substituted groups allowing a re-bridging of the thiol groups.

58. The method according to claim 57, wherein the first thiobridge reagent and the second thiobridge reagent are independently selected from the group consisting of

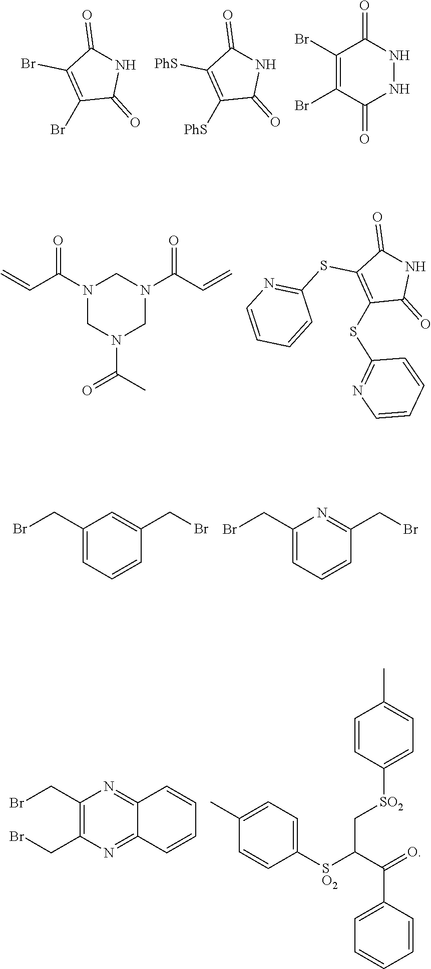

59. The method according to claim 55, wherein the reactive groups independently contain azido and/or dibenzocyclooctyne (DBCO).

60. The method according to claim 55, wherein, in step (a), the molar ratio of TCEP and the antibody is 4:1 to 15:1, the incubation time is 1 h-16 h, optionally, in step (a), the molar ratio of TCEP and the antibody is 4:1 to 10:1, the incubation time is 4 h-8 h.

61. The method according to claim 55, wherein, in step (a), the molar ratio of the transition metal ions and the antibody is 1:1 to 10:1 or 1:1 to 2:1.

62. The method according to claim 55, wherein, in step (B1), the molar ratio of the oxidant and the antibody is 2:1 to 25:1, optionally, the molar ratio of the oxidant and the antibody is 2:1 to 20:1, more optionally, the molar ratio of the oxidant and the antibody is 8:1 to 15:1.

63. The method according to claim 55, wherein, in step (B1), the oxidation temperature is 0° C. to 37° C., the oxidation time is 1 h to 48 h, optionally, the oxidation temperature is 0° C. to 30° C., the oxidation time is 1 h to 8 h.

64. The method according to claim 55, wherein, in step (B1), the oxidation reaction is in darkness.

65. The method according to claim 56, wherein, in step (C2), the molar ratio of the second reductant and the antibody is 1:1 to 2:1.

66. The method according to claim 56, wherein, in step (C2), the molar ratio of the metal chelators and the antibody is 2:1 to 120:1.

67. The method according to claim 56, wherein, in step (C2), the reduction temperature is 0° C. to 30° C., the reduction time is 1 h to 8 h.

68. The method according to claim 56, wherein, in step (C2′), the molar ratio of the second reductant and the transition metal ions is 1:0.05 to 1:40, and/or the molar ratio of the second reductant and the antibody is 2.5:1 to 20:1, and/or the incubation time is 1 h to 24 h.

69. The method according to claim 56, wherein, in step (C2′), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h.

70. The method according to claim 56, wherein, when the first thiobridge reagent bears the reactive groups, the step (C1) comprises the following steps:
introducing the metal chelators and the first thiobridge reagent bearing the reactive groups to re-bridge the reduced thiol groups resulted from step (B1), then, incubating the first linker-payload in the buffer system to react with the reactive groups of the first thiobridge group; and/or
when the second thiobridge reagent bears the reactive groups, the step (D2) comprises the following steps:
introducing the product from step (C2) or step (C2′) and the second thiobridge reagent bearing the reactive groups to re-bridge the reduced thiol groups resulted from step (C2) or step (C2′), then, incubating the second linker-payload in the buffer system to react with the reactive groups of the second thiobridge group.

71. The method according to claim 56, wherein the antibody is a monoclonal antibody, a polyclonal antibody, a mono-specific antibody or a multi-specific antibody, optionally, the antibody is a human antibody, a humanized antibody, a chimeric antibody or an antigen-binding moiety thereof, more optionally, the antibody is IgG1 or IgG4; and/or
a linker of the first linker-payload and the second linker payload is selected from any one of which the one terminal can be connected to the reduced thiol group of the antibody or the reactive groups of the thiobridge reagent, and the other terminal can be connected to the payload; and/or
the payload is selected from any one of which contains at least one substituted group allowing a connection from the payload to the linker.

72. The method of claim 47, further comprising the following step:
(b) introducing the metal chelators and the modification reagent 1 to react with the reduced thiol groups resulted from step (a).

73. The method according to claim 72, further comprising the following steps,
(c) incubating the reaction product from (b) and the second reductant in the buffer system to reduce the interchain disulfide bonds in the reaction product from (b);
(d) introducing the incubation product from step (c) and the modification reagent 2 to react with the reduced thiol groups resulted from step (c).

74. The method according to claim 72, wherein, in step (a), the molar ratio of TCEP and the antibody is 7:1 to 15:1, the incubation time is 4 h-12 h, optionally, in step (a), the molar ratio of TCEP and the antibody is 8:1 to 14:1, the incubation time is 4 h-10 h.

75. The method according to claim 73, wherein, when the first thiobridge reagent bears the reactive groups, the step (b) comprises the following steps:
introducing the metal chelators and the first thiobridge reagent bearing the reactive groups to re-bridge the reduced thiol groups resulted from step (a), then, incubating the first linker-payload in the buffer system to react with the reactive groups of the first thiobridge group; and/or
when the second thiobridge reagent bears the reactive groups, the step (d) comprises the following steps:
introducing the product from step (c) and the second thiobridge reagent bearing the reactive groups to re-bridge the reduced thiol groups resulted from step (c), then, incubating the second linker-payload in the buffer system to react with the reactive groups of the second thiobridge group.

76. The method according to claim 73, wherein the antibody is a monoclonal antibody, a polyclonal antibody, a mono-specific antibody or a multi-specific antibody, optionally, the antibody is a human antibody, a humanized antibody, a chimeric antibody or an antigen-binding moiety thereof, more optionally, the antibody is IgG1 or IgG4; and/or
a linker of the first linker-payload and the second linker payload is selected from any one of which the one terminal can be connected to the reduced thiol group of the antibody or the reactive groups of the thiobridge reagent, and the other terminal can be connected to the payload; and/or
the payload is selected from any one of which contains at least one substituted group allowing a connection from the payload to the linker.

77. An antibody with site-specific modification prepared by the method according to claim 47, wherein the antibody with site-specific modifications is the ADC with D2, the ADC with D4, the ADC with D1, the ADC with D6, the ADC with D3, the ADC with D1+D6, the ADC with D6+D2, the ADC with D6+D1, the ADC with D3+D1, the ADC with D3+D2, the ADC with D0+D6, the ADC with D0+D2, the ADC with D1+D2, the ADC with D1+D4 or the ADC with D2+D4.

78. An antibody with site-specific modification prepared by the method according to claim 55, wherein the antibody with site-specific modifications is the ADC with D2, the ADC with D4, the ADC with D1, the ADC with D1+D6, the ADC with D0+D6, the ADC with D0+D2, the ADC with D1+D2, the ADC with D1+D4 or the ADC with D2+D4.

79. An antibody with site-specific modification prepared by the method according to claim 62, wherein the antibody with site-specific modifications is the ADC with D6, the ADC with D3, the ADC with D6+D2, the ADC with D6+D1, the ADC with D3+D1, the ADC with D3+D2 or the ADC with D0+D2.

80. A pharmaceutical composition comprising the antibody with site-specific modification according to claim 77 and one or more of pharmaceutically acceptable carrier.

81. A method of preventing or treating a disease in a subject in need thereof, comprising administrating to the subject a therapeutically effective amount of the antibody with site-specific modification according to claim 77.
